(19) United States
(12) Patent Application Publication
(10) Pub. No.: US 2023/0026232 A1
(43) Pub. Date: Jan. 26, 2023

(54) AHR INHIBITORS AND USES THEREOF

(71) Applicant: Ikena Oncology, Inc., Boston, MA (US)

(72) Inventors: Alfredo C. Castro, Somerville, MA (US); James Martin NOLAN, III, Boston, MA (US); Xiaoyan Michelle ZHANG, Lexington, MA (US); Jason SAGER, Newton, MA (US)

(21) Appl. No.: 17/779,893

(22) PCT Filed: Nov. 25, 2020
(86) PCT No.: PCT/US2020/062116

§ 371 Date: May 25, 2022

Related U.S. Application Data
(60) Provisional application No. 63/106,530, filed on Oct. 28, 2020.
(60) Provisional application No. 62/940,514, filed on Nov. 26, 2019.

(51) Int. Cl. A61K 31/53; A61P 35/00; A61K 9/20

(52) U.S. Cl. CPC A61K 31/53; A61P 35/00; A61K 9/2054

(57) ABSTRACT

The present invention provides AHR inhibitors, formulations and unit dosage forms thereof, and methods of use thereof.

### CROSS-REFERENCE TO RELATED APPLICATIONS

This application claims the benefit under 35 U.S.C. § 119(e) of U.S. Provisional App. No. 62/940,514, filed on Nov. 26, 2019 and U.S. Provisional App. No. 63/106,530, filed Oct. 28, 2020, the contents of each of which are hereby incorporated by reference in their entireties.

### TECHNICAL FIELD OF THE INVENTION

The present invention relates to formulation and dosage forms of an AHR inhibitor (R)—N-(2-(5-fluoropyridin-3-yl)-8-isopropylpyrazolo[1,5-a][1,3,5]triazin-4-yl)-2,3,4,9-tetrahydro-1H-carbazol-3-amine (Compound A), and methods of use thereof.

### BACKGROUND OF THE INVENTION

Aryl hydrocarbon receptor (AHR) is a ligand-activated nuclear transcription factor that, upon binding to ligand, translocates from the cytoplasm to the nucleus and forms a heterodimer with aryl hydrocarbon receptor nuclear translocator (ARNT) (Stevens, 2009). The AHR-ARNT complex binds to genes containing dioxin response elements (DRE) to activate transcription. Numerous genes are regulated by AHR; the most well documented genes include the cytochrome P450 (CYP) genes, CYP1B1 and CYP1A1 (Murray, 2014).

Multiple endogenous and exogenous ligands are capable of binding to and activating AHR (Shinde and McGaha, 2018; Rothhammer, 2019). One endogenous ligand for AHR is kynurenine, which is generated by indoleamine 2, 3-dioxygenase 1 (IDO1) and tryptophan 2,3-dioxygenase (TDO2) from the precursor tryptophan. Many cancers over-express IDO1 and/or TDO2, leading to high levels of kynurenine. Activation of AHR by kynurenine or other ligands alters gene expression of multiple immune modulating genes leading to immunosuppression within both the innate and adaptive immune system (Opitz, 2011). Activation of AHR leads to differentiation of naïve T cells toward regulatory T cells (Tregs) over effector T cells (Funatake, 2005; Quintana 2008). It has recently been shown that activated AHR up-regulates programmed cell death protein 1 (PD-1) on CD8+ T cells to reduce their cytotoxic activity (Liu, 2018). In myeloid cells, AHR activation leads to a tolerogenic phenotype on dendritic cells (Vogel, 2013). In addition, AHR activation drives the expression of KLF4 that suppresses NF-xB in tumor macrophages and promotes CD39 expression that blocks CD8+ T cell function (Takenaka, 2019).

AHR-mediated immune suppression plays a role in cancer since its activity prevents immune cell recognition of and attack on growing tumors (Murray, 2014; Xue, 2018; Takenaka, 2019).

### SUMMARY OF THE INVENTION

It has been found that AHR inhibitor (R)—N-(2-(5-fluoropyridin-3-yl)-8-isopropylpyrazolo[1,5-a][1,3,5]triazin-4-yl)-2,3,4,9-tetrahydro-1H-carbazol-3-amine (Compound A) formulations and unit dosage forms of the invention have certain advantages in treating cancer.

Accordingly, in one aspect, the present invention provides a formulation comprising Compound A, or a pharmaceutically acceptable salt thereof. In another aspect, the present invention provides a unit dosage form comprising Compound A, or a pharmaceutically acceptable salt thereof. In another aspect, the present invention provides a method for treating cancer comprising administering a formulation or a unit dosage form as described herein.

In some embodiments, the present invention provides a method for treating cancer in a patient, comprising administering to the patient a therapeutically effective amount of Compound A, or a pharmaceutically acceptable salt thereof. In some embodiments, a method provided herein comprises administering daily to a patient about 200-1600 mg of Compound A, or a pharmaceutically acceptable salt thereof. In some embodiments, a method provided herein comprises administering once daily, or twice daily, or thrice daily, or four-times daily, Compound A, or a pharmaceutically acceptable salt thereof.

In some embodiments, the present invention provides a method for treating cancer in a patient, comprising administering to the patient a therapeutically effective amount of a metabolite of Compound A, or a pharmaceutically acceptable salt thereof, or a prodrug thereof. In some embodiments, a metabolite of Compound A is Compound B or Compound C, or pharmaceutically acceptable salts thereof.

In some embodiments, a cancer is selected from those as described herein. In some embodiments, a cancer is selected from urothelial carcinomas, including, but not limited to, bladder cancer and all transitional cell carcinomas; head and neck squamous cell carcinoma; melanoma, including, but not limited to, uveal melanoma; ovarian cancer, including, but not limited to, a serous subtype of ovarian cancer; renal cell carcinoma, including, but not limited to, clear cell renal cell carcinoma subtype; cervical cancer; gastrointestinal/stomach (GIST) cancer, including but not limited to, stomach cancer; non-small cell lung cancer (NSCLC); acute myeloid leukemia (AML); and esophageal cancers.

In some embodiments, a patient is a patient who has histologically confirmed solid tumors who has locally recurrent or metastatic disease that has progressed on or following all standard of care therapies deemed appropriate by the treating physician, or who is not a candidate for standard treatment.

In some embodiments, a patient has urothelial carcinoma and histological confirmation of urothelial carcinoma, and/or has unresectable locally recurrent or metastatic disease that has progressed on or following all standard of care therapies deemed appropriate by the treating physician (e.g., including a platinum containing regimen and checkpoint inhibitor), or who is not a candidate for standard treatment.

### BRIEF DESCRIPTION OF FIGURES

FIG. 1 depicts Thermograms for Compound A Free Base.

FIG. 2 depicts Thermograms for Compound A Hemi-Maleate Salt.

FIG. 3 depicts XRPD diffractogram of crystalline Compound A Free Base and Hemi-Maleate Salt.

FIG. 4 depicts overlaid XRPD diffractograms of crystalline Compound A Free Base, Hemi-Maleate salt and their subsequent Jet-Milled material.

FIG. 5 depicts overlaid DSC thermograms of crystalline Compound A Free Base, Hemi-Maleate salt and their subsequent Jet-Milled material.

FIG. 6 depicts PSD of Compound A Free Base and subsequent Jet-Milled Material.

FIG. 7 depicts PSD of Compound A maleate salt and subsequent Jet-Milled Material.

FIG. 8 depicts overlaid intrinsic dissolution of Compound A Free Base, Hemi-Maleate salt, and subsequent Jet-Milled material.

FIG. 9 depicts MDSC Thermograms of Compound A Free Base Feasibility SDIs.

FIG. 10 depicts MDSC Thermograms of Compound A Maleate Salt Feasibility SDIs.

FIG. 11 depicts XRPD Diffractograms of Compound A Feasibility SDIs.

FIG. 12 depicts non-sink dissolution data for Compound A Feasibility SDIs compared to bulk crystalline Compound A.

FIG. 13 depicts Tg as a Function of RH for Compound A Lead SDI Formulations.

FIG. 14 depicts t=0 Assay, Impurities Data for Compound A Lead SDI Formulations.

FIG. 15 depicts XRPD Diffractograms of Compound A SDIs after 4 Weeks Stability.

FIG. 16 depicts XRPD Diffractograms of 40:60 Compound A:HPMCAS-M SDIs after 10 Weeks Stability.

FIG. 17 depicts overlaid chromatograms of assay, impurities data for 25:70:5 Compound A:HPMCAS-L:TPGS compared to bulk crystalline API after 4 weeks stability.

FIG. 18 depicts overlaid chromatograms of assay, impurities data for 40:60 Compound A:PVP-VA compared to bulk crystalline API after 4 weeks stability.

FIG. 19 depicts overlaid chromatograms of assay, impurities data for 40:60 Compound A:HPMCAS-M compared to bulk crystalline API after 4 weeks stability.

FIG. 20 depicts Overlaid Chromatograms of Assay, Impurities Data for 25:75 Compound A:HPMCP-HP55 Compared to Bulk Crystalline API After 4 Weeks Stability.

FIG. 21 depicts Overlaid Chromatograms of Assay, Impurities Data for 40:60 Compound A:HPMCP-HP55 Compared to Bulk Crystalline API After 4 Weeks Stability.

FIG. 22 depicts Overlaid Chromatograms of Assay, Impurities Data for 40:60 Compound A:HPMCAS-M Compared to Bulk Crystalline API After 10 Weeks Stability.

FIG. 23 depicts MDSC Thermograms for Compound A Demonstration SDI.

FIG. 24 depicts XRPD Diffractograms of Compound A Demonstration SDI.

FIG. 25 depicts Compound A FB Tablet Demonstration Batch (220 mg/g Common Granulation) Process Flow Chart.

FIG. 26 depicts A. Tabletability, B. Compressibility, C. Compactibility, and D. Disintegration profiles for 50 & 150 mg Compound A: HPMCAS-M tablets made during feasibility and scale-up.

FIG. 27 depicts Non-Sink Dissolution Data for Compound A Prototype Tablets at 100 RPM.

FIG. 28 depicts Non-Sink Dissolution Data for Compound A Prototype Tablets at 150→250 RPM.

FIG. 29 depicts Compound A SDDs provide good oral exposure in Cynomolgus Macaques.

FIGS. 30A-30B demonstrate Compound A inhibits basal and kynurenine-induced activation of CYP1B1 in whole blood from human donors.

FIG. 31 depicts dose-dependent inhibition of VAG539-mediated mRNA induction by Compound A in the mouse liver and spleen.

FIG. 32 demonstrates effects of Compound A, anti-PD-1 antibody, and a combination therapy of Compound A and anti-PD-1 antibody, on B16-IDO1 Tumor Growth in C57Bl/6 mice.

FIG. 33 demonstrates effects of Compound A, anti-PD-1 antibody, and a combination therapy of Compound A and anti-PD-1 antibody, on CT26.WT Tumor Growth in BALB/cJ mice.

FIG. 34 demonstrates effects of Compound A, anti-PD-1 antibody, and a combination therapy of Compound A and anti-PD-1 antibody, on survival in the CT26.WT mouse model.

### DETAILED DESCRIPTION OF THE INVENTION

### 1. General Description of Certain Embodiments of the Invention

Compound A is a novel, synthetic, small molecule inhibitor designed to target and selectively inhibit the AHR. It has been found that there are multiple tumor types that have high levels of AHR signaling as determined by an AHR-gene signature. The high level of AHR activation caused by elevated levels of kynurenine and other ligands, as well as its role in driving an immune suppressive tumor microenvironment (TME), make AHR an attractive therapeutic target in multiple cancer types. Without wishing to be bound by any particular theory, bladder cancer can, in some embodiments, be an indication for treatment with an AHR inhibitor for multiple reasons, including 1) AHR target genes are highly differentially expressed in bladder cancer relative to normal bladder tissue, 2) it has been found that over-expression of AHR target genes is correlated with the poor overall survival in bladder cancer patients, 3) it has been found that AHR immunohistochemistry tumor microarray (TMA) analysis across 15 different tumor types revealed that bladder cancer has the highest level of AHR protein expression and AHR nuclear localization, an indicator of active AHR signaling, and 4) approximately 7% to 22% of bladder cancer patients harbor AHR gene amplification per cBioportal.

Compound A is a selective AHR antagonist being developed as an orally administered therapeutic. Compound A potently inhibits AHR activity in human and rodent cell lines (˜35-150 nM half maximal inhibitory concentration [IC50]) and is highly selective for AHR over other receptors, transporters, and kinases. In human T cell assays, Compound A induces an activated T cell state. Compound A inhibits CYP1A1 and interleukin (IL)-22 gene expression and leads to an increase in pro-inflammatory cytokines, such as IL-2 and IL-9.

The nonclinical safety of Compound A has been evaluated in a series of pharmacological, single-dose and repeated-dose toxicological studies in rodent and non-rodent species including 28-day Good Laboratory Practice (GLP) studies in rat and monkeys. Noteworthy findings in these studies of potential relevance to humans included: emesis, loose stool, dehydration, body weight loss, non-glandular stomach ulceration and edema (rats), seminiferous tubule degeneration and debris in the epididymis lumen (rats), up to 11% QTc prolongation (monkeys) and decreased thymus weights and cortical lymphocytes (monkey). All changes were resolved or resolving after 2 weeks of dosing cessation, except for the testicular changes in rats. The nonclinical safety assessment from these studies supports clinical evaluation of Compound A in humans. The initial planned dose of Compound A for this study is 200 mg once daily (QD), based on evaluation of the Compound A nonclinical safety data. Doses of 200 mg, 400 mg, 800 mg, and 1200 mg once daily (QD) have been tested in human patients with no serious adverse events (SAEs).

Accordingly, in some embodiments, the present invention provides a method for treating cancer in a patient, such as bladder cancer, comprising administering to the patient a therapeutically effective amount of Compound A, or a pharmaceutically acceptable salt thereof.

Accordingly, in some embodiments, the present invention provides a method for treating bladder cancer in a patient, comprising administering to the patient a therapeutically effective amount of Compound A, or a pharmaceutically acceptable salt thereof.

In some embodiments, the present invention provides a method for treating cancer in a patient, such as bladder cancer, comprising administering to the patient a therapeutically effective amount of a metabolite of Compound A, or a pharmaceutically acceptable salt thereof, or a prodrug thereof.

In some embodiments, the present invention provides a method for treating solid tumors in a patient, comprising administering to the patient a therapeutically effective amount of compound A, or a pharmaceutically acceptable salt thereof.

In embodiments, the present invention provides a method for treating solid tumors in a patient, comprising administering to the patient a therapeutically effective amount of a metabolite of Compound A, or a pharmaceutically acceptable salt thereof, or a prodrug thereof.

In some embodiments, the present invention provides a formulation and a unit dosage form as described herein, which comprise a Compound A, or a pharmaceutically acceptable salt thereof.

### 2. Definitions

As used herein, the term “Compound A” refers to an AHR inhibitor, (R)—N-(2-(5-fluoropyridin-3-yl)-8-isopropylpyrazolo[1,5-a][1,3,5]triazin-4-yl)-2,3,4,9-tetrahydro-1H-carbazol-3-amine, of formula:

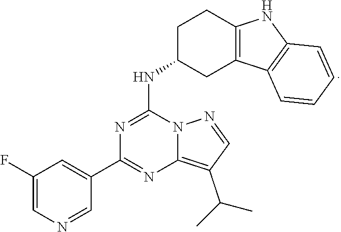 [SMILES: CC(C)c1cnn2c(N[C@@H]3CCc4[nH]c5ccccc5c4C3)nc(-c3cncc(F)c3)nc12]

In some embodiments, Compound A, or a pharmaceutically acceptable salt thereof, is amorphous. In some embodiments, Compound A, or a pharmaceutically acceptable salt thereof, is in crystal form.

As used herein, the term “a metabolite of Compound A” refers to an intermediate or end product of Compound A after metabolism. In some embodiments, a metabolite of Compound A is a compound of formula:

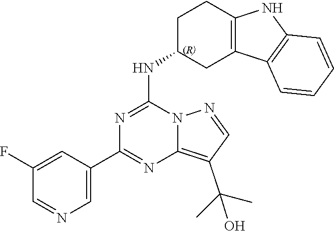

(Compound B), or a pharmaceutically acceptable salt thereof. In some embodiments, a metabolite of Compound A is a compound of formula:

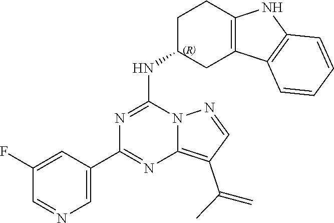

(Compound C), or a pharmaceutically acceptable salt thereof.

As used herein, the term “a prodrug thereof” refers to a compound, which produces the recited compound(s) after metabolism. In some embodiments, a prodrug of a metabolite of Compound A is a compound, which produces a metabolite of Compound A after metabolism. In some embodiments, a prodrug of a metabolite of Compound A is a compound, which produces Compound B, or a pharmaceutically acceptable salt thereof, after metabolism. In some embodiments, a prodrug of a metabolite of Compound A is a compound, which produces Compound C, or a pharmaceutically acceptable salt thereof, after metabolism.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

### 3. Description of Exemplary Methods and Uses

In some embodiments, the present invention provides a method for treating cancer in a patient, such as bladder cancer, comprising administering to the patient a therapeutically effective amount of Compound A, or a pharmaceutically acceptable salt thereof. In some embodiments, bladder cancer is urothelial carcinoma.

In some embodiments, the present invention provides a method for treating bladder cancer in a patient, comprising administering to the patient a therapeutically effective amount of Compound A, or a pharmaceutically acceptable salt thereof. In some embodiments, bladder cancer is urothelial carcinoma.

In some embodiments, the present invention provides a method for treating bladder cancer in a patient, comprising administering to the patient a therapeutically effective amount of a metabolite of Compound A, or a pharmaceutically acceptable salt thereof, or a prodrug thereof.

In some embodiments, the present invention provides a method for treating bladder cancer in a patient, comprising administering to the patient a therapeutically effective amount of Compound B, or a pharmaceutically acceptable salt thereof, or a prodrug thereof.

In some embodiments, the present invention provides a method for treating bladder cancer in a patient, comprising administering to the patient a therapeutically effective amount of Compound C, or a pharmaceutically acceptable salt thereof, or a prodrug thereof.

In some embodiments, the present invention provides a method for treating solid tumors in a patient, comprising administering to the patient a therapeutically effective amount of Compound A, or a pharmaceutically acceptable salt thereof. In some embodiments, a solid tumor is a locally advanced or metastatic solid tumor. In some embodiments, a solid tumor is a sarcoma, carcinoma, or lymphoma.

In some embodiments, the present invention provides a method for treating solid tumors in a patient, comprising administering to the patient a therapeutically effective amount of a metabolite of Compound A, or a pharmaceutically acceptable salt thereof, or a prodrug thereof.

In some embodiments, the present invention provides a method for treating solid tumors in a patient, comprising administering to the patient a therapeutically effective amount of Compound B, or a pharmaceutically acceptable salt thereof, or a prodrug thereof.

In some embodiments, the present invention provides a method for treating solid tumors in a patient, comprising administering to the patient a therapeutically effective amount of Compound C, or a pharmaceutically acceptable salt thereof, or a prodrug thereof.

In some embodiments, a solid tumor is a locally advanced or metastatic solid tumor. In some embodiments, a solid tumor is a sarcoma, carcinoma, or lymphoma.

In some embodiments, the present invention provides a method for treating cancer in a patient, comprising administering to the patient a therapeutically effective amount of Compound A, or a pharmaceutically acceptable salt thereof, or a metabolite thereof; wherein the cancer is selected from urothelial carcinoma; head and neck squamous cell carcinoma; melanoma; ovarian cancer; renal cell carcinoma; cervical cancer; gastrointestinal/stomach (GIST) cancer; non-small cell lung cancer (NSCLC); acute myeloid leukemia (AML); and esophageal cancer.

In some embodiments, the present invention provides a method for treating cancer in a patient, comprising administering to the patient a therapeutically effective amount of Compound B, or a pharmaceutically acceptable salt thereof, or a prodrug thereof, wherein the cancer is selected from urothelial carcinoma; head and neck squamous cell carcinoma; melanoma; ovarian cancer; renal cell carcinoma; cervical cancer; gastrointestinal/stomach (GIST) cancer; non-small cell lung cancer (NSCLC); acute myeloid leukemia (AML); and esophageal cancer.

In some embodiments, the present invention provides a method for treating cancer in a patient, comprising administering to the patient a therapeutically effective amount of Compound C, or a pharmaceutically acceptable salt thereof, or a prodrug thereof, wherein the cancer is selected from urothelial carcinoma; head and neck squamous cell carcinoma; melanoma; ovarian cancer; renal cell carcinoma; cervical cancer; gastrointestinal/stomach (GIST) cancer; non-small cell lung cancer (NSCLC); acute myeloid leukemia (AML); and esophageal cancer.

In some embodiments, the cancer is a urothelial carcinoma. In some embodiments, the urothelial carcinoma is bladder cancer. In some embodiments, the urothelial carcinoma is a transitional cell carcinoma.

In some embodiments, the cancer is head and neck squamous cell carcinoma.

In some embodiments, the cancer is a melanoma. In some embodiments, the melanoma is a uveal melanoma.

In some embodiments, the cancer is ovarian cancer. In some embodiments, the ovarian cancer is a serous subtype of ovarian cancer.

In some embodiments, the cancer is renal cell carcinoma. In some embodiments, the renal cell carcinoma is a clear cell renal cell carcinoma subtype.

In some embodiments, the cancer is cervical cancer.

In some embodiments, the cancer is a gastrointestinal/stomach (GIST) cancer. In some embodiments, the cancer is a stomach cancer.

In some embodiments, the cancer is non-small cell lung cancer (NSCLC). In some embodiments, the MSCLC is advanced and/or metastatic NSCLC.

In some embodiments, the cancer is esophageal cancer.

In some embodiments of a method provided herein, the method comprises administering to the patient about 200-1600 mg of Compound A, or a pharmaceutically acceptable salt thereof, daily.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, a patient or subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of nonhuman mammals), would reasonably benefit from a given treatment or therapy.

A “therapeutically effective amount” or “therapeutically effective dosage” of a drug or therapeutic agent, such as Compound A, is any amount of the drug that, when used alone or in combination with another therapeutic agent, protects a patient or subject against the onset of a disease, such as cancer, or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In preferred embodiments, a therapeutically effective amount of the drug, such as Compound A, promotes cancer regression to the point of eliminating the cancer. The term “promote(s) cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the terms “therapeutic benefit” or “benefit from therapy” refers to an improvement in one or more of overall survival, progression-free survival, partial response, complete response, and overall response rate and can also include a reduction in cancer or tumor growth or size, a decrease in severity of disease symptoms, an increase in frequency and duration of disease symptom-free periods, or a prevention of impairment or disability due to the disease affliction.

The term “patient,” as used herein, means an animal, preferably a mammal, and most preferably a human.

The term “subject,” as used herein, has the same meaning as the term “patient”.

In some embodiments, a patient is 18 years or older.

In some embodiments, a patient is a patient who has histologically confirmed solid tumors who has locally recurrent or metastatic disease that has progressed on or following all standard of care therapies deemed appropriate by the treating physician, or who is not a candidate for standard treatment.

In some embodiments, a patient has urothelial carcinoma and histological confirmation of urothelial carcinoma, and/or has unresectable locally recurrent or metastatic disease that has progressed on or following all standard of care therapies deemed appropriate by the treating physician (e.g., including a platinum containing regimen and checkpoint inhibitor), or who is not a candidate for standard treatment.

In some embodiments, a patient has urothelial carcinoma, and has histological confirmation of urothelial carcinoma, and has unresectable locally recurrent or metastatic disease that has progressed on or following all standard of care therapies deemed appropriate by the treating physician (e.g., including a platinum containing regimen and checkpoint inhibitor), or who is not a candidate for standard treatment.

In some embodiments, a patient has received a number of various prior treatment regimens. In some embodiments, a patient has measurable disease per RECIST v1.1 as assessed by the local site Investigator/radiology. In some embodiments, lesions situated in a previously irradiated area are considered measurable if progression has been demonstrated in such lesions.

In some embodiments, a patient has a tumor which can be safely accessed for multiple core biopsies. In some embodiments, a patient has not received a systemic cytotoxic chemotherapy in 2 weeks. In some embodiments, a patient has not received systemic nitrosourea or systemic mitomycin-C in 6 weeks. In some embodiments, a patient has not received a biologic therapy (e.g., antibodies) in 3 weeks.

In some embodiments, a patient has an absolute neutrophil count (ANC)≥1500/μL measured within 7 days prior to administration of a formulation and a unit dosage form as described herein. In some embodiments, a patient has Hemoglobin >8 g/dL measured within 7 days prior to administration of a formulation and a unit dosage form as described herein. In some embodiments, a patient has Platelet Count >80,000/μL measured within 7 days prior to administration of a formulation and a unit dosage form as described herein. In some embodiments, a patient has serum creatinine ≤1.5× upper limit of normal (ULN), or creatinine clearance ≥50 mL/min for patients with creatinine levels >1.5× institutional ULN (using the Cockcroft-Gault formula), measured within 7 days prior to administration of a formulation and a unit dosage form as described herein. In some embodiments, a patient has serum total bilirubin ≤1.5×ULN or direct bilirubin ≤ULN for patients with total bilirubin levels >1.5×ULN, measured within 7 days prior to administration of a formulation and a unit dosage form as described herein. In some embodiments, a patient has Aspartate aminotransferase (AST) and alanine aminotransferase (ALT)≤2.5×ULN (or ≤5×ULN if liver metastases are present), measured within 7 days prior to administration of a formulation and a unit dosage form as described herein. In some embodiments, a patient has coagulation: ≤1.5×ULN unless subject is receiving anticoagulant therapy as long as PT or aPTT is within therapeutic range of intended use of anticoagulants, measured within 7 days prior to administration of a formulation and a unit dosage form as described herein.

In some embodiments, a patient does not have clinically unstable central nervous system (CNS) tumors or brain metastasis (for the avoidance of doubt, a patient can have stable and/or asymptomatic CNS metastases). In some embodiments, a patient is not a patient who has not recovered to ≤Grade 1 or baseline from all AEs due to previous therapies. In some embodiments, a patient has ≤Grade 2 neuropathy. In some embodiments, a patient is not a patient who has an active autoimmune disease that has required systemic treatment in past 2 years with the use of disease-modifying agents, corticosteroids, or immunosuppressive drugs (for the avoidance of doubt, a patient may have used nonsteroidal anti-inflammatory drugs (NSAIDs)).

In some embodiments, a patient is not a patient who has any condition requiring continuous systemic treatment with either corticosteroids (>10 mg daily prednisone equivalents) or other immunosuppressive medications within 2 weeks prior to the present treatment (Inhaled or topical steroids and physiological replacement doses of up to 10 mg daily prednisone equivalent are permitted for a patient, in some embodiments, in the absence of active clinically significant [i.e., severe] autoimmune disease.). In some embodiments, a patient is not a patient who has any other concurrent antineoplastic treatment except for allowed local radiation of lesions for palliation (to be considered non-target lesions after treatment) and hormone ablation. In some embodiments, a patient is not a patient who has uncontrolled or life-threatening symptomatic concomitant disease (including known symptomatic human immunodeficiency virus (HIV), symptomatic active hepatitis B or C, or active tuberculosis). In some embodiments, a patient is not a patient who has undergone a major surgery within 3 weeks of the present treatment or has inadequate healing or recovery from complications of surgery prior to the present treatment. In some embodiments, a patient is not a patient who has received prior radiotherapy within 2 weeks of the present treatment. In some embodiments, a patient can be a subject who has recovered from all radiation-related toxicities, do not require corticosteroids, and have not had radiation pneumonitis. In some embodiments, a 1-week washout is permitted for palliative radiation [≤2 weeks of radiotherapy] to non-CNS disease. In some embodiments, a patient is not a patient who has received prior AHR inhibitor treatment. In some embodiments, a patient is not a patient who has potentially life-threatening second malignancy requiring systemic treatment within the last 3 years. In some embodiments, a patient is not a patient who has medical issue that limits oral ingestion or impairment of gastrointestinal function that is to significantly reduce the absorption of Compound A.

In some embodiments, a patient is not a patient who has clinically significant (i.e., active) cardiovascular disease: cerebral vascular accident/stroke (<6 months prior to the present treatment), myocardial infarction (<6 months prior to the present treatment), unstable angina, congestive heart failure (≥New York Heart Association Classification Class II), or the presence of any condition that can increase proarrhythmic risk (e.g., hypokalemia, bradycardia, heart block) including any new, unstable, or serious cardiac arrhythmia requiring medication, or other baseline arrhythmia that might interfere with interpretation of ECGs on study (e.g., bundle branch block).

In some embodiments, a patient does not have QTcF>450 msec for males and >470 msec for females on screening ECG. In some embodiments, a patient does not have a bundle branch block with QTcF>450 msec. In some embodiments, a male patient who is on stable doses of concomitant medication with known prolongation of QTcF (e.g., selective serotonin reuptake inhibitor antidepressants) does not have QTcF>470 msec.

In some embodiments, a patient does not concomitantly use a strong CYP3A inhibitor during the present treatment. In some embodiments, a strong CYP3A inhibitor is selected from the group consisting of aprepitant, clarithromycin, itraconazole, ketoconazole, nefazodone, posaconazole, telithromycin, verapamil, and voriconazole.

In some embodiments, a patient does not concomitantly use a strong CYP3A inducer during the present treatment. In some embodiments, a strong CYP3A inducer is selected from the group consisting of phenytoin, rifampin, carbamazepine, St John's Wort, bosentan, modafinil, and nafcillin.

In some embodiments, a patient does not take strong CYP3A4/5 inhibitors unless the patient can be transferred to other medications within ≥5 half-lives prior to the present treatment.

In some embodiments, a patient does not take concomitant medications that are metabolized solely through or are sensitive substrates of CYP3A4/5, CYP2C8, CYP2C9, CYP2B6, and have a narrow therapeutic window. In some embodiments, a medication, which is metabolized solely through or is a sensitive substrate of CYP3A4/5, CYP2C8, CYP2C9, CYP2B6, and has a narrow therapeutic window, is selected from the group consisting of repaglinide, warfarin, phenytoin, alfentanil, cyclosporine, diergotamine, ergotamine, fentanyl, pimozide, quinidine, sirolimus, efavirenz, bupropion, ketamine, methadone, propofol, tramadol, and tacrolimus.

In some embodiments, a patient does not take concomitant medications that are substrates of p-glycoprotein or breast cancer resistance protein (BCRP) transporters and have a narrow therapeutic window. In some embodiments, a medication, which is a substrate of p-glycoprotein or breast cancer resistance protein (BCRP) transporters and has a narrow therapeutic window, is selected from the group consisting of dabigatran, digoxin, fexofenadine(e), rosuvastatin, and sulfasalazine.

In some embodiments, a patient does not have an active infection requiring systemic therapy. In some embodiments, a patient is not a woman of child-bearing potential (WOCBP) who has a positive pregnancy test prior to the present treatment. In some embodiments, a patient is not breastfeeding or expecting to conceive or father children within the projected duration of the present treatment.

In some embodiments, a method of the present invention comprises orally administering a formulation as described herein. In some embodiments, a method of the present invention comprises administering a unit dosage form as described herein. In some embodiments, a method of the present invention comprises administering daily to a patient a formulation or a unit dosage form as described herein.

In some embodiments, a method of the present invention comprises administering daily to a patient about 100-2000 mg of compound A, or a pharmaceutically acceptable salt thereof. In some embodiments, a method of the present invention comprises administering daily to a patient about 150-1800 mg of compound A, or a pharmaceutically acceptable salt thereof. In some embodiments, a method of the present invention comprises administering daily to a patient about 200-1600 mg of compound A, or a pharmaceutically acceptable salt thereof. In some embodiments, a method of the present invention comprises administering daily to a patient about 200 mg of Compound A, or a pharmaceutically acceptable salt thereof. In some embodiments, a method of the present invention comprises administering daily to a patient about 400 mg of Compound A, or a pharmaceutically acceptable salt thereof. In some embodiments, a method of the present invention comprises administering daily to a patient about 600 mg of Compound A, or a pharmaceutically acceptable salt thereof. In some embodiments, a method of the present invention comprises administering daily to a patient about 800 mg of Compound A, or a pharmaceutically acceptable salt thereof. In some embodiments, a method of the present invention comprises administering daily to a patient about 1200 mg of Compound A, or a pharmaceutically acceptable salt thereof. In some embodiments, a method of the present invention comprises administering daily to a patient about 1600 mg of Compound A, or a pharmaceutically acceptable salt thereof. In some embodiments, a method of the present invention comprises administering a formulation or a unit dosage form as described herein once daily. In some embodiments, a method of the present invention comprises administering a formulation or a unit dosage form as described herein twice daily. In some embodiments, a method of the present invention comprises administering a formulation or a unit dosage form as described herein three times daily. In some embodiments, a method of the present invention comprises administering a formulation or a unit dosage form as described herein four times daily.

In some embodiments, where the patient is administered daily about 1200 mg of Compound A, or a pharmaceutically acceptable salt thereof, the dosing is twice daily or BID, i.e., two separate about 600 mg doses. In some embodiments, where the patient is administered daily about 1200 mg of Compound A, or a pharmaceutically acceptable salt thereof, the dosing is thrice daily or TID, i.e., three separate about 400 mg doses. In some embodiments, where the patient is administered daily about 1200 mg of Compound A, or a pharmaceutically acceptable salt thereof, the dosing is four-times daily or QID, i.e., four separate about 300 mg doses.

In some embodiments, where the patient is administered daily about 1600 mg of Compound A, or a pharmaceutically acceptable salt thereof, the dosing is twice daily or BID, i.e., two separate about 800 mg doses. In some embodiments, where the patient is administered daily about 1600 mg of Compound A, or a pharmaceutically acceptable salt thereof, the dosing is thrice daily or TID, i.e., three separate about 533 mg doses. In some embodiments, where the patient is administered daily about 1600 mg of Compound A, or a pharmaceutically acceptable salt thereof, the dosing is four-times daily or QID, i.e., four separate about 400 mg doses.

In some embodiments, a method of the present invention comprises administering a formulation or a unit dosage form as described herein, wherein there is about 4-24 hours between two consecutive administrations. In some embodiments, there is about 4, 6, 8, 12, 18, or 24 hours between two consecutive administrations.

In some embodiments, a method of the present invention comprises administering to a patient a formulation or a unit dosage form as described herein, wherein the Compound A plasma concentration is about 11,200 ng/mL or less. In some embodiments, a method of the present invention comprises administering to a patient a formulation or a unit dosage form as described herein, wherein the Compound A plasma concentration is about 9,520 ng/mL or less, about 8,400 ng/mL or less, or about 7,280 ng/mL or less. In some embodiments, a method of the present invention comprises administering to a patient a formulation or a unit dosage form as described herein, wherein the Compound A plasma concentration is about 5,600 ng/mL or less. In some embodiments, a method of the present invention comprises administering to a patient a formulation or a unit dosage form as described herein, wherein the Compound A plasma concentration is about 5,000 ng/mL or less. In some embodiments, a method of the present invention comprises administering to a patient a formulation or a unit dosage form as described herein, wherein the Compound A plasma concentration is about 4,000 ng/mL or less. In some embodiments, a method of the present invention comprises administering to a patient a formulation or a unit dosage form as described herein, wherein the Compound A plasma concentration is about 3,000 ng/mL or less. In some embodiments, a method of the present invention comprises administering to a patient a formulation or a unit dosage form as described herein, wherein the Compound A plasma concentration is about 2500 ng/mL, about 2250 ng/mL, about 2000 ng/mL, about 1750 ng/mL, about 1500 ng/mL, about 1250 ng/mL, about 1000 ng/mL, about 750 ng/mL, or about 500 ng/mL. In some embodiments, a method of the present invention comprises administering to a patient a formulation or a unit dosage form as described herein, wherein the Compound A plasma concentration is about 500 ng/mL or less.

In some embodiments, a method of the present invention comprises administering to a patient a formulation or a unit dosage form as described herein, wherein the Compound A plasma AUC is about 188,000 ng*h/mL or less. In some embodiments, a method of the present invention comprises administering to a patient a formulation or a unit dosage form as described herein, wherein the Compound A plasma AUC is about 159,800 ng*h/mL or less, about 141,000 ng*h/mL or less, or about 122,200 ng*h/mL or less. In some embodiments, a method of the present invention comprises administering to a patient a formulation or a unit dosage form as described herein, wherein the Compound A plasma AUC is about 94,000 ng*h/mL or less.

In some embodiments, a method of the present invention comprises administering daily to a patient about 100-2000 mg of a metabolite of Compound A, or a pharmaceutically acceptable salt thereof, or a prodrug thereof. In some embodiments, a method of the present invention comprises administering daily to a patient about 150-1800 mg of a metabolite of Compound A, or a pharmaceutically acceptable salt thereof, or a prodrug thereof. In some embodiments, a method of the present invention comprises administering daily to a patient about 200-1600 mg of a metabolite of Compound A, or a pharmaceutically acceptable salt thereof, or a prodrug thereof.

In some embodiments, a method of the present invention comprises administering daily to a patient about 200 mg of a metabolite of Compound A, or a pharmaceutically acceptable salt thereof, or a prodrug thereof. In some embodiments, a method of the present invention comprises administering daily to a patient about 400 mg of a metabolite of Compound A, or a pharmaceutically acceptable salt thereof, or a prodrug thereof. In some embodiments, a method of the present invention comprises administering daily to a patient about 600 mg of a metabolite of Compound A, or a pharmaceutically acceptable salt thereof, or a prodrug thereof. In some embodiments, a method of the present invention comprises administering daily to a patient about 800 mg of a metabolite of Compound A, or a pharmaceutically acceptable salt thereof, or a prodrug thereof. In some embodiments, a method of the present invention comprises administering daily to a patient about 1000 mg of a metabolite of Compound A, or a pharmaceutically acceptable salt thereof, or a prodrug thereof. In some embodiments, a method of the present invention comprises administering daily to a patient about 1200 mg of a metabolite of Compound A, or a pharmaceutically acceptable salt thereof, or a prodrug thereof. In some embodiments, a method of the present invention comprises administering daily to a patient about 1600 mg of a metabolite of Compound A, or a pharmaceutically acceptable salt thereof, or a prodrug thereof. In some embodiments, a method of the present invention comprises administering a formulation or a unit dosage form comprising a metabolite of Compound A, or a pharmaceutically acceptable salt thereof, or a prodrug thereof, once daily. In some embodiments, a method of the present invention comprises administering a formulation or a unit dosage form comprising a metabolite of Compound A, or a pharmaceutically acceptable salt thereof, or a prodrug thereof, twice daily. In some embodiments, a method of the present invention comprises administering a formulation or a unit dosage form comprising a metabolite of Compound A, or a pharmaceutically acceptable salt thereof, or a prodrug thereof, three times daily. In some embodiments, a method of the present invention comprises administering a formulation or a unit dosage form comprising a metabolite of Compound A, or a pharmaceutically acceptable salt thereof, or a prodrug thereof, four times daily.

In some embodiments, where the patient is administered daily about 1200 mg of a metabolite of Compound A, or a pharmaceutically acceptable salt thereof, or a prodrug thereof, the dosing is twice daily or BID, i.e., two separate about 600 mg doses. In some embodiments, where the patient is administered daily about 1200 mg of a metabolite of Compound A, or a pharmaceutically acceptable salt thereof, or a prodrug thereof, the dosing is thrice daily or TID, i.e., three separate about 400 mg doses. In some embodiments, where the patient is administered daily about 1200 mg of a metabolite of Compound A, or a pharmaceutically acceptable salt thereof, or a prodrug thereof, the dosing is four-times daily or QID, i.e., four separate about 300 mg doses.

In some embodiments, where the patient is administered daily about 1600 mg of a metabolite of Compound A, or a pharmaceutically acceptable salt thereof, or a prodrug thereof, the dosing is twice daily or BID, i.e., two separate about 800 mg doses. In some embodiments, where the patient is administered daily about 1600 mg of a metabolite of Compound A, or a pharmaceutically acceptable salt thereof, or a prodrug thereof, the dosing is thrice daily or TID, i.e., three separate about 533 mg doses. In some embodiments, where the patient is administered daily about 1600 mg of a metabolite of Compound A, or a pharmaceutically acceptable salt thereof, or a prodrug thereof, the dosing is four-times daily or QID, i.e., four separate about 400 mg doses.

In some embodiments, a method of the present invention comprises administering a formulation or a unit dosage form comprising a metabolite of Compound A, or a pharmaceutically acceptable salt thereof, or a prodrug thereof, wherein there is about 4-24 hours between two consecutive administrations. In some embodiments, there is about 4, about 6, about 8, about 12, about 18, or about 24 hours between two consecutive administrations of a formulation or a unit dosage form comprising a metabolite of Compound A, or a pharmaceutically acceptable salt thereof, or a prodrug thereof.

In some embodiments, the present invention provides a use of Compound A, or a pharmaceutically acceptable salt thereof, or a formulation or unit dosage form thereof, for the treatment of solid tumors and/or cancers, such as bladder cancer. In some embodiments, a formulation or unit dosage form of Compound A, or a pharmaceutically acceptable salt thereof, is as described herein. In some embodiments, the present invention provides a use of a metabolite of Compound A, or a pharmaceutically acceptable salt thereof, or a prodrug thereof, for the treatment of solid tumors and/or cancers, such as bladder cancer. In some embodiments, the present invention provides a use of a metabolite of Compound A, or a pharmaceutically acceptable salt thereof, or a prodrug thereof, in the manufacture of a formulation or a unit dosage form as described herein for the treatment of cancer. In some embodiments, a patient having a solid tumor and/or a cancer, such as bladder cancer, is as described herein.

### 4. Description of Exemplary Formulations and Dosage Forms

In some embodiments, the present invention provides a formulation and/or unit dosage form comprising Compound A, or a pharmaceutically acceptable salt thereof. In some embodiments, a Compound A formulation of the invention is a spray dried intermediate (SDI) formulation comprising Compound A, or a pharmaceutically acceptable salt thereof. In some embodiments, a Compound A unit dosage form of the invention is a tablet comprising Compound A, or a pharmaceutically acceptable salt thereof. In some embodiments, a tablet of the present invention is an immediate release (IR) tablet.

In some embodiments, a tablet of the present invention comprises Compound A free base. In some embodiments, an SDI formulation of the present invention comprises Compound A free base. In some embodiments, Compound A free base is amorphous. In some embodiments, Compound A free base is in crystal form.

In some embodiments, a tablet of the present invention comprises a pharmaceutically acceptable salt of Compound A. In some embodiments, an SDI formulation of the present invention comprises a pharmaceutically acceptable salt of Compound A. In some embodiments, a pharmaceutically acceptable salt of Compound A is amorphous. In some embodiments, a pharmaceutically acceptable salt of Compound A is in crystal form.

In some embodiments, a tablet of the present invention comprises Compound A hemi-maleate salt. In some embodiments, an SDI formulation of the present invention comprises Compound A hemi-maleate salt. In some embodiments, Compound A hemi-maleate salt is amorphous. In some embodiments, Compound A hemi-maleate salt is in crystal form.

In some embodiments, a tablet of the present invention comprises an amorphous solid dispersion of Compound A, or a pharmaceutically acceptable salt thereof, manufactured by spray drying. In some embodiments, a dispersion-containing tablet of the present invention provided enhanced oral bioavailability of Compound A.

In some embodiments, a tablet of the invention comprises a pharmaceutically acceptable polymer. In some embodiments, an SDI formulation of the invention comprises a pharmaceutically acceptable polymer. In some embodiment, a pharmaceutically acceptable polymer is polyvinylpyrrolidone/vinyl acetate copolymer (PVP-VA). In some embodiment, a pharmaceutically acceptable polymer is hypromellose (HPMC). In some embodiment, a pharmaceutically acceptable polymer is hypromellose phthalate (HPMCP-55). In some embodiment, a pharmaceutically acceptable polymer is hypromellose acetate succinate MG grade (HPMCAS-M). In some embodiment, a pharmaceutically acceptable polymer is hypromellose acetate succinate LG grade (HPMCAS-L). In some embodiment, a pharmaceutically acceptable polymer is vitamin E TPGS (TPGS). In some embodiment, a pharmaceutically acceptable polymer is microcrystalline Cellulose (MCC).

In some embodiments, an SDI formulation comprises about 5, 10, 15, 20, 25, 30, 35, 40, 45, 50, 55, 60, 65, 70, 75, 80, 85, 90, or 95% wt Compound A, or a pharmaceutically acceptable salt thereof. In some embodiments, an SDI formulation comprises about 10-75% wt Compound A, or a pharmaceutically acceptable salt thereof. In some embodiments, an SDI formulation comprises about 10-70, 15-65, 15-60, 20-55, 20-50, 25-45, or 25-40% wt Compound A, or a pharmaceutically acceptable salt thereof.

In some embodiments, an SDI formulation comprises a pharmaceutically acceptable polymer at about 5, 10, 15, 20, 25, 30, 35, 40, 45, 50, 55, 60, 65, 70, 75, 80, 85, 90, or 95% wt. In some embodiments, an SDI formulation comprises a pharmaceutically acceptable polymer at about 5-95, 10-90, 15-85, 20-85, 25-85, 30-80, 35-80, 40-80, 45-75, 50-75, 55-75, or 60-75% wt. In some embodiments, a pharmaceutically acceptable polymer in an SDI formulation is selected from PVP-VA, HPMC, HPMCP-55, HPMCAS-M, TPGS, and HPMCAS-L. In some embodiments, an SDI formulation comprises a pharmaceutically acceptable polymer selected from PVP-VA, HPMC, HPMCP-55, HPMCAS-M, and HPMCAS-L at about 60-75% wt. In some embodiments, an SDI formulation comprises TPGS at about 5 wt %.

In some embodiments, the present invention provides an SDI formulation comprising about 40:60 (wt %) Compound A or a pharmaceutically acceptable salt thereof: HPMCAS-L. In some embodiments, the present invention provides an SDI formulation comprising about 25:75 (wt %) Compound A or a pharmaceutically acceptable salt thereof: HPMCAS-L.

In some embodiments, the present invention provides an SDI formulation comprising about 40:60 (wt %) Compound A or a pharmaceutically acceptable salt thereof: HPMCAS-M. In some embodiments, the present invention provides an SDI formulation comprising about 25:75 (wt %) Compound A or a pharmaceutically acceptable salt thereof: HPMCAS-M. In some embodiments, the present invention provides an SDI formulation comprising about 40:60 (wt %) Compound A free base: HPMCAS-M.

In some embodiments, the present invention provides an SDI formulation comprising about 40:60 (wt %) Compound A or a pharmaceutically acceptable salt thereof: PVP-VA. In some embodiments, the present invention provides an SDI formulation comprising about 25:75 (wt %) Compound A or a pharmaceutically acceptable salt thereof: PVP-VA.

In some embodiments, the present invention provides an SDI formulation comprising about 40:60 (wt %) Compound A or a pharmaceutically acceptable salt thereof: HPMCP. In some embodiments, the present invention provides an SDI formulation comprising about 25:75 (wt %) Compound A or a pharmaceutically acceptable salt thereof: HPMCP.

In some embodiments, the present invention provides an SDI formulation comprising about 40:60 (wt %) Compound A or a pharmaceutically acceptable salt thereof: HPMC. In some embodiments, the present invention provides an SDI formulation comprising about 25:75 (wt %) Compound A or a pharmaceutically acceptable salt thereof: HPMC.

In some embodiments, the present invention provides an SDI formulation comprising about 25:70:5 (wt %) Compound A or a pharmaceutically acceptable salt thereof: HPMCAS-L Vit E TPGS.

In some embodiments, the present invention provides an SDI formulation comprising about 25:70:5 (wt %) Compound A or a pharmaceutically acceptable salt thereof: HPMCAS-M:Vit E TPGS.

In some embodiments, the present invention provides an SDI formulation comprising about 25:70:5 (wt %) Compound A or a pharmaceutically acceptable salt thereof: PVP-VA:Vit E TPGS.

In some embodiments, an SDI formulation of the present invention is selected from those described in Example 1 below. In some embodiments, an SDI formulation of the present invention is selected from those listed Tables 20-22, 26, 29, and 45. In some embodiments, an SDI formulation of the present invention provides Cₘₐₓ FaSSIF, C₂₁₀, and/or AUC₃₅₋₂₁₀ FaSSIF in non-sink dissolution at about the ranges as described in Table 20. In some embodiments, an SDI formulation of the present invention provides Tg at an elevated humidity condition at about the ranges as described in Table 21. In some embodiments, an SDI formulation of the present invention provides an impurity profile as described in Table 22. In some embodiments, an SDI formulation of the present invention is 40:60 wt % Compound A:HPMCAS-M with an impurity profile selected from those described in Tables 26 and 29. In some embodiments, an SDI formulation of the present invention is selected from those listed in Table 45 at the ranges of the concentrations and AUCs as described in Table 45.

In some embodiments, the present invention provides a Compound A free base or hemi-maleate with a particle size distribution (PSD) at about the Dx values as described in table 15. In some embodiments, a Compound A free base has a PSD of about 8.27 um Dx(10). In some embodiments, a Compound A free base has a PSD of about 88.0 um Dx(50). In some embodiments, a Compound A free base has a PSD of about 245 um Dx(90). In some embodiments, a Compound A free base has a PSD of about 0.83 um Dx(10). In some embodiments, a Compound A free base has a PSD of about 3.3 um Dx(50). In some embodiments, a Compound A free base has a PSD of about 13.0 um Dx(90). In some embodiments, a Compound A hemi-maleate salt has a PSD of about 3.25 um Dx(10). In some embodiments, a Compound A hemi-maleate salt has a PSD of about 18.4 um Dx(50). In some embodiments, a Compound A hemi-maleate salt has a PSD of about 213.0 um Dx(90). In some embodiments, a Compound A hemi-maleate salt has a PSD of about 0.62 um Dx(10). In some embodiments, a Compound A hemi-maleate salt has a PSD of about 1.8 um Dx(50). In some embodiments, a Compound A hemi-maleate salt has a PSD of about 9.0 um Dx(90).

In some embodiments, the present invention provides a 40:60 w/w % Compound A:HMPCAS-M spray dried dispersion (SDD). In some embodiments, a 40:60 w/w % Compound A:HMPCAS-M SDD has a PSD of about 3.9 um Dv10 measured by laser diffraction. In some embodiments, a 40:60 w/w % Compound A:HMPCAS-M SDD has a PSD of about 12.9 um Dv50 measured by laser diffraction. In some embodiments, a 40:60 w/w % Compound A:HMPCAS-M SDD has a PSD of about 43.3 um Dv90 measured by laser diffraction. In some embodiments, a 40:60 w/w % Compound A:HMPCAS-M SDD has a PSD of about 20.7 um D[4,3] measured by laser diffraction. In some embodiments, a 40:60 w/w % Compound A:HMPCAS-M SDD has an average Tg of about 99.3° C. measured by thermal analysis (MDSC).

In some embodiments, a tablet of the invention comprises an SDI formulation of the invention, and a pharmaceutically acceptable excipient or carrier. In some embodiments, a tablet of the invention comprises about 25-85 wt % of an SDI formulation of the invention. In some embodiments, a tablet of the invention comprises about 25, 30, 35, 40, 45, 50, 55, 60, 65, 70, 75, 80, or 85 wt % of an SDI formulation of the invention. In some embodiments, a tablet of the invention comprises about 30-80, 35-75, 40-70, 45-70, 50-65, or 55-65 wt % of an SDI formulation of the invention.

In some embodiments, a tablet of the invention comprises MCC at about 5-30 wt %. In some embodiments, a tablet of the invention comprises MCC at about 5, 10, 15, 20, 25, or 30 wt %. In some embodiments, a tablet of the invention comprises MCC at about 10-25 or 10-20 wt %. In some embodiments, a tablet of the invention comprises MCC at about 11.5, 15.5, 16.5, 19.5, or 20.5 wt %.

In some embodiments, a tablet of the invention comprises a filler. In some embodiments, a filler is selected from mannitol and lactose, or a hydrate thereof. In some embodiments, a filler is lactose monohydrate. In some embodiments, a tablet comprises a filler at about 10-25 wt %. In some embodiments, a tablet comprises a filler at about 10, 15, 20, or 25 wt %. In some embodiments, a tablet comprises a filler at about 15-20 wt %. In some embodiments, a tablet comprises a filler at about 15.5, 16.5, 19.5, or 20.5 wt %.

In some embodiments, a tablet of the invention comprises a disintegrant. In some embodiments, a disintegrant is croscarmellose sodium (Ac-Di-Sol). In some embodiments, a tablet comprises a disintegrant at about 0.5-10 wt %. In some embodiments, a tablet comprises a disintegrant at about 0.5, 2, 4, 6, 8, or 10 wt %. In some embodiments, a tablet comprises a disintegrant at about 0.5-4 wt %. In some embodiments, a tablet comprises a disintegrant at about 1, 2, or 4 wt %.

In some embodiments, a tablet of the invention comprises a thickening agent. In some embodiments, a thickening agent is Cab-O-Sil. In some embodiments, a tablet comprises a thickening agent at about 0.5-5 wt %. In some embodiments, a tablet comprises a thickening agent at about 0.5, 1, 1.5, 2, 3, 4, or 5 wt %. In some embodiments, a tablet comprises a thickening agent at about 0.5-1.5 wt %. In some embodiments, a tablet comprises a thickening agent at about 2 wt %.

In some embodiments, a tablet of the invention comprises sodium stearyl fumarate. In some embodiments, a tablet comprises sodium stearyl fumarate at about 0.5-5 wt %. In some embodiments, a tablet comprises sodium stearyl fumarate at about 0.5, 1, 1.5, 2, 3, 4, or 5 wt %. In some embodiments, a tablet comprises sodium stearyl fumarate at about 0.5-1.5 wt %. In some embodiments, a tablet comprises sodium stearyl fumarate at about 1 wt %.

In some embodiments, a tablet of the invention comprises a binder. In some embodiments, a binder is HPC Nisso SSL SFP. In some embodiments, a tablet comprises a binder at about 0.5-8 wt %. In some embodiments, a tablet comprises a binder at about 0.5, 1, 1.5, 2, 3, 4, 5, 6, 7, or 8 wt %. In some embodiments, a tablet comprises a binder at about 3-5 wt %. In some embodiments, a tablet comprises a binder at about 4 wt %.

In some embodiments, the present invention provides an IR tablet which has a full release in about 10 minutes in a sink dissolution test. An example of sink dissolution test is described herein. In some embodiments, an IR tablet of the present invention has a full release in about 9, 8, 7, 6, or 5 minutes in a sink dissolution test. In some embodiments, an IR tablet of the present invention has a full release in about 4 minutes in a sink dissolution test. In some embodiments, an IR tablet of the present invention has a full release in about 3 minutes in a sink dissolution test. In some embodiments, an IR tablet of the present invention has a full release in about 2 minutes in a sink dissolution test. In some embodiments, an IR tablet of the present invention has a full release in about 1 minute in a sink dissolution test.

In some embodiments, a tablet of the present invention comprises one or more pharmaceutically acceptable excipient or carrier, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide. In some embodiments, an IR tablet of the present invention comprises one or more pharmaceutically acceptable excipient or carrier including, but are not limited to, starches, sugars, micro-crystalline cellulose, diluents, granulating agents, lubricants, binders, and disintegrating agents. It will be understood by those in the art that some substances serve more than one purpose in a pharmaceutical composition. For instance, some substances are binders that help hold a tablet together after compression, yet are also disintegrants that help break the tablet apart once it reaches the target delivery site. Selection of excipients and amounts to use may be readily determined by the formulation scientist based upon experience and consideration of standard procedures and reference works available in the art.

In certain embodiments, a tablet of the present invention is manufactured using standard, art-recognized tablet processing procedures and equipment. In certain embodiments, the method for forming the tablets is direct compression of a powdered, crystalline and/or granular composition comprising a solid form provided herein, alone or in combination with one or more excipients or carriers, such as, for example, carriers, additives, polymers, or the like. In certain embodiments, as an alternative to direct compression, the tablets may be prepared using wet granulation or dry granulation processes. In certain embodiments, the tablets are molded rather than compressed, starting with a moist or otherwise tractable material. In certain embodiments, compression and granulation techniques are used. In some embodiments, a tablet of the present invention is manufactured using the process described in Example 1 below.

Suitable binders include, but are not limited to, starch (including potato starch, corn starch, and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone (PVP), cellulosic polymers (including hydroxypropyl cellulose (HPC), hydroxypropylmethylcellulose (HPMC), methyl cellulose, ethyl cellulose, hydroxyethyl cellulose (HEC), carboxymethyl cellulose and the like), veegum, carbomer (e.g., carbopol), sodium, dextrin, guar gum, hydrogenated vegetable oil, magnesium aluminum silicate, maltodextrin, polymethacrylates, povidone (e.g., KOLLIDON, PLASDONE), microcrystalline cellulose, among others. Binding agents also include, e.g., acacia, agar, alginic acid, cabomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein.

Suitable forms of microcrystalline cellulose include, but are not limited to, the materials sold as AVICEL-PH-101, AVICEL-PH-103 AVICEL RC-581, AVICEL-PH-105 (FMC Corporation, Marcus Hook, Pa.), and mixtures thereof. In some embodiment, a specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Suitable fillers include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In certain embodiments, a tablet of the present invention comprises one or more diluents. Suitable diluents include dicalcium phosphate, calcium sulfate, lactose, cellulose, kaolin, mannitol, sodium chloride, dry starch, microcrystalline cellulose (e.g., AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol and talc, among others. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-.beta.-cyclodextrin, talc, tragacanth, trehalose, and xylitol.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof.

In some embodiments, a tablet of the present invention comprises one or more lubricants. Suitable lubricants include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof.

In some embodiments, a tablet of the present invention comprises one or more glidants. Suitable glidants include, but are not limited to, colloidal silicon dioxide (CAB-O-SIL), and asbestos-free talc.

In some embodiments, a tablet of the present invention comprises one or more coloring agents. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

In some embodiments, a tablet of the present invention comprises one or more flavoring agents. Suitable flavoring agents include, but are not limited to, natural flavors extracted from plants, such as fruits, and synthetic blends of compounds which produce a pleasant taste sensation, such as peppermint and methyl salicylate.

In certain embodiments, a tablet of the present invention comprises one or more sweetening agents. Suitable sweetening agents include, but are not limited to, sucrose, lactose, mannitol, syrups, glycerin, and artificial sweeteners, such as saccharin and aspartame.

In certain embodiments, a tablet of the present invention comprises one or more emulsifying agents. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate.

In certain embodiments, a tablet of the present invention comprises one or more suspending and dispersing agents. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone.

In certain embodiments, a tablet of the present invention comprises one or more preservatives. Suitable preservatives include, but are not limited to, glycerin, methyl and propylparaben, benzoic add, sodium benzoate and alcohol.

In certain embodiments, a tablet of the present invention comprises one or more wetting agents. Suitable wetting agents include, but are not limited to, propylene glycol monostearate, sorbitan monooleate, diethylene glycol monolaurate, and polyoxyethylene lauryl ether.

In certain embodiments, a tablet of the present invention comprises one or more solvents. Suitable solvents include, but are not limited to, glycerin, sorbitol, ethyl alcohol, and syrup.

In certain embodiments, a tablet of the present invention comprises one or more non-aqueous liquids. Suitable non-aqueous liquids utilized in emulsions include, but are not limited to, mineral oil and cottonseed oil.

In certain embodiments, a tablet of the present invention comprises one or more organic acids. Suitable organic acids include, but are not limited to, citric and tartaric acid.

In certain embodiments, a tablet of the present invention comprises one or more sources of carbon dioxide. Suitable sources of carbon dioxide include, but are not limited to, sodium bicarbonate and sodium carbonate.

In certain embodiments, a tablet of the present invention can be a multiple compressed tablet, an enteric-coating tablet, or a sugar-coated or film-coated tablet. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

A tablet of the present invention can be prepared from the active ingredient in powdered, crystalline, or granular forms, alone or in combination with one or more carriers or excipients described herein, including binders, disintegrants, controlled-release polymers, lubricants, diluents, and/or colorants.

Components of a tablet of the present invention can be intragranular or extragranular. In some embodiments, a tablet comprises intragranularly Compound A, HPMCAS-M, microcrystalline cellulose, lactose monohydrate, colloidal silicon dioxide, croscarmellose sodium, and sodium stearyl fumarate. In some embodiments, a tablet comprises extragranularly Microcrystalline cellulose and Sodium Stearyl Fumarate. In some embodiments, the present invention provides a tablet with the following formula:

| Component | | w/w % |
|---|---|---|
| Intragranular | 40:60 Compound A:HPMCAS-M SDI | 55.00 |
| Total: 95.50 | Microcrystalline cellulose (Avicel PH-105) | 16.50 |
| | Lactose monohydrate (Flow Lac 90) | 20.50 |
| | Colloidal Silicon Dioxide (Cab-O-Sil) | 2.00 |
| | Croscarmellose Sodium (Ac-Di-Sol) | 1.00 |
| | Sodium Stearyl Fumarate | 0.50 |
| Extragranular | Microcrystalline cellulose (Avicel PH-200) | 4.00 |
| Total: 4.50 | Sodium Stearyl Fumarate | 0.50 |

| | |
|---|---|
| Tablet Total | 100.00 |

In some embodiments, a tablet of the present invention comprises about 10-250 mg of Compound A. In some embodiments, a tablet of the present invention comprises about 10, 20, 30, 40, 50, 60, 70, 80, 90, 100, 110, 120, 130, 140, 150, 160, 170, 180, 190, 200, 210, 220, 230, 240, or 250 mg of Compound A. In some embodiments, a tablet of the present invention comprises about 25-200 mg of compound A. In some embodiments, a tablet of the present invention comprises about 50-150 mg of Compound A.

In some embodiments, a tablet of the present invention is selected from those described in Example 1 below. In some embodiments, a tablet of the present invention is selected from those listed Tables 35, 36, and 40.

### 5. Methods and Uses for Treating Cancer

In some embodiments, the present invention provides a method for treating cancer in a patient comprising orally administering to the patient a formulation as described herein. In some embodiments, the present invention provides a method for treating cancer in a patient comprising orally administering to the patient a unit dosage form as described herein. In some embodiments, the present invention provides a method for treating cancer in a patient comprising orally administering to the patient a tablet as described herein.

The cancer or proliferative disorder or tumor to be treated using the methods and uses described herein include, but are not limited to, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

A cancer to be treated using the methods and uses described herein can be selected from urothelial carcinomas, including, but not limited to, bladder cancer and all transitional cell carcinomas; head and neck squamous cell carcinoma; melanoma, including, but not limited to, uveal melanoma; ovarian cancer, including, but not limited to, a serous subtype of ovarian cancer; renal cell carcinoma, including, but not limited to, clear cell renal cell carcinoma subtype; cervical cancer; gastrointestinal/stomach (GIST) cancer, including but not limited to, stomach cancer; non-small cell lung cancer (NSCLC); acute myeloid leukemia (AML); and esophageal cancers.

In some embodiments, a cancer is a urothelial carcinoma. In some embodiments, a cancer is bladder cancer. In some embodiments, a cancer is a transitional cell carcinoma. In some embodiments, a cancer is head and neck squamous cell carcinoma. In some embodiments, a cancer is a melanoma. In some embodiments, a cancer is a uveal melanoma. In some embodiments, a cancer is ovarian cancer. In some embodiments, a cancer is a serous subtype of ovarian cancer. In some embodiments, a cancer is renal cell carcinoma. In some embodiments, a cancer is a clear cell renal cell carcinoma subtype. In some embodiments, a cancer is cervical cancer. In some embodiments, a cancer is a gastrointestinal/stomach (GIST) cancer. In some embodiments, a cancer is a stomach cancer. In some embodiments, a cancer is non-small cell lung cancer (NSCLC). In some embodiments, a cancer is advanced and/or metastatic NSCLC. In some embodiments, a cancer is an esophageal cancer.

Cancer includes, in some embodiments, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

In certain embodiments, the cancer is selected from bladder cancer, breast cancer (including TNBC), cervical cancer, colorectal cancer, chronic lymphocytic leukemia (CLL), diffuse large B-cell lymphoma (DLBCL), esophageal adenocarcinoma, glioblastoma, head and neck cancer, leukemia (acute and chronic), low-grade glioma, lung cancer (including adenocarcinoma, non-small cell lung cancer, and squamous cell carcinoma), Hodgkin's lymphoma, non-Hodgkin lymphoma (NHL), melanoma, multiple myeloma (MM), ovarian cancer, pancreatic cancer, prostate cancer, renal cancer (including renal clear cell carcinoma and kidney papillary cell carcinoma), and stomach cancer.

In some embodiments, the cancer is small cell lung cancer, non-small cell lung cancer, colorectal cancer, multiple myeloma, acute myeloid leukemia (AML), acute lymphoblastic leukemia (ALL), pancreatic cancer, liver cancer, hepatocellular cancer, neuroblastoma, other solid tumors or other hematological cancers.

In some embodiments, the cancer is small cell lung cancer, non-small cell lung cancer, colorectal cancer, multiple myeloma, or AML.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See on the worldwide web at clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See on the worldwide web at clinicaltrials.gov/ct2/show/study/NCT02488759; see also on the worldwide web at clinicaltrials.gov/ct2/show/study/NCT0240886; on the worldwide web at clinicaltrials.gov/ct2/show/NCT02426892)

In some embodiments, the methods or uses described herein inhibit or reduce or arrest the growth or spread of a cancer or tumor. In some embodiments, the tumor or cancer is treated by arresting, reducing, or inhibiting further growth of the tumor. In some embodiments, the cancer or tumor is treated using the methods or uses described herein by reducing the size (e.g., volume or mass) of the cancer or tumor by at least 5%, at least 10%, at least 25%, at least 50%, at least 75%, at least 90%, at least 95%, at least 96%, at least 97%, at least 98%, or at least 99% relative to the size of the cancer or tumor prior to treatment. In some embodiments, cancers or tumors are treated using the methods or uses described herein by reducing the quantity of the cancers or tumors in the patient by at least 5%, at least 10%, at least 25%, at least 50%, at least 75%, at least 90%, at least 95%, at least 96%, at least 97%, at least 98%, or at least 99% relative to the quantity of tumors prior to treatment.

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

In some embodiments, a patient treated using the methods or uses described herein exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after administration of Compound A, or a pharmaceutically acceptable salt thereof. In some embodiments, a patient treated using the methods or uses described herein exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about 14 months, at least about 16 months, at least about 18 months, at least about 20 months, at least about 22 months, at least about two years, at least about three years, at least about four years, or at least about five years after administration of Compound A, or a pharmaceutically acceptable salt thereof. In some embodiments, a patient treated using the methods or uses described herein exhibits an objective response rate (ORR) of at least about 15%, at least about 20%, at least about 25%, at least about 30%, about 35%, about 40%, about 45%, about 50%, about 55%, about 60%, about 65%, about 70%, about 75%, about 80%, about 85%, about 90%, about 95%, or about 100%.

In some embodiments, a patient treated using the methods or uses described herein exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after administration of a metabolite of Compound A, or a pharmaceutically acceptable salt thereof, or a prodrug thereof. In some embodiments, a patient treated using the methods or uses described herein exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about 14 months, at least about 16 months, at least about 18 months, at least about 20 months, at least about 22 months, at least about two years, at least about three years, at least about four years, or at least about five years after administration of a metabolite of Compound A, or a pharmaceutically acceptable salt thereof, or a prodrug thereof. In some embodiments, a patient treated using the methods or uses described herein exhibits an objective response rate (ORR) of at least about 15%, at least about 20%, at least about 25%, at least about 30%, about 35%, about 40%, about 45%, about 50%, about 55%, about 60%, about 65%, about 70%, about 75%, about 80%, about 85%, about 90%, about 95%, or about 100%.

The following examples are provided for illustrative purposes only and are not to be construed as limiting this invention in any manner.

### EXEMPLARY EMBODIMENTS OF THE INVENTION

Embodiment 1. A spray dried intermediate (SDI) formulation comprising compound A,

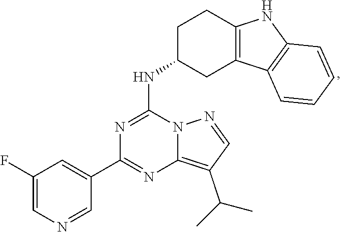 [SMILES: CC(C)c1cnn2c(N[C@@H]3CCc4[nH]c5ccccc5c4C3)nc(-c3cncc(F)c3)nc12]

or a pharmaceutically acceptable salt thereof, and a pharmaceutically acceptable polymer.
Embodiment 2. The SDI formulation of Embodiment 1, comprising compound A free base.
Embodiment 3. The SDI formulation of Embodiment 1, comprising compound A hemi-maleate.
Embodiment 4. The SDI formulation of any one of Embodiments 1-3, wherein the pharmaceutically acceptable polymer is selected from PVP-VA, HPMC, HPMCP-55, HPMCAS-M, TPGS, HPMCAS-L, and MCC.
Embodiment 5. The SDI formulation of any one of Embodiments 1-4, comprising about 25-40% wt compound A, or a pharmaceutically acceptable salt thereof.
Embodiment 6. The SDI formulation of any one of Embodiments 1-5, wherein the pharmaceutically acceptable polymer is about 60-75% wt.
Embodiment 7. The SDI formulation of any one of Embodiments 1-6, comprising 40:60 (wt %) compound A free base:HPMCAS-M.
Embodiment 8. A unit dosage form comprising the SDI formulation of any one of Embodiments 1-7.
Embodiment 9. The unit dosage form of Embodiment 8, wherein the SDI formulation is about 55-65 wt % of the unit dosage form.
Embodiment 10. The unit dosage form of Embodiment 8 or 9, which is an immediate release (IR) tablet.
Embodiment 11. The unit dosage form of any one of Embodiments 8-10, further comprising a filler selected from mannitol and lactose.
Embodiment 12. The unit dosage form of any one of Embodiments 8-11, further comprising a disintegrant Ac-Di-Sol.
Embodiment 13. The unit dosage form of any one of Embodiments 8-12, further comprising a thickening agent Cab-O-Sil.
Embodiment 14. The unit dosage form of any one of Embodiments 8-13, further comprising sodium stearyl fumarate.
Embodiment 15. The unit dosage form of any one of Embodiments 8-14, further comprising a binder HPC Nisso SSL SFP.
Embodiment 16. The unit dosage form of any one of Embodiments 8-15, which has a full release in about 3 minutes in a sink dissolution test.
Embodiment 17. A method for treating cancer in a patient, comprising administering to the patient a therapeutically effect amount of the SDI formulation of any one of Embodiments 1-7, or the unit dosage form of any one of Embodiments 8-16.
Embodiment 18. The method of Embodiment 17, wherein the cancer is bladder cancer.
Embodiment 19. The method of Embodiment 17, wherein the cancer is solid tumor.
Embodiment 20. The method of Embodiment 17, wherein the cancer is selected from a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.
Embodiment 21. The method of Embodiment 17, wherein the cancer is selected from urothelial carcinoma; head and neck squamous cell carcinoma; melanoma; ovarian cancer; renal cell carcinoma; cervical cancer; gastrointestinal/stomach (GIST) cancer; non-small cell lung cancer (NSCLC); acute myeloid leukemia (AML); and esophageal cancer.
Embodiment 22. The method of Embodiment 21, wherein the cancer is a urothelial carcinoma.
Embodiment 23. The method of Embodiment 22, wherein the urothelial carcinoma is bladder cancer.
Embodiment 24. The method of Embodiment 22, wherein the urothelial carcinoma is a transitional cell carcinoma.
Embodiment 25. The method of Embodiment 21, wherein the cancer is head and neck squamous cell carcinoma.
Embodiment 26. The method of Embodiment 21, wherein the cancer is a melanoma.
Embodiment 27. The method of Embodiment 26, wherein the melanoma is a uveal melanoma.
Embodiment 28. The method of Embodiment 21, wherein the cancer is ovarian cancer.
Embodiment 29. The method of Embodiment 28, wherein the ovarian cancer is a serous subtype of ovarian cancer.
Embodiment 30. The method of Embodiment 21, wherein the cancer is renal cell carcinoma.
Embodiment 31. The method of Embodiment 30, wherein the renal cell carcinoma is a clear cell renal cell carcinoma subtype.
Embodiment 32. The method of Embodiment 21, wherein the cancer is cervical cancer.
Embodiment 33. The method of Embodiment 21, wherein the cancer is a gastrointestinal/stomach (GIST) cancer.
Embodiment 34. The method of Embodiment 33, wherein the cancer is a stomach cancer.
Embodiment 35. The method of Embodiment 21, wherein the cancer is non-small cell lung cancer (NSCLC).
Embodiment 36. The method of Embodiment 35, wherein the NSCLC is advanced and/or metastatic NSCLC.
Embodiment 37. The method of Embodiment 21, wherein the cancer is esophageal cancer.
Embodiment 38. The method of any one of Embodiments 17-37, wherein the method comprises administering to the patient about 200-1600 mg of compound A, or a pharmaceutically acceptable salt thereof, daily.
Embodiment 39. The method of any one of Embodiments 17-38, wherein the method comprises administering to the patient about 200 mg of compound A, or a pharmaceutically acceptable salt thereof, daily.
Embodiment 40. The method of any one of Embodiments 17-38, wherein the method comprises administering to the patient about 400 mg of compound A, or a pharmaceutically acceptable salt thereof, daily.
Embodiment 41. The method of any one of Embodiments 17-38, wherein the method comprises administering to the patient about 600 mg of compound A, or a pharmaceutically acceptable salt thereof, daily.
Embodiment 42. The method of any one of Embodiments 17-38, wherein the method comprises administering to the patient about 800 mg of compound A, or a pharmaceutically acceptable salt thereof, daily.
Embodiment 43. The method of any one of Embodiments 17-38, wherein the method comprises administering to the patient about 1200 mg of compound A, or a pharmaceutically acceptable salt thereof, daily.
Embodiment 44. The method of any one of Embodiments 17-38, wherein the method comprises administering to the patient about 1600 mg of compound A, or a pharmaceutically acceptable salt thereof, daily.

### EXEMPLIFICATION

Compound A can be prepared by methods known to one of ordinary skill in the art, for example, as described in WO2018195397 and US20180327411, the contents of which are incorporated herein by reference in their entireties.

### List of Abbreviations

AE adverse event
AHR aryl hydrocarbon receptor
ALP alkaline phosphatase
ALT alanine aminotransferase
ANC absolute neutrophil count
aPTT activated partial thromboplastin time
ARNT aryl hydrocarbon receptor nuclear translocator
AST aspartate aminotransferase
AUC area under the plasma concentration-time curve
AUC₀₋₂₄ area under the plasma concentration-time curve from time 0 to 24 hours
BCRP breast cancer resistance protein
BID twice a day
BOR best overall response
C#D# cycle number day number
CI confidence interval
CL clearance
Cₘₐₓ maximum observed {plasma/blood/serum} concentration
CNS central nervous system
CR complete response
CSR clinical study report
CT computed tomography
CYP cytochrome
DCR disease control rate
DLT dose-limiting toxicity
DOR duration of response
DOT duration of treatment
DRE dioxin response elements
ECG electrocardiogram
ECI events of clinical interest
ECOG Eastern Cooperative Oncology Group
eCRF case report form (electronic or paper)
EOS end of study
EOT end of treatment
ET early termination
FDA Food and Drug Administration
FDG fluoro-2-deoxyglucose
FIH first-in-human
FSH follicle stimulating hormone
GCP Good Clinical Practice
G-CSF granulocyte colony-stimulating factor
GI gastrointestinal
GFR glomerular filtration rate
GLP Good Laboratory Practice
GM-CSF granulocyte-macrophage colony-stimulating factor
HED human equivalent dose
HIV human immunodeficiency virus
HRT hormone replacement therapy
HNSTD highest non-severely toxic dose
IB Investigator Brochure
IC₅₀ half maximal inhibitory concentration
ICF informed consent form
ICH International Council for Harmonisation
IDO1 indoleamine 2, 3-dioxygenase
IEC Institutional Ethics Committee
IL interleukin
INR international normalised ratio
irAE immune-related adverse event
iRECIST immune Response Evaluation Criteria in Solid Tumors
IRB institutional review board
IV intravenous(ly)
LLN lower limit of normal
LV left ventricular
LVEF left ventricular ejection fraction
MedDRA Medical Dictionary for Regulatory Activities
MRI magnetic resonance imaging
MTD maximum tolerated dose
mTPI modified Toxicity Probability Interval trial design
mTPI-2 Revision of modified Toxicity Probability Interval trial design
NCI-CTCAE National Cancer Institute Common Terminology Criteria for Adverse Events
NLNT new lesions non-target
NLT new lesions-target
NSAIDs nonsteroidal anti-inflammatory drugs
ORR objective response rate
PCR polymerase chain reaction
PD progressive disease
PD-1 programmed cell death 1
PET positron emission tomography
PFS progression-free survival
PK pharmacokinetics
PO orally
PR partial response
PT prothrombin time
q8h every 8 hours
q12h every 12 hours
q4w every 4 weeks
QD once daily
QID four times a day
QTcF QT interval corrected by the Fridericia's Correction formula
RECIST 1.1 Response Evaluation Criteria in Solid Tumors version 1.1
RP2D recommended phase 2 dose
SAE serious adverse event
SD stable disease
SAP statistical analysis plan
SoE Schedule of Events
SRM study reference manual
STD10 Severely toxic dose to 10% of animals
SRC Safety Review Committee
SUSAR suspected unexpected serious adverse reactions
t_{1/2} half-life
TDO2 tryptophan 2,3-dioxygenase 2
TEAE treatment-emergent adverse event
TID three times per day
Tregs regulatory T cells
ULN upper limit of normal
Vss steady state volume of distribution
WHO World Health Organization
WOCBP women of child-bearing potential

### Example 1. Preparation of Compound A Formulations and Unit Dosages

### 1. Materials and Methods

The formulations and unit dosages are prepared using Compound A FB or hemi-maleate salt.

Potential excipients for spray dried solid dispersions and tablet development and manufacturing were of compendial or USP grade. A full list of excipients and equipment utilized for this body of work are listed below. Percent compositions of solutions or solid dispersions are described on a weight:weight basis, unless otherwise specified.

TABLE 1
Materials and Equipment

| | | | |
|---|---|---|---|
| Material and Equipment | Trade Name or Model | Abbreviation | Manufacturer |

Materials

| | | | |
|---|---|---|---|
| Acetone | Acetone | Acetone | Fisher |
| Methanol | Methanol | MeOH | Fisher |
| Ethanol | Ethanol | EtOH | Sigma-Aldrich |
| Methylene Chloride | Methylene Chloride | DCM | Fisher |
| Ethyl Acetate | Ethyl Acetate | EtOAc | Fisher |
| Polyvinylpyrrolidone/Vinyl | Kollidon VA 64 | PVP-VA | BASF |
| acetate copolymer | | | |
| Hypromellose | Methocel | HPMC | DOW |
| Hypromellose phthalate | Hypromellose phthalate | HPMCP-55 | Shin Etsu |
| Hypromellose acetate | AQOAT-MG | HPMCAS-M | Shin Etsu |
| succinate MG grade | | | |
| Hypromellose acetate | AQOAT-LG | HPMCAS-L | Shin Etsu |
| succinate LG grade | | | |
| Vitamin E TPGS | TPGS | TPGS | Antares |
| Microcrystalline Cellulose | Avicel PH-105 | MCC | FMC Biopolymer |
| Microcrystalline Cellulose | Avicel PH-200 | MCC | FMC Biopolymer |
| Lactose Monohydrate, | FlowLac 90 | NA | Meggle |
| Spray Dried | | | |
| Croscarmellose Sodium | Ac-Di-Sol | Ac-Di-Sol | FMC Biopolymer |
| Colloidal Silicon Dioxide | Cab-O-Sil | Cab-O-Sil | Cabot Corporation |
| Sodium Stearyl Fumarate | Pruv SSF | SSF | JRS Pharma |
| Mannitol | Pearlitol 100 SD | Mannitol | Roquette |
| Crospovidone | Kollidon CL-F | Kollidon CL-F | BASF |

### 2. Methods

Compound A free base and hemi-maleate salt, subsequent spray dried intermediates (SDIs), and tablets were characterized using one or more of the following analytical experiments: modulated differential scanning calorimetry (MDSC), X-ray powder diffraction (XRPD), residual solvents by gas chromatography headspace sampling (GC-HS), scanning electron microscopy (SEM), polarized light microscopy (PLM), assay and impurities by high performance liquid chromatography (HPLC), water content by Karl Fisher titration (KF), dynamic vapor sorption (DVS), and non-sink dissolution.

### 2.1. Differential Scanning Calorimetry (DSC)

DSC was performed using a TA Instruments Discovery DSC2500 differential scanning calorimeter equipped with a TA instruments Refrigerated Cooling System 90 operating in either modulated or ramp mode. DSC was used to measure thermodynamic events and characteristics of Compound A free base and hemi-maleate salt, and subsequent SDIs. Events observed include the glass transition temperature (Tg), cold crystallization (Tc), defined as a crystallization event at a temperature lower than the melt temperature, and melting temperature (Tm). SDI samples were placed in non-hermetic aluminum pans and heated at a constant rate of 2.0° C./min over a 5-200° C. temperature range. The system was purged by nitrogen flow at 50 mL/min to ensure inert atmosphere through the course of measurement. Compound A free base and hemi-maleate salt was initially analyzed by standard DSC with a heating rate of 10° C./min ramping up to 215° C. for compound A free base and 185° C. for compound A hemi-maleate salt. Amorphous API was successfully created by rapidly quenching liquefied Compound A using Refrigerated Cooling System 90. The resulting amorphous API was analyzed by modulated DSC. A summary of DSC analysis parameters can be found in Tables 2 and 3.

TABLE 2
DSC Analysis Parameters

| | |
|---|---|
| Instrument | TA Discovery DSC2500, RCS 90 |
| Sample Pans | Tzero Al, Non-hermetic |
| Temp. Range | 0-185° C. (Free base) |
| | 0-215° C. (Hemi-maleate salt) |
| Heating Rate | 10° C./min |
| Scanning Mode | Ramp |

TABLE 3
MDSC Analysis Parameters

| Instrument | TA Discovery DSC2500, RCS 90 | |
|---|---|---|
| Sample Pans | Tzero Al, Non-hermetic | |
| Temp. Range | 0-200° | C. |
| Heating Rate | 2° | C./min |
| Scanning Mode | Modulated | |
| Modulation Frequency | 60 | s |
| Modulation Amplitude | 1° | C. |

### 2.2. X-ray Powder Diffraction (XRPD)

XRPD was performed using a Rigaku Miniflex 6G X-ray diffractometer to evaluate the crystallinity of spray dried materials. Amorphous materials give an “amorphous halo” diffraction pattern, absent of discrete peaks that would be found in a crystalline material. Samples were irradiated with monochromatized Cu Kα radiation and analyzed between 5° and 400 with a continuous scanning mode. Samples were rotated during analysis to minimize preferred orientation effects. A summary of XRPD analysis parameters can be found in Table 4.

TABLE 4
XRPD Analysis Parameters

| Instrument | Rigaku Minifiex 6G | |
|---|---|---|
| Radiation Source | Cu-Kα (1.5406 Å), Line Focus 0.4 mm × 12 mm | |
| Scan Type | Coupled 2θ/θ | |
| Scan Range | 5°-40° | |
| Step Increment | 0.005° | |
| Ramp Rate | 0.37 | min |
| Voltage | 40 | kV |
| Current | 15 | mA |
| Rotation | 30 | rpm |
| Holder | Zero-Background Cup | |
| Divergent Slit Width | 0.625 | mm |

### 2.3. Particle Morphology by Scanning Electron Microscope (SEM)

SEM samples were prepared by dispersing powder onto an adhesive carbon-coated sample stub and coating with a thin conductive layer of gold using a Cressington 108 Auto. Samples were analyzed using a FEI Quanta 200 SEM fitted with an Everhart-Thornley (secondary electron) detector operating in high vacuum mode. Micrographs at various magnifications were captured for qualitative particle morphology analysis. Experimental parameters including spot size, working distance, and acceleration voltage were varied from sample to sample to obtain the best imaging conditions, and are documented in the caption of each SEM micrograph.

### 2.4. Particle Size Distribution (PSD) by Light Diffraction

The particle size distribution of SDI samples was determined by laser diffraction using a Mastersizer 3000 with an Aero S unit (Malvern Instruments). About 100 mg samples were added to the standard venture disperser with a hopper gap of 1.0 mm and then fed into the dispersion system. The feed rate of 80-90% was adjusted to keep the laser obscuration level at 0.1-10.0%. Compressed air at 2.0 bar was used to transport and suspend the sample particles through the optical cell. A measurement time of 5 seconds was used, and background measurements were made using air for 10 seconds. Dv10, Dv50 and Dv90 diameters are used to characterize the particle size distribution of powders. For instance, the Dv50 is the diameter at which 50% of a sample's volume is comprised of smaller particles.

### 2.5. Assay and Impurities Analysis by HPLC

Assay and impurities of SDI samples were evaluated using an experimental HPLC method (Table 5). The method demonstrated a linear response, selectivity, and separation of previously seen impurities. The RT for Compound A is about 15.1 minutes.

TABLE 5
HPLC Parameters for Assay and Impurities Analysis

| Column | Agilent Zorbax SB-Phenyl, 4.6 × 150 mm, | | |
|---|---|---|---|
| | 3.5 μm, PN: 863953-912, SN: USNF014175 | | |
| Mobile Phase A | 0.05% TFA in H₂O (v/v) | | |
| Mobile Phase B | 0.05% TFA in Acetonitrile (ACN) (v/v) | | |
| Diluent | 3:1 ACN:H₂O (v/v) | | |
| Program Type | Gradient | | |
| | Time | % Mobile | % Mobile |
| Gradient Program | (min) | Phase A | Phase B |
| | 0.0 | 85 | 15 |
| | 3.0 | 65 | 35 |
| | 8.0 | 45 | 55 |
| | 16.0 | 5 | 95 |
| | 21.0 | 5 | 95 |
| | 21.1 | 85 | 15 |
| | 30.0 | 85 | 15 |

| | |
|---|---|
| Flow Rate | 1.0 mL/min |
| Column Temperature | 30° C. |
| Sample Temperature | Room temp. |
| Injection Volume | 5 μL |
| Needle Wash | Diluent |
| Detection Method | UV |
| Detection Wavelength | 230 nm |
| Detection Bandwidth | 8 nm |
| Slit Width | 16 nm |
| Reference Wavelength | Off |
| Collect Spectra | ~190-400 nm |
| Run Time | 30 min |

### 2.6. Residual Solvent by Gas Chromatography Headspace Sampling

The residual solvent contents of the SDIs were measured by GC-HS after secondary drying. Measurements were made using an HP 6890 series GC equipped with an Agilent 7697A headspace sampler. A 30 m×0.32 mm×1.8μ capillary column with 6% cyanopropylphenyl 94% dimethylpolysiloxane GC column was used for the testing. GC samples were prepared by dissolving ˜100 mg sample in 4 mL dimethyl sulfoxide (DMSO). GC-HS method (DM-123) was used for the drug product at this stage. The GC method parameters are summarized in Table 6.

TABLE 6
GC-HS Method Parameters

| Sample Temperature | 105° | C. |
|---|---|---|
| Sample Loop Temperature | 110° | C. |
| Transfer Line Temperature | 115° | C. |
| GC Cycle Time: | 30 | min |
| Vial Equilibration Time: | 30 | min |
| Injection Time: | 1.00 | min |
| Injection Loop Size: | 1 | mL |
| Post Injection Purge | 100 mL/min; 1 min | |
| Carrier Gas: | N₂, ≥99.999% | |
| Carrier Gas Flow | 25 | mL/min |
| Vial Pressure | 15.0 | psi |

### 2.7. Intrinsic Dissolution Performance

Milled and as-received API samples were weighed and compressed into a compact using a hydraulic press at 3,000 psi for 60 seconds. Compacts were mounted on intrinsic dissolution apparatus and dissolution study was conducted using USP dissolution apparatus in 250 mL of 0.5% Tween 80 solution at 100 rpm at 37° C. in duplicate. Aliquots (1.0 mL) of dissolution media are taken at selected time-points from each dissolution vessel at 5 minute intervals from 5 to 40 minutes. Each sample was centrifuged at 14,000 rpm for 3 minutes and the supernatant was samples and diluted with diluent for HPLC analysis (Table 7). The retention time of Compound A is about 1.5 minutes.

TABLE 7
HPLC Parameters for Dissolution Analysis

| Column | Phenomenex Kinetex Phenyl-Hexyl, | |
|---|---|---|
| | 4.6 × 50 mm, 2.7 μm, PN: 00B-4495-E0 | |
| Mobile Phase A | 0.1% Trifluoroacetic acid (TFA) in | |
| | H₂O (v/v) | |
| Mobile Phase B | 0.1% TFA in ACN (v/v) | |
| Diluent | 3:1 Acetonitrile (ACN):H₂O (v/v) | |
| Isocratic Program | MPA (%) | MPB (%) |
| | 30 | 70 |

| | |
|---|---|
| Flow Rate | 1.0 mL/min |
| Column Temperature | 40° C. |
| Sample Temperature | Ambient |
| Injection Volume | 12 μL |
| Detection Method | UV |
| Detection Wavelength | 278 nm |
| Detection Bandwidth | 4 nm |
| Slit Width | 4 nm |
| Reference Wavelength | Off |
| Run Time | 3 min |

### 2.8. Non-Sink Dissolution Performance

In vitro drug dissolution performance for both API forms and each SDI was evaluated by the two stage ‘gastric transfer’ non-sink dissolution test (Table 8), which simulates pH and bile salt concentrations for both gastric and intestinal exposure in a sample to perform assay. Pre-weighed SDI powder is briefly suspended in media (e.g., by 10 sec vortex mixing with 4.0 mL media) and transferred to a pre-heated (37° C.) volume of 50 mL of 0.1N HCl (aq) (simulated gastric fluid or SGF, pH ˜1.0, without pepsin or bile salts), in a USP Type 2 mini-vessel (100 mL total vessel volume) while stirring (paddles) at 100 rpm. After 30 minutes of gastric pH exposure, an equal volume of PBS buffered, 2× concentrated fasted-state simulated intestinal fluid (FaSSIF) is added to the SGF, resulting in a final pH of 6.8 in FaSSIF (100 mM PBS containing 2.24 mg/mL SIF powder (original) (Biorelevant Inc.) in a total volume of 100 mL. Aliquots (1.0 mL) of dissolution media are taken at selected time-points before and after the simulated gastric transfer, spundown (13,000 rpm) to pellet out undissolved solids, and the supernatant sampled and further diluted in an appropriate diluent to determine API total drug concentration (e.g. free and colloidal/polymer-bound drug in solution) utilizing a suitable HPLC method. The volume of FaSSIF added is adjusted to account for the sampling volume removed prior to gastric transfer (typically 4×1.0 mL). Initial Compound A API measurements and SDI dissolution samples were determined utilizing a HPLC method (Table 7).

TABLE 8
Non-Sink Dissolution Test Parameters

| Apparatus | DSP Type 2 (100 mL) | |
|---|---|---|
| Gastric Media | 0.1N HCl (aq) | |
| Intestinal Media | FaSSIF | |
| Temperature | 37 ± 0.5° | C. |
| Paddle Speed | 100 | RPM |
| Dose | 1.0 → 0.5 | mgA/mL |

### 2.9. Preparation of Dissolution Media

0.5% (w/w) Tween 80 in Water: Determine the weight of Tween 80 needed for all dissolution samples. Based on this weight, weigh out suitable amount of Tween 80 into a suitable Class A beaker and add 10% volume of water to dissolve Tween 80. Transfer the rest of water into the beaker and mix well.

Simulated Gastric Fluid (SGF): Determine the volume of SGF needed for all dissolution samples. Based on this volume, dilute 1.0N HCl 10× with H2O in a suitable Class A graduated cylinder or volumetric flask. Mix well, test approximate pH using pH paper. The observed pH should be 1.0-1.1.

PBS buffer (200 mM): Determine the volume of buffer needed for all dissolution samples. Based on this volume, weigh 200 mMol/L NaCl and 200 mMol/L Na2HPO4 and transfer into an appropriately-sized vessel. To this vessel, add the appropriate volume of H2O. Sonicate the solution until all salts are fully dissolved. If necessary, adjust with phosphoric acid or 1.0N NaOH to pH 8.9±0.1.

FaSSIF Media (4.48 mg/mL): To PBS media above, add 4.48 mg SIF powder per mL of 200 mM PBS. Mix well, stirring with a magnetic stir bar until all SIF is in solution. Let stand two hours at RT before use, and then pre-heat to 37° C. for the dissolution test. If FaSSIF will not be used the day it is prepared, store in refrigerator (2-8° C.) for up to 4 days. Remove from refrigerator at least two hours before use, ensuring that the solution reaches 37° C. prior to use.

### 2.10. Bulk Density and Tapped Density Analysis

Tablet blends were evaluated for bulk and tapped density per USP <616> “Tapped Density—Method I”. A 100-mL glass cylinder along with corresponding base plate was used for all samples. An ERWEKA SVM Tapped Density Tester was utilized to perform analysis and tapped at a rate of 300 taps/minute.

### 2.11. Tablet Friability

Tablet friability was determined by USP <1216> utilizing a Pharmatron FT 2 friability tester. A drum rotation speed of 25 rpm was used at a total rotation time of 4 minutes. Acceptable loss on friability per USP method is ≤1.0 weight percent.

### 2.12. Disintegration

Disintegration was evaluated per USP <701> “Disintegration” utilizing a Varian VK-100 disintegration apparatus. The apparatus consists of a 1000 mL low-form beaker and basket-rack assembly with six open-ended transparent tubes. The beaker contained 750 mL of RO water and was maintained at a temperature of 37° C. (±2° C.). The basket was fully submerged at a frequency of 29-32 cycles per minute and tablet disintegration time was recorded when the last visible tablet materials passed through the basket.

### 2.13. Tablet Hardness and Tensile Strength

Tablet hardness was tested per USP <1217> “Tablet Breaking Force” utilizing a Natoli Hardness Tester (S/N 1403029). Tablet thickness and weight were measured prior to assessing the tablet break force as it is a destructive process. Tablets were placed in the automated breaking apparatus and tablet hardness was measured in kilogram-force/kilopond (kp).

Tensile strength for standard round concave (SRC) tablets was calculated based on the following equation:

$$Tablet Tensile Strength ( S R C ) = \frac{10 P}{π {D}^{2} ( 2.84 \frac{t}{D} - 0.126 \frac{t}{W} + 3.15 \frac{W}{D} + 0.01 )}$$

Where P=fracture load, D=tablet width, t=tablet thickness, W=band thickness (K. G. Pitt and M. G. Heasley. “Determination of the tensile strength of elongated tablets.” Powder Technology, vol. 238 (2013) pp. 169-175.)

### 2.14. Jet-Milling

In an attempt to increase the bioavailability, both the Compound A FB API and hemi-maleate salt form of the API were subjected to particle size reduction using air-jet micronization technique (aka, jet-milling). Jetmilling of the API would increase the surface-area-to-volume ratio resulting in increased exposure. The manufacturing process for jet-milled API is described in the flow diagram below and the manufacturing parameters are listed in Table 9.

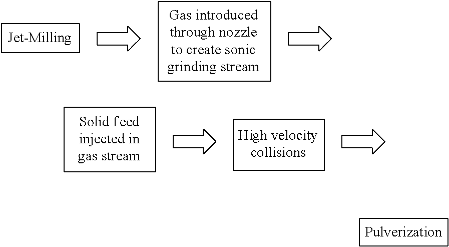

TABLE 9
Parameters and yields for jet-milling of
bulk Compound A FB and Hemi-maleate salt

| Parameters | API | Yield | Observation |
|---|---|---|---|
| Pusher Nozzle Pressure: | Compound A FB | 85.17 | Free flowing |
| 80 psi | | | (no static) |
| Grinder Nozzle Pressure: | Compound A | 64.35 | Material sticky |
| 100 psi | Hemi-maleate salt | | (static) |

### 2.15. Particle Size Distribution by Sieve Analysis

Particle size distribution was determined by an analytical sieving method similar to USP <786>. A RO-TAP RX-29-E sieve shaker (W. S. Tyler) was utilized to evaluate material. Screens utilized and operating parameters can be found below in Table 10.

TABLE 10
Equipment and Parameters for Particle Size Distribution
Analysis via Analytical Sieving Method

| Equipment Manufacture | W. S. Tyler | |
|---|---|---|
| Sieve Shaker Model | RO-TAP RX-29-E | |
| Shaker Mode | Coarse | |
| Operating Time | 5 min | |
| Screen #1 | 20 mesh | (841 μm) |
| Screen #2 | 30 mesh | (595 μm) |
| Screen #3 | 40 mesh | (420 μm) |
| Screen #4 | 60 mesh | (250 μm) |
| Screen #5 | 120 mesh | (125 μm) |
| Screen #6 | 200 mesh | (74 μm) |

### 3. Results and Discussion

### 3.1. Compound Analysis and Property Assessment

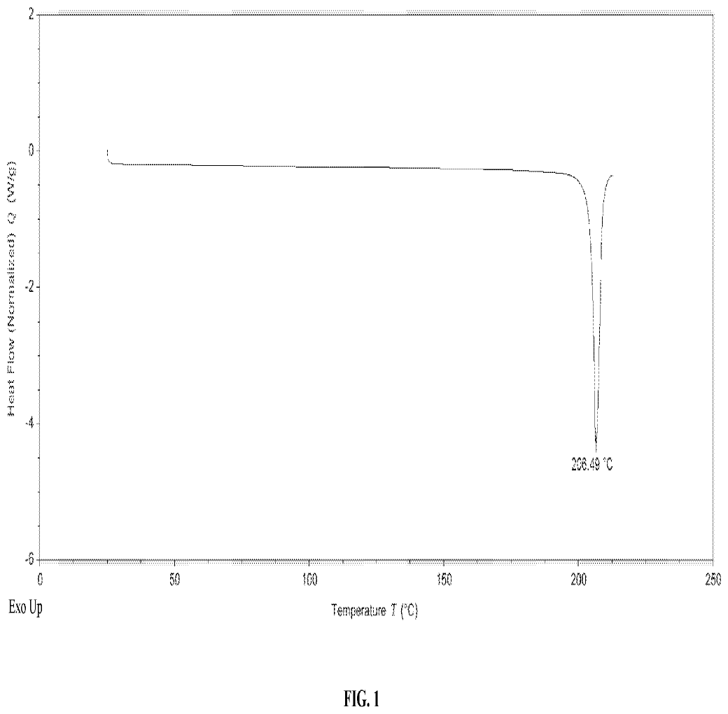

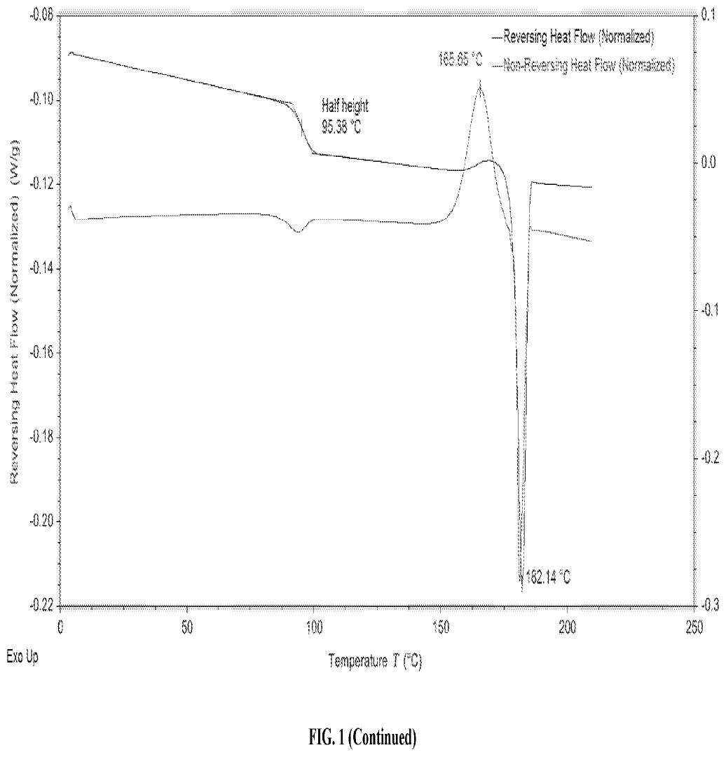

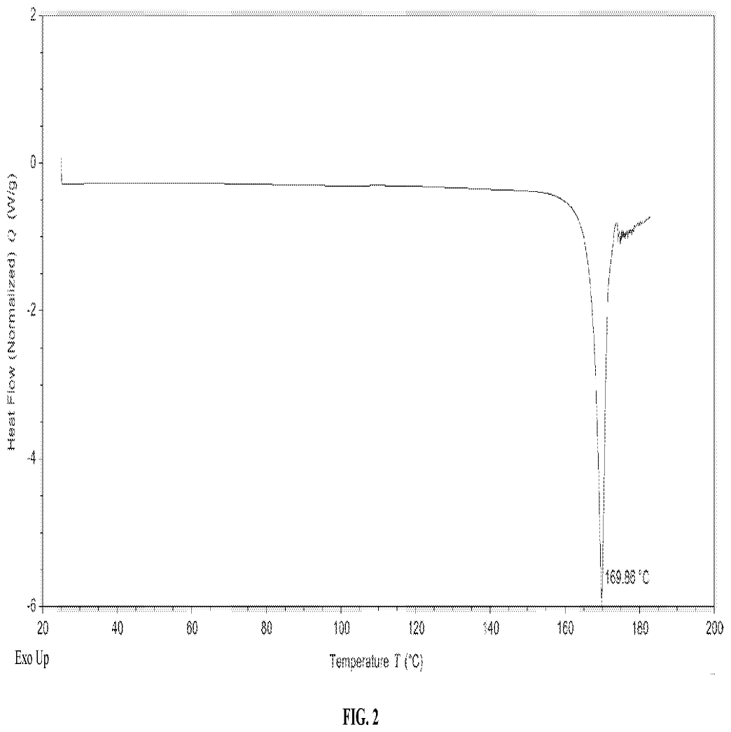

Thermal properties of both Compound A forms were measured by both DSC and MDSC. During standard mode ramping experiments, sharp endothermic melting events (Tm) were observed at 206° C. for the free base, and 170° C. for the salt form with decomposition observed in the liquid phase. The Tg of each was measured via a melt-quench technique, heating past its melting temperature and rapidly cooling to trap the molten material in an amorphous state. The resulting samples were analyzed by MDSC and a Tg of 95° C. was observed for the free base, with a clear crystallization peak at 165° C. and a melting event at 182° C., indicating the conversion to a different polymorph (FIG. 1/Table 11). The hemi-maleate salt displayed signs of possible degradation, showing a broader glass transition and noisier baseline with no crystallization or melting observed up to 180° C., with a Tg measured at 83° C. (FIG. 2/Table 11). This results in a Tm/Tg ratio of 1.30 and 1.24 for the free base and salt forms respectively, indicative of moderate physical stability.

TABLE 11
Summary of Thermal Properties of Compound A Free Base

| Material | Tg | Tc | Tm |
|---|---|---|---|
| Compound A Free Base | 95° C. | 166° C. | 206° C. |
| Compound A Hemi-Maleate Salt | 83° C. | NA | 169° C. |

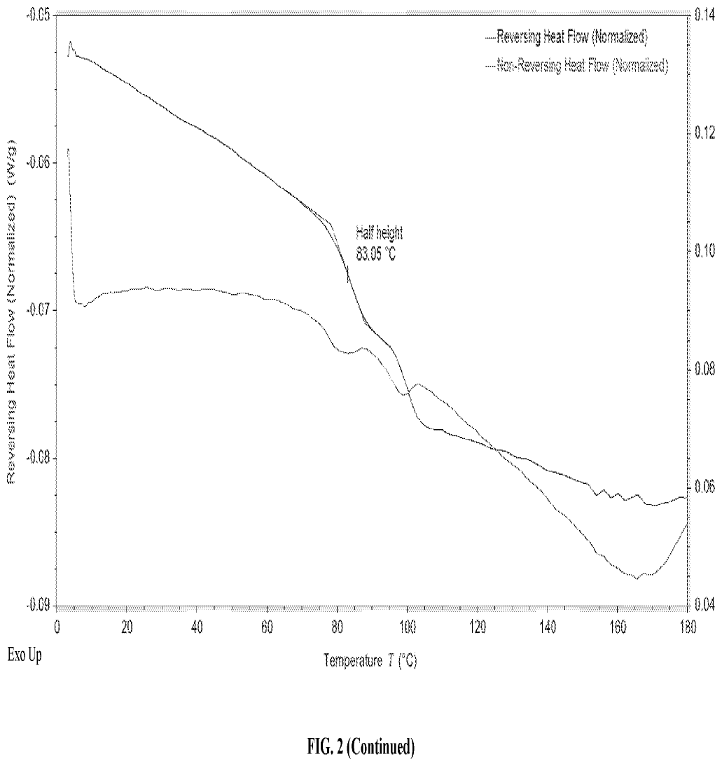

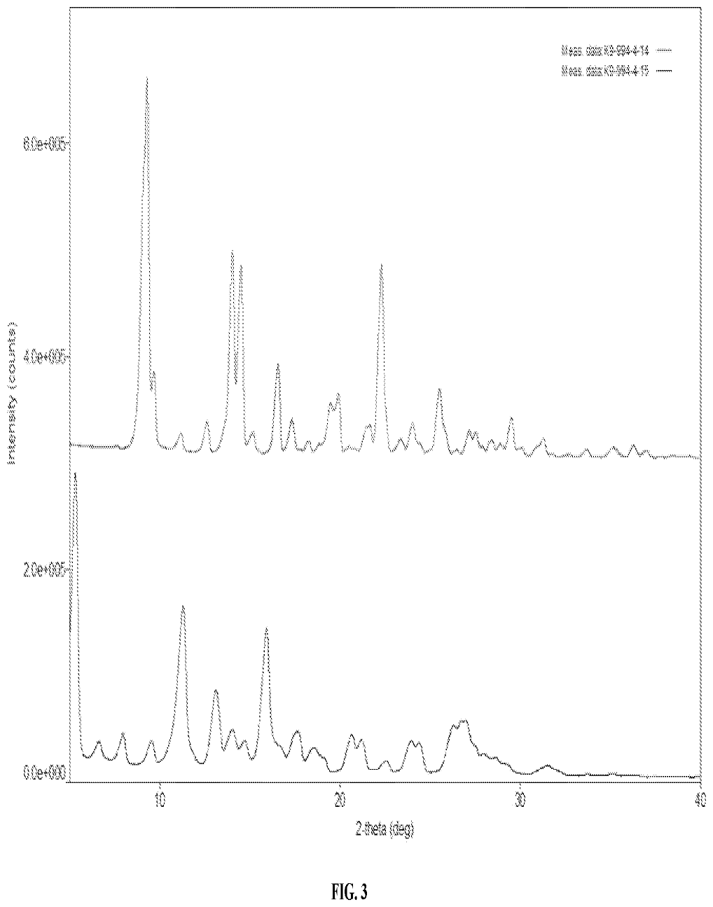

A diffraction pattern of both forms of crystalline Compound A was performed using XRPD (FIG. 3). The unique diffraction patterns of the bulk APIs indicate a crystalline material, consistent with thermal analysis.

Surface morphology of both forms of the bulk API particles was characterized using scanning electron microscopy (the SEM images are not provided). Free base API morphology consists of high aspect ratio columnar orthogonal particles with drusy, while the maleate salt has a similar particle morphology only smaller and agglomerated.

### 3.2. Organic Solubility

Organic solubility of Compound A FB and the hemi-maleate salt form was determined visually in common spray drying solvents (Table 12). Free Base API demonstrated solubility higher than 2.0% in acetone and less than 2.0% solubility in the other solvents tested. The hemi-maleate salt was highly soluble in acetone (between 6.00-7.50%). Acetone was selected as the spray drying solvent based on sufficient API solubility as well as the ICH limit of residual acetone (i.e., Class 3 solvent, 5,000 ppm).

TABLE 12
Solubility of Bulk Compound A in Organic
Solvents Determined Visually

| | Compound A Free | |
|---|---|---|
| | Base Visual | Compound A hemi-maleate |
| Solvent System | Solubility, S (wt. %) | Visual Solubility, S (wt. %) |
| Acetone | 2.0 ≤ S < 3.0 | 6.0 ≤ S < 7.5 |
| 1:1 DCM:MeOH | 1.5 ≤ S < 2.0 | ND |
| EtOAc | 1.0 ≤ S < 2.0 | ND |
| 1:1 Ethanol:Acetone | 1.5 ≤ S < 2.0 | ND |
| 1:1 MeOH:Acetone | S < 1.5 | ND |
| DCM | ND | Not soluble |
| MeOH | ND | Not soluble |
| 80:20 DCM:MeOH | ND | 10.0 ≤ S < 12.0 |
| Ethanol | ND | Not soluble |

*ND = not determined

### 3.3. Aqueous Solubility

Solubility of bulk APIs was conducted in various biorelevant media. Small amounts of API were suspended in media and continuously agitated at room temperature for a period up to 24 hours. Samples were centrifuged to pellet out undissolved solids and the resulting supernatant was sampled, diluted, and analyzed by HPLC utilizing the short-assay method sued for dissolution sample analysis. Results are listed below in Table 13. Both forms show poor gastric solubility, with the maleate salt displaying an order of magnitude larger solubility in intestinal media.

TABLE 13
Solubility of Bulk Compound A Free Base and Maleate
Salt in Biorelevant Media Measured by HPLC

| Material | Media | Solubility (μg/mL) |
|---|---|---|
| Compound A Free Base | 0.1N HCl | <0.5 |
| | FaSSIF, pH 6.8 | 1.4 |
| Compound A maleate salt | 0.1N HCl | <0.5 |
| | FaSSIF, pH 6.8 | 50.9 |

### 3.4. Jet-Milled API Characterization

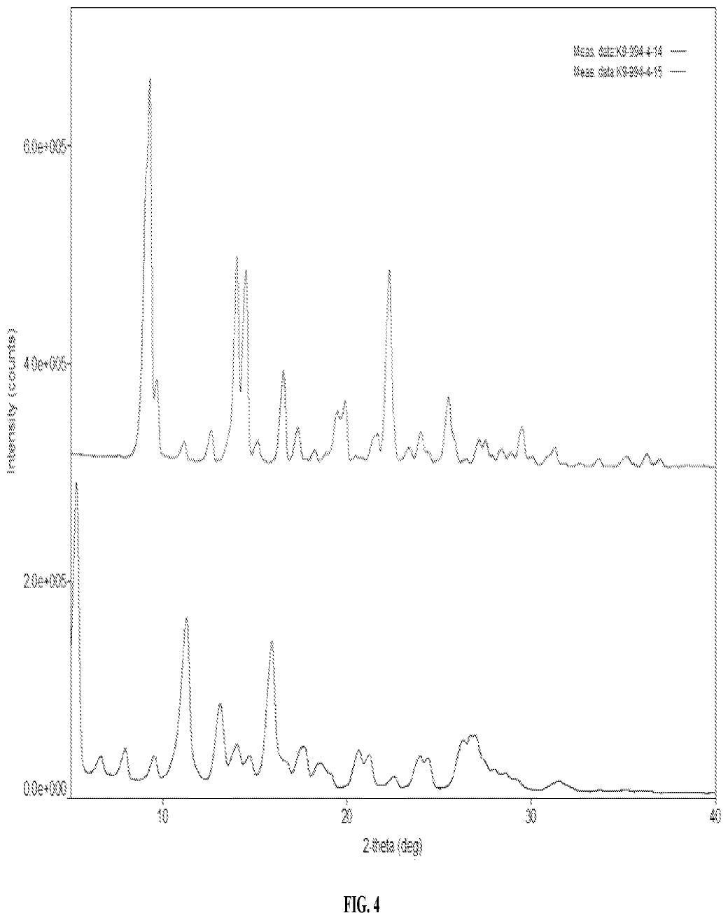

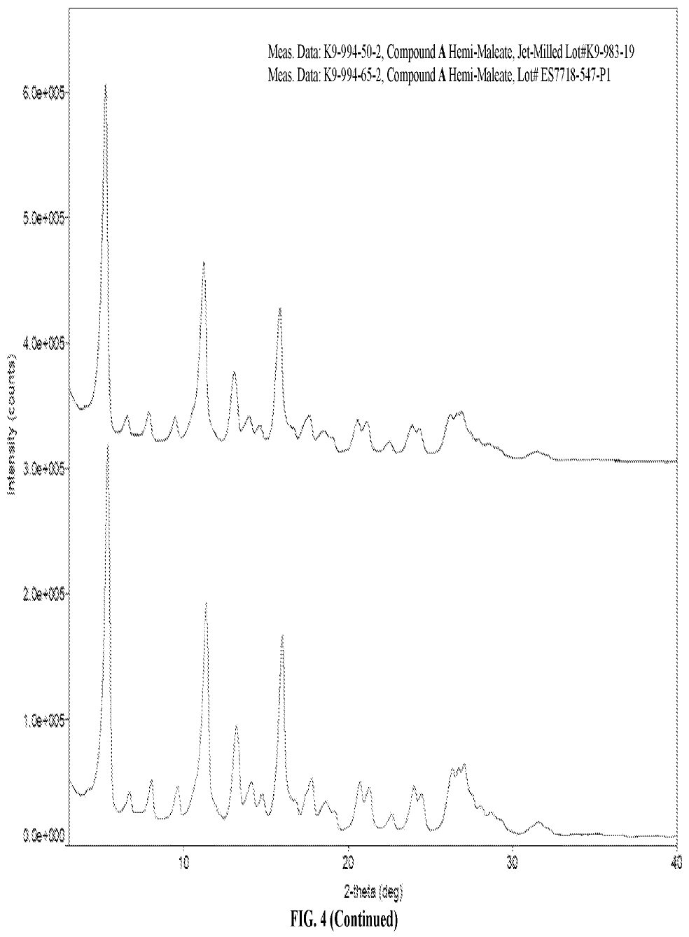

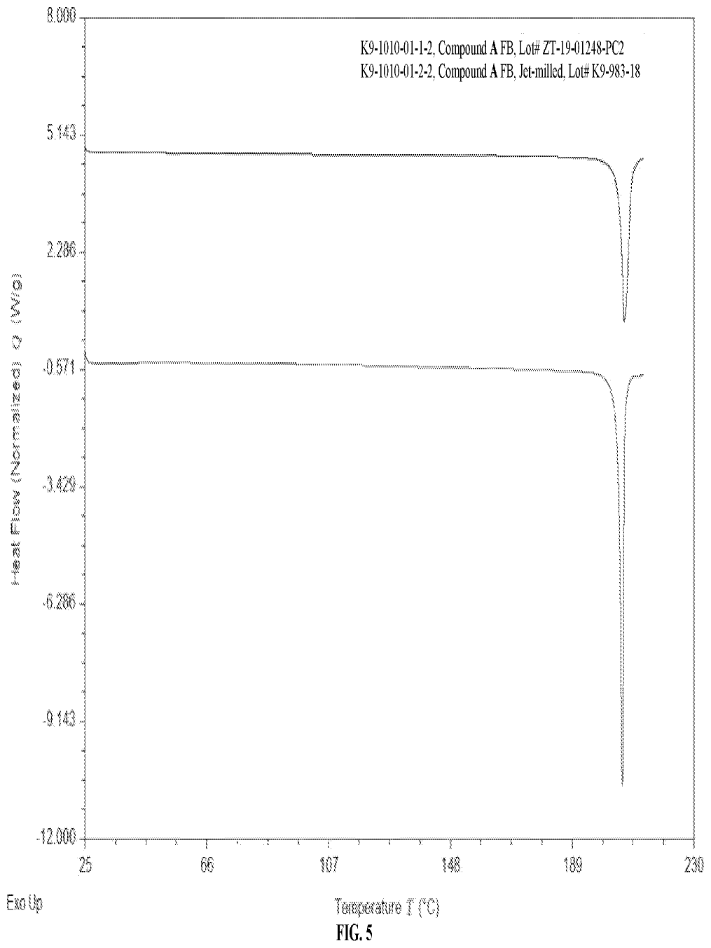

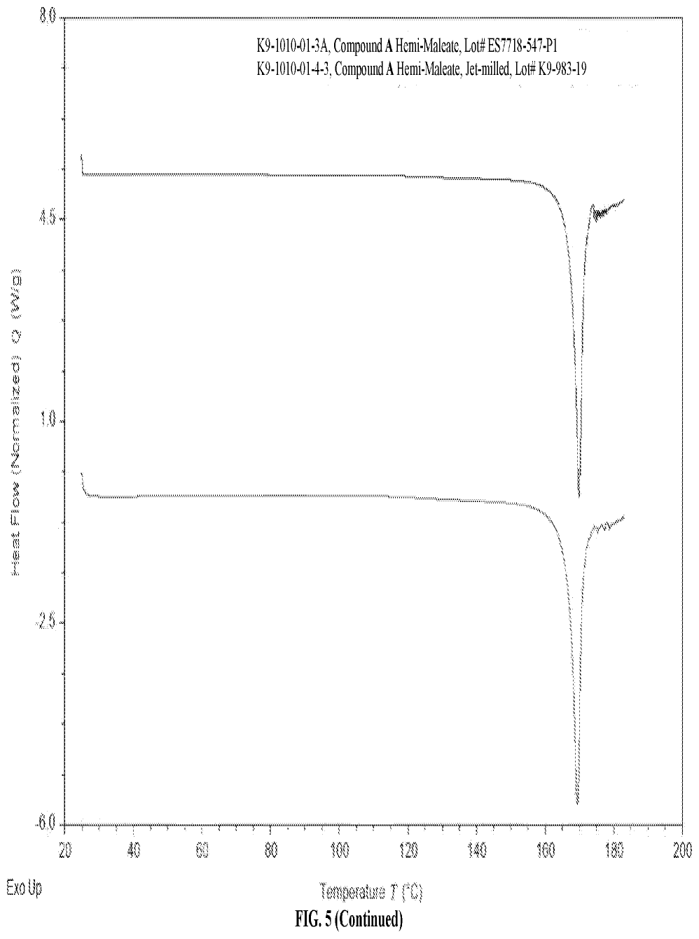

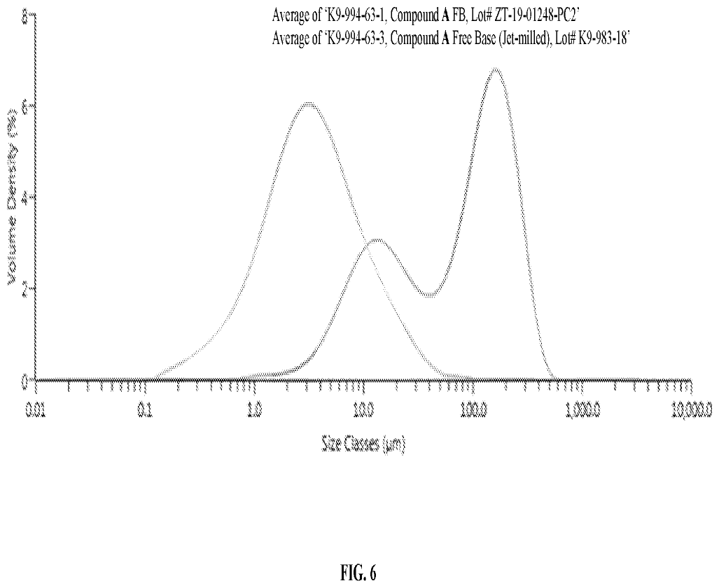

Results of jet-milling do not appear to have impacted the crystal form or thermodynamic properties of Compound A free base or maleate salt (FIGS. 4 and 5). SEM micrographs show a clear reduction in particle sizes for both forms, as well as PSD data, which shows both a decrease in size distribution, and also the removal of a bi-modal distribution, FIGS. 6-7, and Table 14 below.

TABLE 14
PSD of Compound A Free Base, Maleate Salt,
and Subsequent Jet-Milled Material

| | Dx (10) | Dx (50) | Dx (90) |
|---|---|---|---|
| Material | (μm) | (μm) | (μm) |
| Compound A Free Base | 8.27 | 88.0 | 245.0 |
| Compound A Hemi-Maleate salt | 3.25 | 18.4 | 213.0 |
| Compound A Free Base (Jet-Milled) | 0.83 | 3.3 | 13.0 |
| Compound A Hemi-Maleate salt (Jet-Milled) | 0.62 | 1.8 | 9.0 |

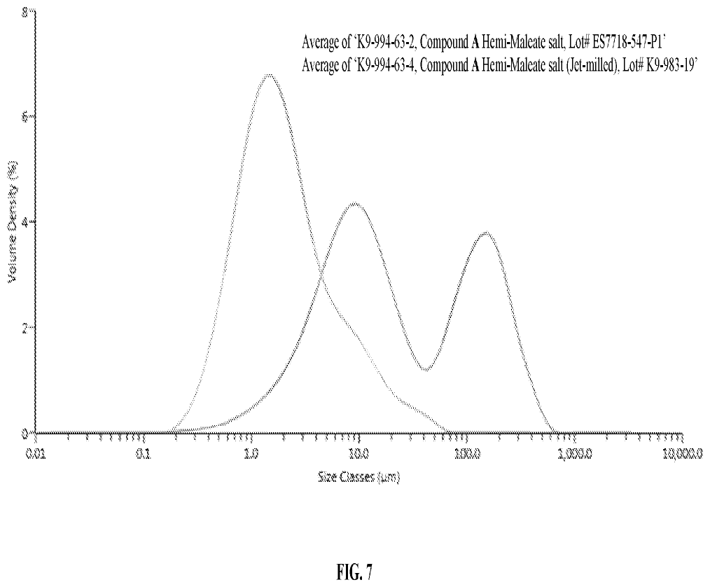

The Intrinsic dissolution performance of the jet-milled API vs. as received API was conducted to observe any increase in dissolution by particle size reduction. FIG. 8 and Table 15 below show the intrinsic dissolution results. The jet-milling process did not increase the intrinsic dissolution rate for either API form, indicating particle size reduction is not a viable path forward to increasing bioavailability of Compound A. The maleate salt outperformed the free base considerably due to its increased solubility in the media, 0.5% Tween 80 (aq).

TABLE 15
Intrinsic Dissolution Data of Compound A Free Base,
Maleate Salt, and Subsequent Jet-Milled Material

| Sample | IDR (ug/min*cm²) | R² |
|---|---|---|
| Compound A Hemi-Maleate salt | 2.63 | 0.99 |
| Compound A Hemi-Maleate salt (Jet-Milled) | 2.44 | 1.00 |
| Compound A Free Base | 0.85 | 0.89 |
| Compound A Free Base (Jet-Milled) | 0.57 | 0.96 |

### 3.5. Computational (In Silico) Modeling

Molecular modeling activities were performed utilizing the Quadrant 2® platform to evaluate specific drug-drug and drug-polymer interactions for Compound A. Modeling methods ranged from high level quantum mechanics calculations to molecular mechanics and molecular dynamics using a suite of programs. The goals of this work were to examine the drug-drug and drug-polymer molecular level interactions between Compound A and compendial GRAS polymers in order to provide a rational basis for selection of appropriate polymers for inclusion in a solubilized drug product intermediate. This rationale is based on molecular descriptors and specific drug-polymer interaction energies.

### 3.5.1. Bonding Descriptors

Bonding ‘descriptors’ on the drug and polymer molecules were used to identify potential sites for drug-drug and drug-polymer intermolecular binding interactions. The types of descriptors used include hydrogen bond donors (HBD), hydrogen bond acceptors (HBA), aromatic (AR), and hydrophobic (HPh).

To further elucidate potential sites for drug-drug and drug-polymer intermolecular binding interactions, surface area comparisons of the low energy conformations were performed. This method provides an overall estimation of the descriptor-based surface area available for intermolecular bonding (e.g. bonding between drug and polymer).

From the in silico modelling, Compound A was determined to have favorable interactions with HPMCAS, HPMC, PVP VA, HPMCP HP-55, PVP K30, and Eudragit L100-55. MDSC of Compound A provided a Tm/Tg ratio (K/K) of 1.30. The Tm/Tg ratio is a strong indicator of a molecule's crystal lattice energy and its propensity to recrystallize, providing an indicator of formulation design space where an SDI dispersion will be stable at a certain drug:polymer ratio. Based on historical Tm/Tg ratio experience and in silico molecular dynamics interactions, SDI formulations at 25 and 40% drug loading were nominated for feasibility SDI manufacturing.

### 3.6. Focused Screening of Solid Dispersion Polymers

### 3.6.1. Spray Dried Formulation Manufacturing

Thirteen prototype Compound A:polymer dispersion formulations (containing both free base and salt API forms) were chosen for feasibility screening. These formulations were spray dried from neat acetone, with the exception of HPMC-containing SDIs, which were sprayed from 89:11 Acetone:H2O. A summary of SDIs and recovery yields can be found in Table 16 below.

TABLE 16
Summary of Compound A Feasibility SDIs

| | | 25:70:5 | | | 25:70:5 | | | | | | | | |
|---|---|---|---|---|---|---|---|---|---|---|---|---|---|
| Form- | 25:75 | HPMCAS- | | | PVP- | | | | | | | | |
| ulation | HPMCAS- | L | 40:60 | 25:75 | VA: | 40:60 | 25:75 | 40:60 | | | 25:75 | | |
| (active: | L | Vit E | HPMCAS- | PVP- | Vit E | PVP- | HPMCAS- | HPMCAS- | 25:75 | 40:60 | HPMC | 25:75 | 25:75 |
| excipient) | | TPGS | L | VA | TPGS | VA | M | M | HPMCP | HPMCP | E3LV | PVP-VA | HPMC |
| Lot # | 5 | 6 | 7 | 8 | 9 | 10 | 11 | 12 | 13 | 14 | 15 | 16 | 17 |
| K9-983- | | | | | | | | | | | | | |
| Batch | 8.04 | 8.06 | 5.05 | 8.09 | 8.07 | 5.09 | 8.12 | 5.03 | 8.01 | 5.07 | 7.9 | 5.3 | 5.3 |
| Size, | | | | | | | | | | | | | |
| solids | | | | | | | | | | | | | |
| (g) | | | | | | | | | | | | | |
| API Lot | ES7718-611-P1 | | | | | | | | | | | ES7718-547-P1, | |
| Number | | | | | | | | | | | | ES7121-659-P1 | |
| API form | Free Base | | | | | | | | | | | Salt | |
| | | | | | | | | | | | Acetone: | | Acetone: |
| Spray | | | | | | | | | | | Water | | Water |
| Solvent | Acetone | | | | | | | | | | 89:11 | Acetone | 89:11 |
| Spray | 7 | 7 | 4.5 | 7 | 7 | 4.5 | 7 | 4.5 | 7 | 4.5 | 5 | 7 | 5 |
| Solution | | | | | | | | | | | | | |
| (wt. % | | | | | | | | | | | | | |
| total | | | | | | | | | | | | | |
| solids) | | | | | | | | | | | | | |
| Wet | 86.1 | 79.3 | 86.1 | 83.1 | 81.2 | 81.1 | 88.5 | 88.5 | 94.5 | 89.4 | 84.5 | 81.2 | 83.5 |
| SDI | | | | | | | | | | | | | |
| Yield | | | | | | | | | | | | | |
| (%) | | | | | | | | | | | | | |
| Dry SDI | 83.6 | 78.0 | 83.9 | 80.5 | 79.2 | 78.6 | 86.4 | 86.2 | 91.4 | 86.5 | 82.3 | 79.0 | 82.1 |
| Yield | | | | | | | | | | | | | |

### 3.6.2. Feasibility SDI Characterization

Initial SDI formulations were characterized by X-ray powder diffraction (XRPD), scanning electron microscopy (SEM), modulated differential scanning calorimetry (MSDC), headspace gas chromatography (GC-HS), and in vitro dissolution tests.

GC-HS was used to measure the residual acetone remaining from Compound A SDI material after secondary drying. The residual solvent in all formulations was below the acetone limit (5000 ppm) set forth by the International Conference on Harmonization (ICH). Table 17 shows the residual acetone results for the eight formulations.

TABLE 17
Summary of GC Headspace Results for Compound
A Feasibility SDIs after Secondary Drying.
(Limit of Quantitation (LOQ) = 200 ppm)

| Formulations | Residual Acetone (ppm) |
|---|---|
| 25:75 Compound A:HPMCAS-L SDI | Not detected |
| 25:70:5 Compound A:HPMCAS-L:TPGS | Not detected |
| SDI | |
| 40:60 Compound A:HPMCAS-L SDI | Not detected |
| 25:75 Compound A:PVP-VA SDI | <LOQ |
| 25:70:5 Compound A:PVP-VA:TPGS SDI | Not detected |
| 40:60 Compound A:PVP-VA SDI | <LOQ |
| 25:75 Compound A:HPMCAS-M SDI | Not detected |
| 40:60 Compound A:HPMCAS-M SDI | Not detected |
| 25:75 Compound A:HPMCP-HP55 SDI | <LOQ |
| 40:60 Compound A:HPMCP-HP55 SDI | <LOQ |
| 25:75 Compound A:HPMC SDI | <LOQ |
| 28.3:71.7 Compound A Salt:PVP-VA SDI | <LOQ |
| 28.3:71.7 Compound A Salt:HPMC SDI | <LOQ |

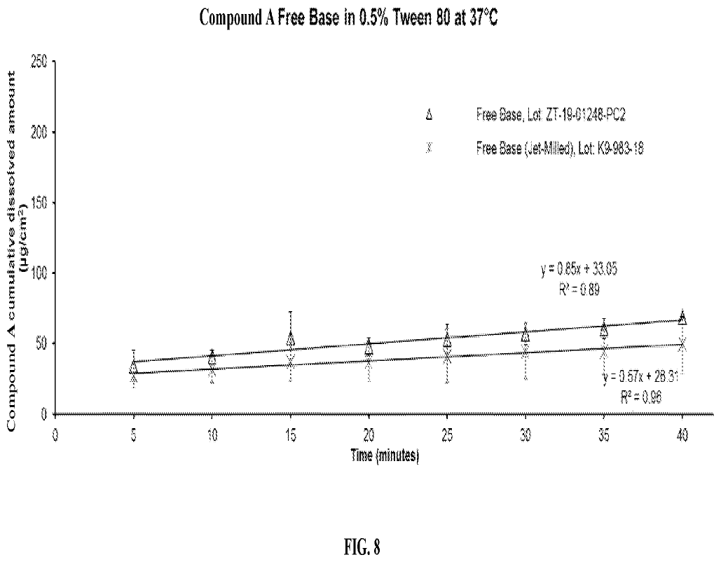

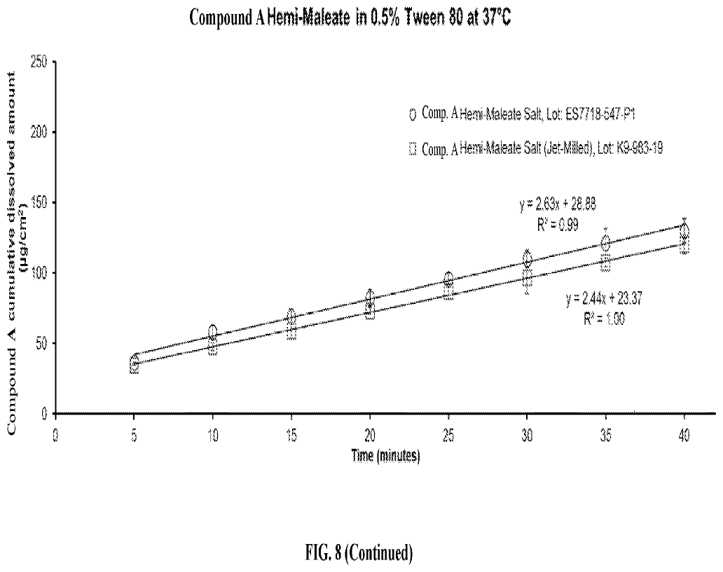

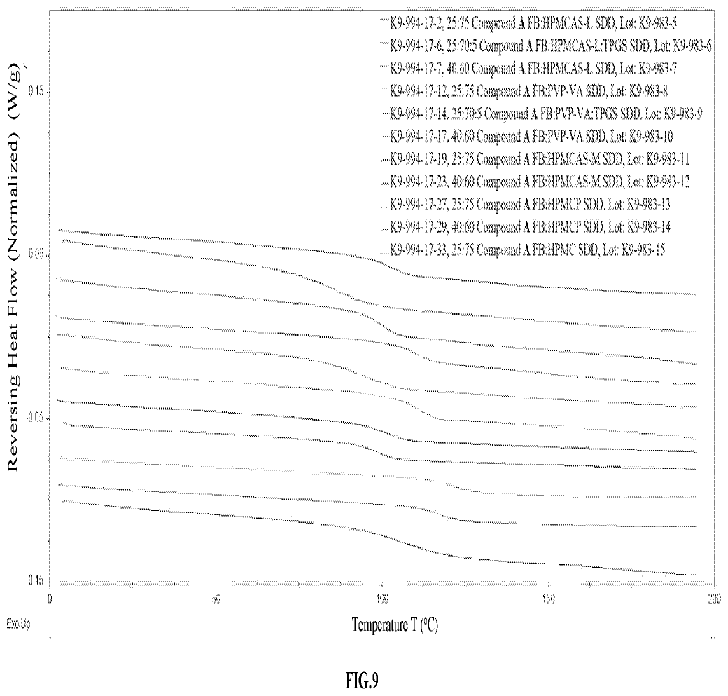

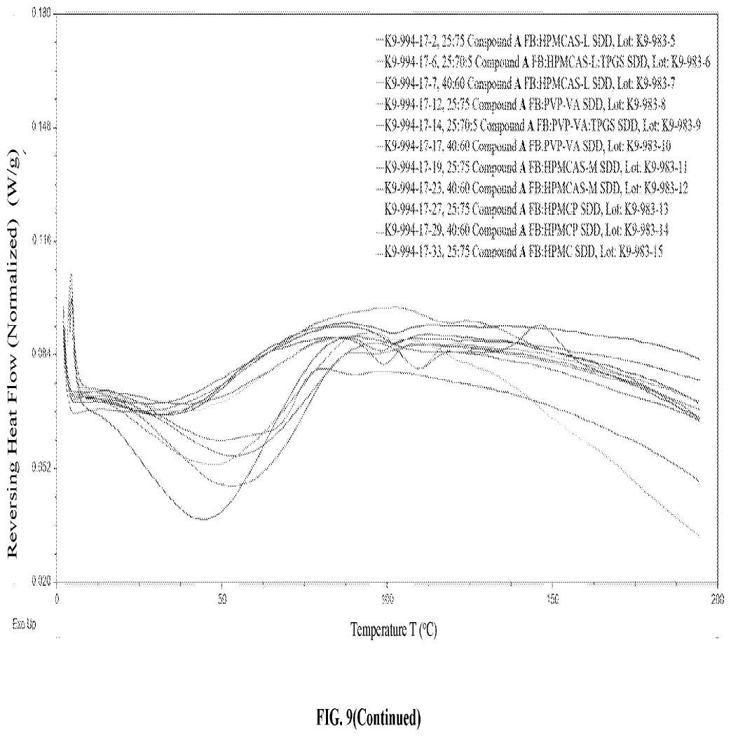

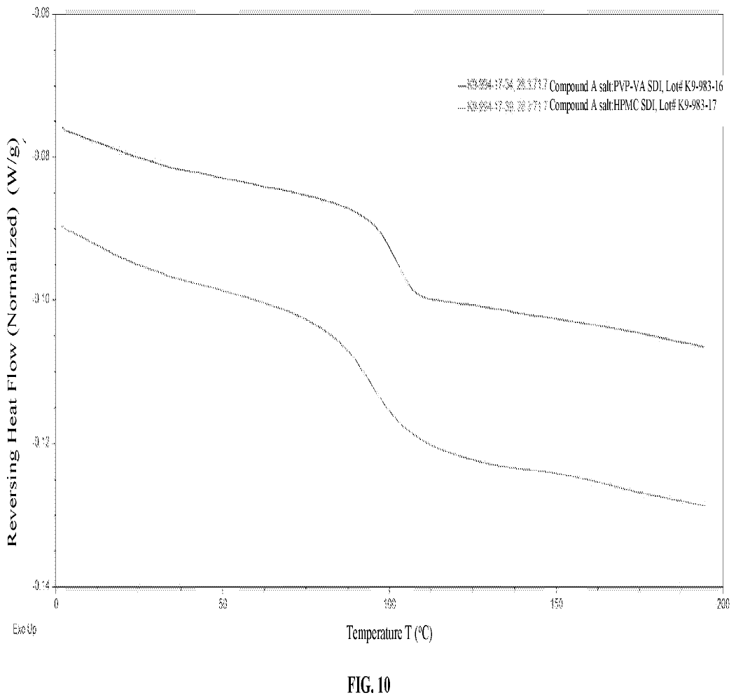

Thermal analysis by MDSC showed that all dispersions have a single Tg (FIGS. 9 and 10) indicating an intimately mixed amorphous solid dispersion with good homogeneity (Table 18) and no melting events were observed for any SDIs. These relatively high glass transition temperatures are an indication of good physical stability, i.e., the propensity of the API to recrystallize during long-term storage is low. To ensure long-term physical stability, an SDI should be stored well below the Tg at a given condition so that mobility of the drug in the glass dispersion is very low.

TABLE 18
MDSC Data for Compound A Feasibility SDIs.

| Formulations | Measured Tg (° C.) |
|---|---|
| 25:75 Compound A:HPMCAS-L SDI | 102 |
| 25:70:5 Compound A:HPMCAS-L:TPGS SDI | 87 |
| 40:60 Compound A:HPMCAS-L SDI | 100 |
| 25:75 Compound A:PVP-VA SDI | 110 |
| 25:70:5 Compound A:PVP-VA:TPGS SDI | 94 |
| 40:60 Compound A:PVP-VA SDI | 110 |
| 25:75 Compound A:HPMCAS-M SDI | 101 |
| 40:60 Compound A:HPMCAS-M SDI | 99 |
| 25:75 Compound A:HPMCP-HP55 SDI | 122 |
| 40:60 Compound A:HPMCP-HP55 SDI | 118 |
| 25:75 Compound A:HPMC SDI | 106 |
| 28.3:71.7 Compound A Salt:PVP-VA SDI | 101 |
| 28.3:71.7 Compound A Salt:HPMC SDI | 95 |

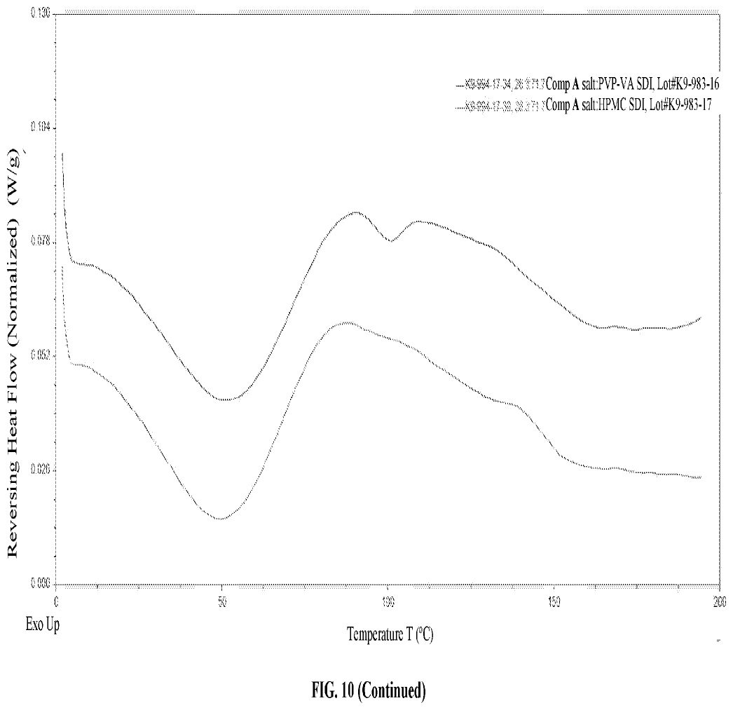

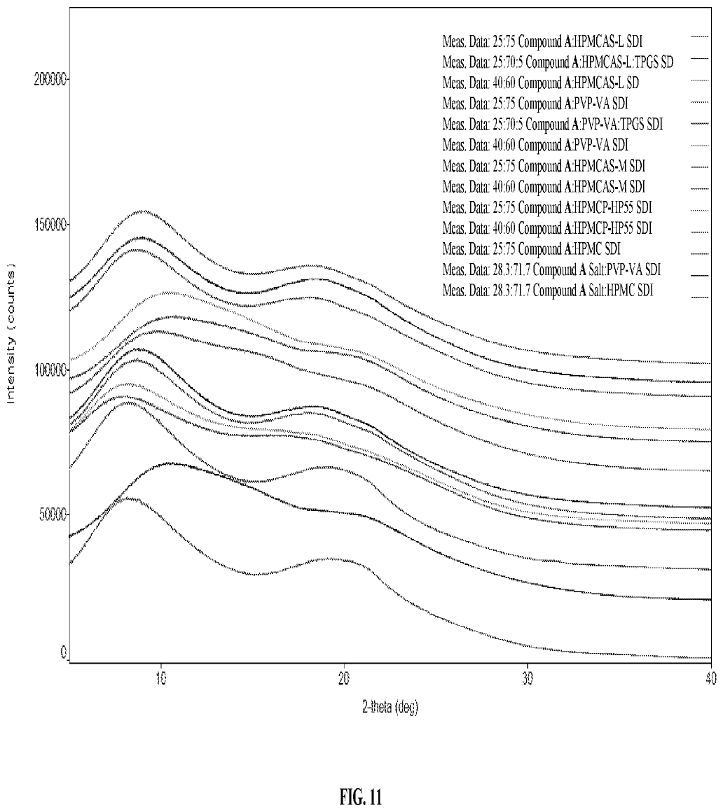

Initial characterization by XRPD indicates that the SDIs are amorphous dispersions and no crystalline peaks were observed in the SDI diffractograms (FIG. 11).

Surface morphology of the SDI particles was characterized using scanning electron microscopy (The SEM images are not provided). Typical SDI morphology was observed consisting of whole and collapsed spheres with smooth surfaces. No crystalline material was observed in any samples.

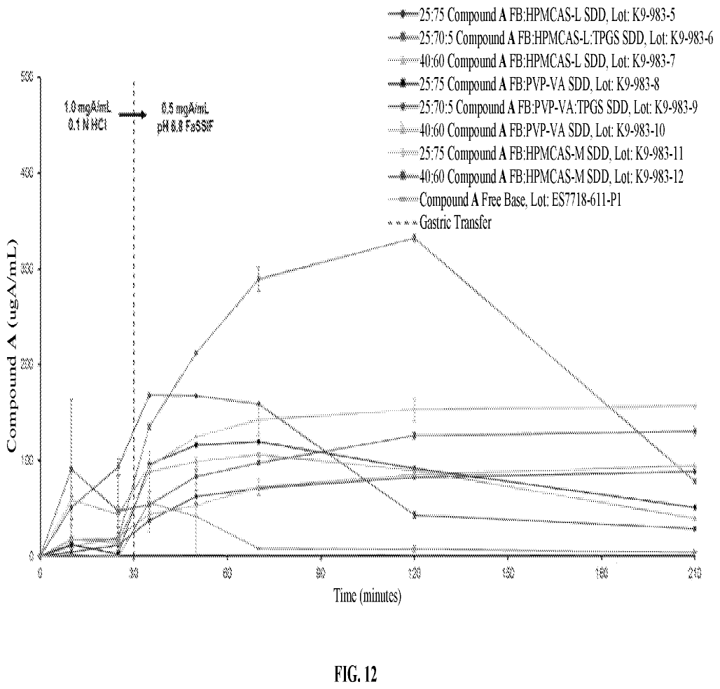

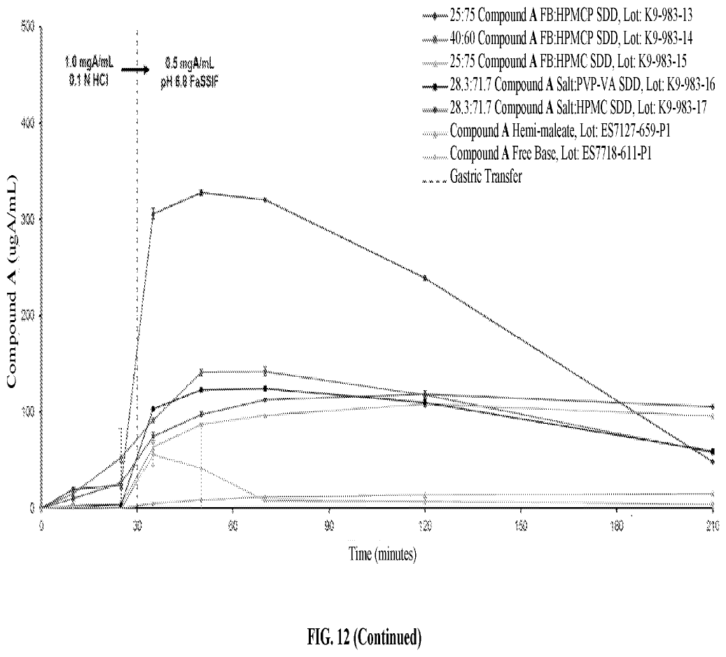

The dissolution performance of the feasibility SDIs and crystalline Compound A was tested in the non-sink dissolution test (FIG. 12 and Table 19). The design of this experiment is to rank order and select lead formulations. All SDI formulations provided an increase in drug dissolution and sustainment in intestinal media, with 25:70:5 Compound A:HPMCAS-L:TPGS showing the highest increase in supersaturation, but did crash out at the final time point. Gastric solubility was low among the SDIs, with no observed correlation between gastric dissolution levels and subsequent intestinal dissolution levels. The two maleate salt SDIs did outperform the bulk maleate salt but were considerably lower performers and were not selected for advancement.

TABLE 19
Non-Sink Dissolution Data for Compound A Feasibility
SDIs Compared to Bulk Crystalline Compound A.

| | | | ³AUC₃₅₋₂₁₀ |
|---|---|---|---|
| | ¹CₘₐₓFaSSIF | ²C₂₁₀ | FaSSIF |
| Formulations | (μgA/mL) | (μgA/mL) | (min*μgA/mL) |
| 25:75 Compound A:HPMCAS-L SDI | 88.3 | 88.3 | 13600 |
| 25:70:5 Compound A:HPMCAS-L:TPGS SDI | 332.2 | 78.3 | 41600 |
| 40:60 Compound A:HPMCAS-L SDI | 94.7 | 94.7 | 14000 |
| 25:75 Compound A:PVP-VA SDI | 119.5 | 51.0 | 15600 |
| 25:70:5 Compound A:PVP-VA:TPGS SDI | 168.0 | 28.5 | 14000 |
| 40:60 Compound A:PVP-VA SDI | 106.3 | 39.1 | 14100 |
| 25:75 Compound A:HPMCAS-M SDI | 156.7 | 156.7 | 25600 |
| 40:60 Compound A:HPMCAS-M SDI | 130.3 | 130.3 | 20000 |
| 25:75 Compound A:HPMCP-HP55 SDI | 328.0 | 47.9 | 38100 |
| 40:60 Compound A:HPMCP-HP55 SDI | 142.1 | 57.4 | 18900 |
| 25:75 Compound A:HPMC SDI | 108.0 | 95.8 | 17200 |
| 28.3:71.7 Compound A Salt:PVP-VA SDI | 124.0 | 59.4 | 17600 |
| 28.3:71.7 Compound A Salt:HPMC SDI | 118.5 | 105.5 | 19300 |
| Compound A Free Base | 55.6 | 4.5 | 2100 |
| Compound A Maleate Salt | 15.4 | 15.4 | 2300 |

¹Cₘₐₓ FaSSIF = maximum drug concentration after transfer to FaSSIF
²C₂₁₀ = drug concentration at 180 minutes after transfer to FaSSIF
³AUC₃₅₋210 FaSSIF = area under the curve after transfer to FaSSIF from 35 to 210 minutes

Lead SDIs were selected mostly on dissolution performance, while also keeping physical properties in mind. Five SDIs were selected and will be placed on accelerated stability and undergo assay, impurities analysis by HPLC, and Tg as a function of % relative humidity. Lead formulations are 25:70:5 Compound A:HPMCAS-L:TPGS, 40:60 Compound A:PVP-VA, 40:60 Compound A:HPMCAS-M, and 25% and 40% Compound A:HPMCP-HP55.

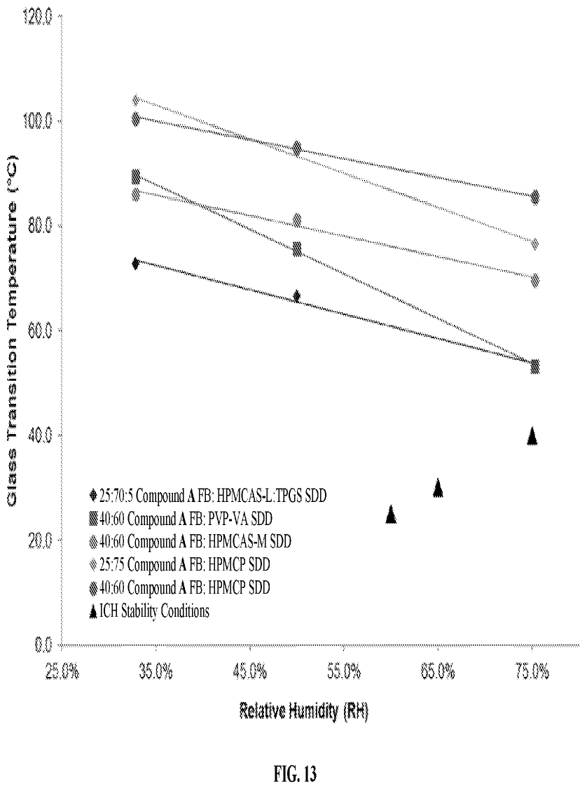

The suppression of the Tg of the lead SDIs were evaluated by measuring the Tg at elevated humidity (32.8%, 50%, and 75.3% RH) conditions. Samples were stored at the elevated humidity conditions for 18 hours at ambient temperature before sealing in hermetic pans and analysis by MDSC. Results are reported as a function of relative humidity (RH) in FIG. 13 and Table 20. All lead SDI formulations (HPMCAS-H and PVP VA64 dispersions) have a Tg that is low at elevated humidity conditions and is predicted to require conservative packaging (i.e., desiccant, foil-foil seal, or etc.) to obtain sufficient long-term physical stability of the SDI. To ensure long-term physical stability in open packaging at all ICH conditions, it is desirable that the SDI have a Tg higher than 50° C. at 75% RH, and ideally higher than 60° C. at 75% RH.

TABLE 20
Tg as a Function of % RH for Compound
A Lead SDI Formulations.

| | Tg at RH (° C.) | | |
|---|---|---|---|
| | 32.8% | 50.0% | 75.3% |
| Sample | RH | RH | RH |
| 25:70:5 Compound A FB:HPMCAS-L:TPGS | 73 | 67 | 53 |
| SDI | | | |
| 40:60 Compound A FB:PVP-VA SDI | 89 | 76 | 53 |
| 40:60 Compound A FB:HPMCAS-M SDI | 86 | 81 | 70 |
| 25:75 Compound A FB:HPMCP SDI | 104 | 94 | 76 |
| 40:60 Compound A FB:HPMCP SDI | 101 | 95 | 85 |

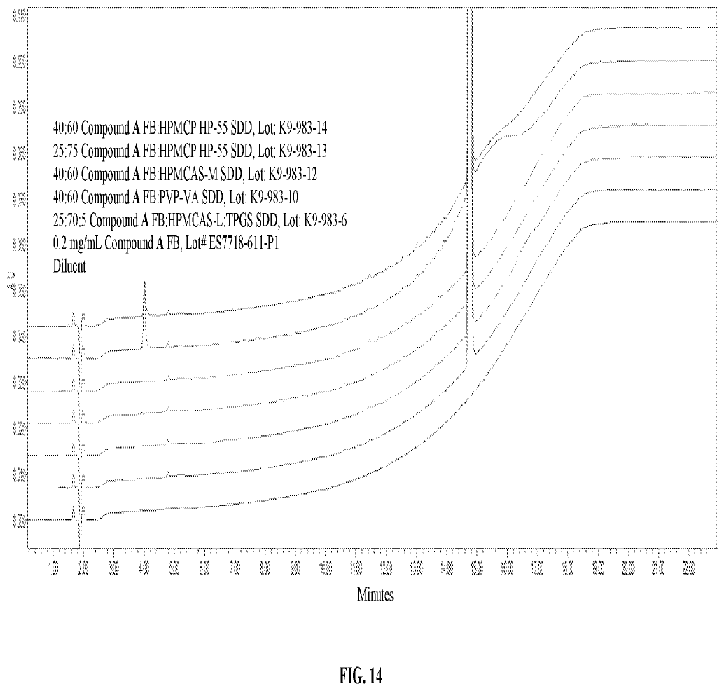

Assay and impurities analysis of the lead SDIs by HPLC was performed at t=0 utilizing the HPMC method described in section 2.2. All SDIs show similar impurity profiles compared to bulk API, indicating no minimal degradation occurred during the spray drying process. Both HPMCP SDIs showed a large early eluting peak at RRT 0.27 which is attributed to phthalic acid, Table 21 and FIG. 14.

TABLE 21
t = 0 Assay, Impurities Data for Compound A Lead SDI Formulations

| | | 25:70:5 Comp A | | | 25:75 Comp A | 40:60 Comp A |
|---|---|---|---|---|---|---|
| | Comp A | FB: HPMCAS- | 40:60 Comp A | 40:60 Comp A | FB:HPMCP | FB:HPMCP |
| Material | FreeBase | L:TPGS | FB:PVP-VA | FB:HPMCAS-M | HP55 | HP55 |
| RRT | | | | | | |
| 0.27 | | | | | 1.17% | 0.73% |
| 0.32 | 0.05% | 0.05% | 0.05% | 0.06% | 0.06% | 0.05% |
| 0.49 | | | | | Detected, <0.05% | |
| 0.52 | | | | | Detected, <0.05% | |
| 0.74 | | | | | | Detected, <0.05% |
| 0.78 | | Detected, <0.05% | Detected, <0.05% | 0.09% | Detected, 0.05% | Detected, 0.05% |
| 0.80 | | | Detected, <0.05% | Detected, <0.05% | | |
| 0.82 | | | | Detected, <0.05% | | Detected, <0.05% |
| 0.85 | | 0.05% | | Detected, <0.05% | 0.08% | Detected, <0.05% |
| 0.88 | | Detected, <0.05% | Detected, <0.05% | 0.07% | 0.05% | 0.05% |
| 0.90 | | | | | Detected, <0.05% | Detected, <0.05% |
| 0.92 | Detected, | | | Detected, <0.05% | Detected, <0.05% | Detected, <0.05% |
| | <0.05% | | | | | |
| 0.94 | | Detected, <0.05% | | Detected, <0.05% | | Detected, <0.05% |
| Total | 0.05% | 0.11% | 0.05% | 0.22% | 1.36% | 0.84% |
| Assay | 100.0 | 25.2 ± 0.1 | 39.5 ± 0.1 | 38.7 ± 0.0 | 24.6 ± 0.2 | 39.7 ± 0.0 |
| (wt %) | | | | | | |

### 3.6.3. Feasibility SDS Accelerated Stability

To rapidly assess the physical and chemical stability of the Compound A lead SDI formulations, the dispersions were aged for 4 weeks at 25° C./60% RH in open packaging, and 40° C./75% RH in open and closed packaging per stability protocol RD-ST-19-919. The SDIs were evaluated for physical and chemical stability by appearance, amorphous character by XRPD, and assay and impurities by HPLC. Based on the 4-week stability data 40:60 Compound A:HPMCAS-M was nominated for progression into tablet development and GMP activities, and was analyzed at a second time point of 10 weeks at 40° C./75% RH open and closed only.

Appearance testing of the aged SDIs revealed that all remained an off white powder throughout the stability study, even at elevated humidity and temperature, Table 22 below.

TABLE 22
Visual Appearance of Compound A Lead SDIs after 4-10 Weeks Stability

| Sample | Storage condition | Appearance |
|---|---|---|
| 25:70:5 Compound A | T = 0 | Off-White Powder |
| FB:HPMCAS-L:TPGS SDI | 4 week/25° C./60% RH/OPEN | Off-White Powder |
| | 4 week/40° C./75% RH/OPEN | Off-White Powder |
| | 4 week/40° C./75% RH/CLOSED | Off-White Powder |
| 40:60 Compound A FB:PVP-VA | T = 0 | Off-White Powder |
| SDI | 4 week/40° C./75% RH/OPEN | Off-White Powder |
| | 4 week/40° C./75% RH/CLOSED | Off-White Powder |
| 40:60 Compound A FB:HPMCAS- | T = 0 | Off-White Powder |
| M SDI | 4 week/40° C./75% RH/OPEN | Off-White Powder |
| | 4 week/40° C./75% RH/CLOSED | Off-White Powder |
| | 10 week/40° C./75% RH/OPEN | Off-White Powder |
| | 10 week/40° C./75% RH/CLOSED | Off-White Powder |
| 25:75 Compound A FB:HPMCP- | T = 0 | Off-White Powder |
| HP55 SDI | 4 week/25° C./60% RH/OPEN | Off-White Powder |
| | 4 week/40° C./75% RH/OPEN | Off-White Powder |
| | 4 week/40° C./75% RH/CLOSED | Off-White Powder |
| 40:60 Compound A FB:HPMCP- | T = 0 | Off-White Powder |
| HP55 SDI | 4 week/40° C./75% RH/OPEN | Off-White Powder |
| | 4 week/40° C./75% RH/CLOSED | Off-White Powder |

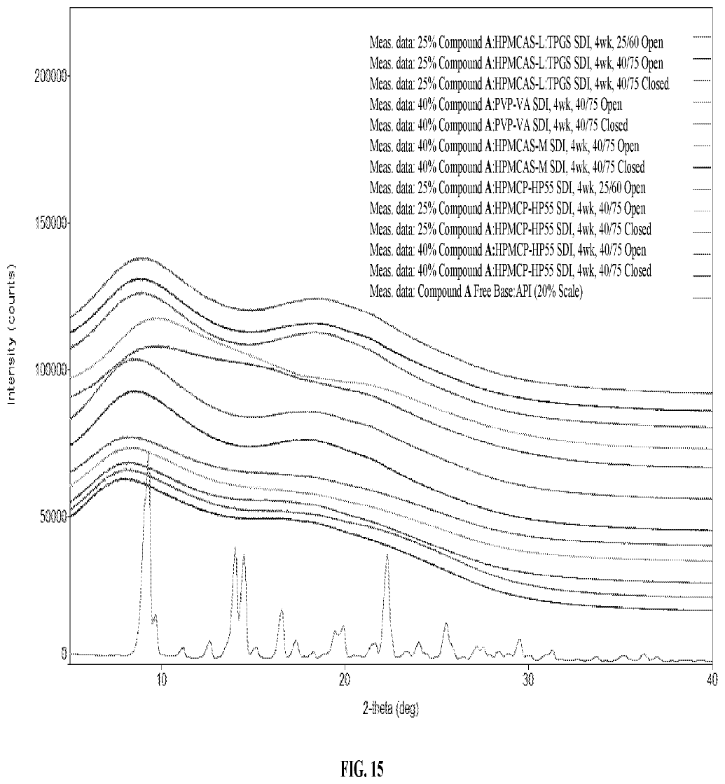

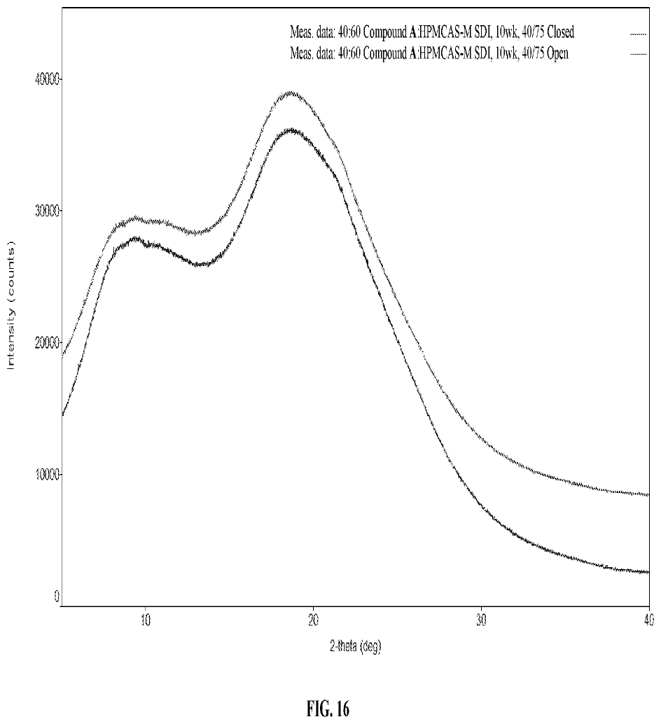

XRPD analysis of the aged SDI samples shows that all SDI formulations remain amorphous with no detectable crystalline material after 4 weeks, FIG. 15. After 10 weeks 40:60 Compound A:HPMCAS-M remained an amorphous dispersion as well, FIG. 16.

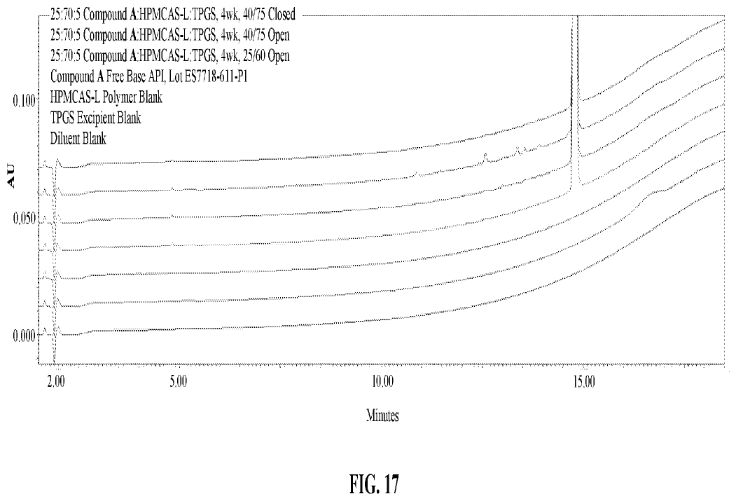

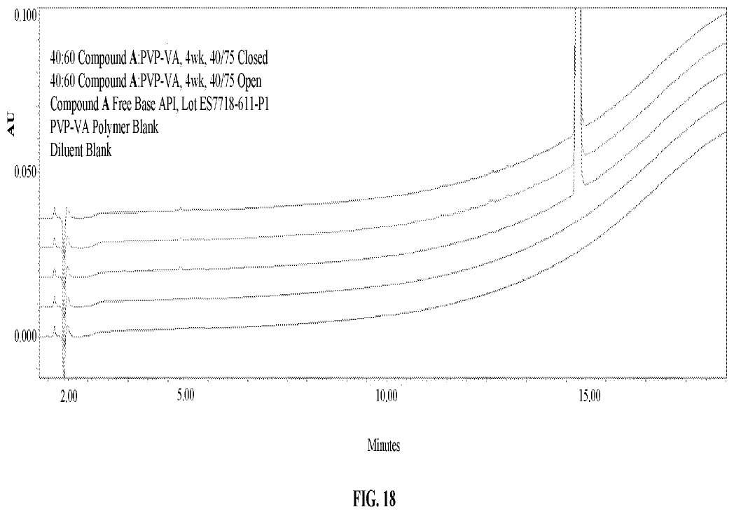

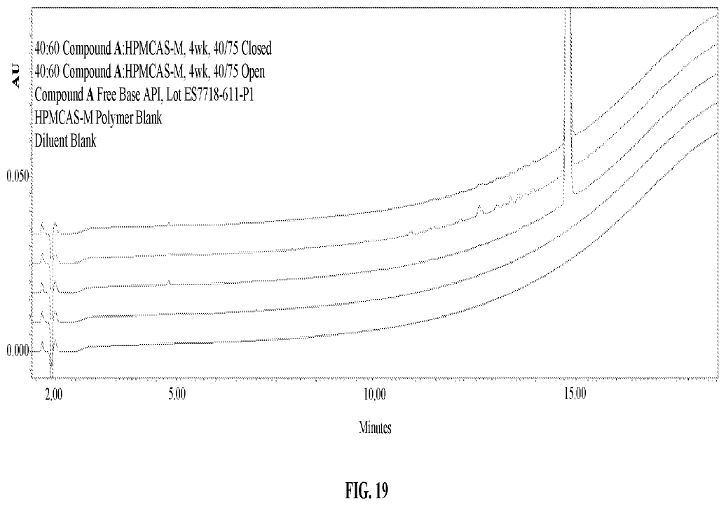

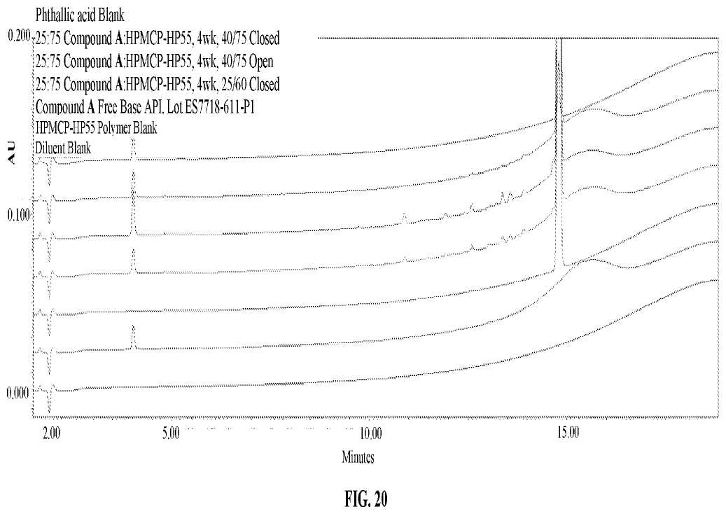

Assay and impurities analysis of the aged SDI samples showed minimal growth in the closed conditions, however in open condition there was significant degradation observed, indicating moisture protective packaging will likely be required (FIGS. 17-21 and Tables 23-27).

TABLE 23
Assay, Impurities Data for 25:70:5 Compound A:HPMCAS-L:TPGS
SDI Compared to Bulk Crystalline API After 4 Weeks Stability.

| Material | Compound A FB | 25:70:5 Compound A FB HPMCAS-L:TPGS SDI | | | |
|---|---|---|---|---|---|
| Condition | — | t = 0 | 4 wk 25° C./60% RH | 4 wk 40° C./75% RH | 4 wk 40° C./75% RH |
| | | | (OPEN) | (OPEN) | (CLOSED) |
| RRT | | | | | |
| 0.33 | 0.05% | 0.05% | 0.05% | 0.05% | 0.07% |
| 0.66 | | | | Detected, <0.05% | |
| 0.74 | | | | 0.13% | |
| 0.77 | | Detected, <0.05% | | Detected, <0.05% | |
| 0.77 | | | | 0.06% | |
| 0.82 | | | Detected, <0.05% | | |
| 0.84 | | | | Detected, <0.05% | |
| 0.85 | Detected, <0.05% | 0.05% | 0.07% | 0.33% | Detected, <0.05% |
| 0.86 | | | Detected, <0.05% | | |
| 0.88 | | Detected, <0.05% | Detected, <0.05% | 0.06% | |
| 0.89 | | | | Detected, <0.05% | |
| 0.90 | | | Detected, <0.05% | 0.24% | |
| 0.92 | | | Detected, <0.05% | 0.12% | |
| 0.93 | Detected, <0.05% | | Detected, <0.05% | Detected, <0.05% | Detected, <0.05% |
| 0.94 | | Detected, <0.05% | Detected, <0.05% | 0.11% | Detected, <0.05% |
| 0.99 | | | | 0.11% | |
| Total | 0.05% | 0.11% | 0.12% | 1.21% | 0.07% |
| Assay | 100.0 | 25.2 ± 0.1 | 24.8 ± 0.0 | 24.4 ± 0.3 | 25.4 ± 0.1 |
| (wt %) | | | | | |

TABLE 24
Assay, Impurities Data for 40:60 Compound A:PVP-VA SDI Compared
to Bulk Crystalline API After 4 Weeks Stability.

| | Material | | | |
|---|---|---|---|---|
| | Compound A FB | 40:60 Compound A FB:PVP-VA SDI | | |
| | Condition | | | |
| | | | 4 wk 40° | 4 wk 40° |
| | | | C./75% RH | C./75% RH |
| RRT | — | t = 0 | (OPEN) | (CLOSED) |
| 0.33 | 0.05% | 0.05% | 0.05% | 0.06% |
| 0.77 | | | 0.05% | |
| 0.77 | | | Detected, | |
| | | | <0.05% | |
| 0.78 | | Detected, | | |
| | | <0.05% | | |
| 0.80 | | Detected, | | |
| | | <0.05% | | |
| 0.82 | | | Detected, | |
| | | | <0.05% | |
| 0.85 | Detected, | | 0.07% | Detected, |
| | <0.05% | | | <0.05% |
| 0.86 | | | 0.05% | Detected, |
| | | | | <0.05% |
| 0.88 | | Detected, | 0.05% | Detected, |
| | | <0.05% | | <0.05% |
| 0.92 | | | Detected, | |
| | | | <0.05% | |
| 0.93 | Detected, | | 0.05% | Detected, |
| | <0.05% | | | <0.05% |
| 0.94 | | | Detected, | |
| | | | <0.05% | |
| Total | 0.05% | 0.05% | 0.33% | 0.06% |
| Assay | 100.0 | 39.5 ± 0.1 | 38.5 ± 0.1 | 39.6 ± 0.3 |
| (wt %) | | | | |

TABLE 25
Assay, Impurities Data for 40:60 Compound A:HPMCAS-M Compared
to Bulk Crystalline API After 4 Weeks Stability.

| | Material | | | |
|---|---|---|---|---|
| | Compound A FB | 40:60 Compound A FB:HPMCAS-M SDI | | |
| | Condition | | | |
| | | | 4 wk 40° | 4 wk 40° |
| | | | C./75% RH | C./75% RH |
| RRT | — | t = 0 | (OPEN) | (CLOSED) |
| 0.33 | 0.05% | 0.06% | 0.05% | 0.05% |
| 0.66 | | | Detected, | |
| | | | <0.05% | |
| 0.74 | | | 0.08% | |
| 0.77 | | | 0.05% | |
| 0.77 | | 0.09% | 0.06% | Detected, |
| | | | | <0.05% |
| 0.80 | | Detected, | | |
| | | <0.05% | | |
| 0.82 | | Detected, | 0.06% | Detected, |
| | | <0.05% | | <0.05% |
| 0.85 | Detected, | Detected, | 0.21% | 0.06% |
| | <0.05% | <0.05% | | |
| 0.86 | | | 0.07% | Detected, |
| | | | | <0.05% |
| 0.88 | | 0.07% | 0.10% | Detected, |
| | | | | <0.05% |
| 0.89 | | | 0.05% | |
| 0.90 | | | 0.14% | Detected, |
| | | | | <0.05% |
| 0.92 | | Detected, | 0.13% | Detected, |
| | | <0.05% | | <0.05% |
| 0.93 | Detected, | | Detected, | 0.05% |
| | <0.05% | | <0.05% | |
| 0.93 | | | Detected, | |
| | | | <0.05% | |
| 0.94 | | Detected, | 0.11% | 0.05% |
| | | <0.05% | | |
| 0.99 | | | 0.06% | |
| Total | 0.05% | 0.22% | 1.17% | 0.21% |
| Assay | 100.0 | 38.7 ± 0.0 | 37.7 ± 0.5 | 38.2 ± 1.1 |
| (wt %) | | | | |

TABLE 26
Assay, Impurities Data for 25:75 Compound A:HPMCP-HP55 Compared
to Bulk Crystalline API After 4 Weeks Stability.

| | Material | | | | |
|---|---|---|---|---|---|
| | Compound A FB | 25:75 Compound A FB HPMCP SDI | | | |
| | Condition | | | | |
| | | | 4 wk 25° | 4 wk 40° | 4 wk 40° |
| | | | C./60% RH | C./75% RH | C./75% RH |
| RRT | — | t = 0 | (OPEN) | (OPEN) | (CLOSED) |
| 0.27 | | 1.17% | 1.01% | 1.88% | 1.09% |
| (Phthalic | | | | | |
| acid) | | | | | |
| 0.33 | 0.05% | 0.06% | 0.05% | 0.05% | 0.05% |
| 0.50 | | Detected, | Detected, | Detected, | Detected, |
| | | <0.05% | <0.05% | <0.05% | <0.05% |
| 0.52 | | Detected, | | | |
| | | <0.05% | | | |
| 0.53 | | | | Detected, | Detected, |
| | | | | <0.05% | <0.05% |
| 0.53 | | | Detected, | Detected, | |
| | | | <0.05% | <0.05% | |
| 0.63 | | | | Detected, | |
| | | | | <0.05% | |
| 0.66 | | | Detected, | 0.08% | |
| | | | <0.05% | | |
| 0.68 | | | Detected, | Detected, | |
| | | | <0.05% | <0.05% | |
| 0.73 | | | | Detected, | |
| | | | | <0.05% | |
| 0.74 | | | 0.27% | 0.53% | Detected, |
| | | | | | <0.05% |
| 0.76 | | | | 0.10% | |
| 0.77 | | | 0.05% | 0.08% | Detected, |
| | | | | | <0.05% |
| 0.77-0.78 | | Detected, | 0.07% | 0.05% | |
| | | <0.05% | | | |
| 0.81 | | | 0.07% | 0.23% | |
| 0.81 | | | Detected, | | |
| | | | <0.05% | | |
| 0.82 | | | 0.05% | Detected, | |
| | | | | <0.05% | |
| 0.84 | | | Detected, | 0.07% | |
| | | | <0.05% | | |
| 0.85 | Detected, | 0.08% | 0.28% | 0.35% | 0.07% |
| | <0.05% | | | | |
| 0.88 | | 0.05% | 0.16% | Detected, | Detected, |
| | | | | <0.05% | <0.05% |
| 0.89 | | | 0.06% | 0.07% | |
| 0.89 | | | | 0.12% | |
| 0.90 | | Detected, | 0.23% | 0.47% | Detected, |
| | | <0.05% | | | <0.05% |
| 0.92 | | Detected, | 0.22% | 0.41% | Detected, |
| | | <0.05% | | | <0.05% |
| 0.93 | Detected, | | 0.11% | | Detected, |
| | <0.05% | | | | <0.05% |
| 0.94 | | | 0.19% | 0.28% | 0.09% |
| 0.99 | | | 0.12% | 0.45% | |
| Total | 0.05% | 1.36% | 2.95% | 5.22% | 1.31% |
| Assay | 100.0 | 24.6 ± 0.2 | 23.5 ± 0.1 | 23.0 ± 0.1 | 24.7 ± 0.1 |
| (wt %) | | | | | |

TABLE 27
Assay, Impurities Data for 40:60 Compound A:HPMCP-HP55 SDI
Compared to Bulk Crystalline API After 4 Weeks Stability.

| | Material | | | |
|---|---|---|---|---|
| | Compound A FB | 40:60 Compound A FB:HPMCP SDI | | |
| | Condition | | | |
| | | | 4 wk 40° | 4 wk 40° |
| | | | C./75% RH | C./75% RH |
| RRT | — | t = 0 | (OPEN) | (CLOSED) |
| 0.27 | | 0.73% | 0.95% | 0.62% |
| (Phthalic | | | | |
| acid) | | | | |
| 0.33 | 0.05% | 0.05% | 0.06% | 0.05% |
| 0.50 | | | | Detected, |
| | | | | <0.05% |
| 0.66 | | | 0.05% | |
| 0.74 | | Detected, | 0.35% | Detected, |
| | | <0.05% | | <0.05% |
| 0.76 | | | 0.06% | |
| 0.77 | | Detected, | 0.08% | Detected, |
| | | <0.05% | | <0.05% |
| 0.81 | | | 0.11% | |
| 0.81 | | | Detected, | |
| | | | <0.05% | |
| 0.82 | | Detected, | Detected, | Detected, |
| | | <0.05% | <0.05% | <0.05% |
| 0.84 | | | Detected, | |
| | | | <0.05% | |
| 0.85 | Detected, | Detected, | 0.28% | 0.08% |
| | <0.05% | <0.05% | | |
| 0.86 | | | | Detected, |
| | | | | <0.05% |
| 0.88 | | 0.05% | 0.06% | Detected, |
| | | | | <0.05% |
| 0.88 | | | Detected, | |
| | | | <0.05% | |
| 0.89 | | | 0.08% | |
| 0.90 | | Detected, | 0.32% | Detected, |
| | | <0.05% | | <0.05% |
| 0.92 | | | 0.26% | 0.05% |
| 0.93 | Detected, | Detected, | Detected, | Detected, |
| | <0.05% | <0.05% | <0.05% | <0.05% |
| 0.93 | | | Detected, | |
| | | | <0.05% | |
| 0.94 | | Detected, | 0.15% | 0.08% |
| | | <0.05% | | |
| 0.99 | | | 0.27% | |
| Total | 0.05% | 0.84% | 3.07% | 0.87% |
| Assay | 100.0 | 39.7 ± 0.0 | 37.8 ± 0.5 | 39.5 ± 0.2 |
| (wt %) | | | | |

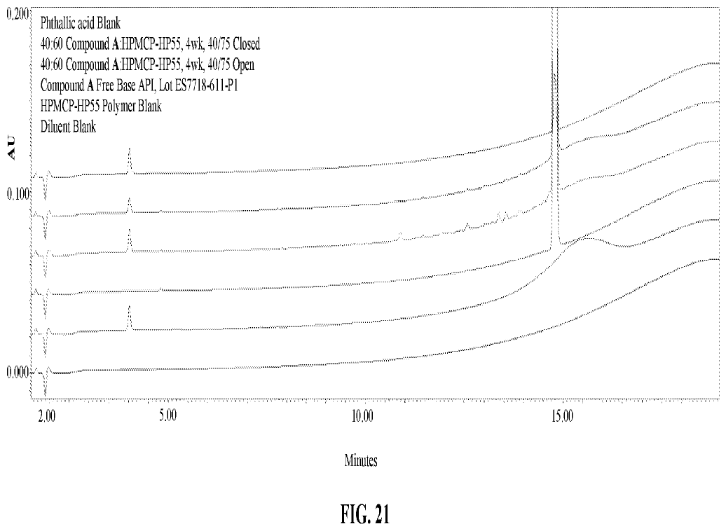

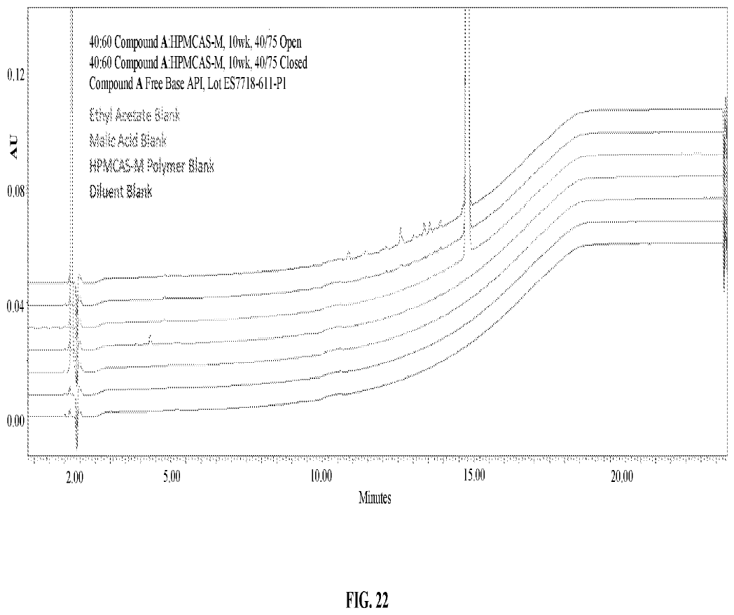

As previously mentioned, 40:60 Compound A:HPMCAS-M was characterized after 10 weeks stability by assay, impurities. Open condition samples show considerable impurity growth, most notably at RRT 0.85. Closed condition samples show no significant increase in impurities, indicating a moisture mediated degradation pathway, with thermal degradation less of a concern, Table 28 and FIG. 22.

TABLE 28
Assay, Impurities Data for 40:60 Compound A:HPMCAS-M SDI Compared to
Bulk Crystalline API After 10 Weeks Stability.

| | Compound A | | | | | |
|---|---|---|---|---|---|---|
| Material | FB | 40:60 Compound A FB:HPMCAS-M SDI | | | | |
| | | | 4 | 10 | 4 | 10 |
| | | | weeks/40^{o} C./75% | weeks/40^{o} C./75% | weeks/40^{o} C./75% | weeks/40^{o} C./75% |
| Condition | — | t = 0 | RH (OPEN) | RH (OPEN) | RH (CLOSED) | RH (CLOSED) |
| RRT | | | | | | |
| 0.33 | 0.05% | 0.06% | 0.05% | Detected, <0.05% | 0.05% | 0.06% |
| 0.63 | | | | Detected, <0.05% | | |
| 0.63 | | | | Detected, <0.05% | | |
| 0.66 | | | Detected, <0.05% | 0.06% | | |
| 0.74 | | | 0.08% | 0.22% | | Detected, <0.05% |
| 0.77 | | | 0.05% | 0.08% | | Detected, <0.05% |
| 0.77 | | 0.09% | 0.06% | 0.12% | Detected, <0.05% | Detected, <0.05% |
| 0.80 | | Detected, <0.05% | | Detected, <0.05% | | |
| 0.81 | | | | Detected, <0.05% | | |
| 0.82 | | Detected, <0.05% | 0.06% | 0.09% | Detected, <0.05% | 0.05% |
| 0.84 | | | | Detected, <0.05% | | |
| 0.85 | Detected, <0.05% | Detected, <0.05% | 0.21% | 0.51% | 0.06% | 0.09% |
| 0.86 | | | 0.07% | | Detected, <0.05% | Detected, <0.05% |
| 0.88 | | 0.07% | 0.10% | 0.16% | Detected, <0.05% | 0.05% |
| 0.89 | | | 0.05% | 0.10% | | |
| 0.90 | | | 0.14% | 0.32% | Detected, <0.05% | Detected, <0.05% |
| 0.92 | | Detected, <0.05% | 0.13% | 0.27% | Detected, <0.05% | 0.05% |
| 0.93 | Detected, <0.05% | | Detected, <0.05% | 0.10% | 0.05% | 0.05% |
| 0.93 | | | Detected, <0.05% | | | |
| 0.94 | | Detected, <0.05% | 0.11% | 0.19% | 0.05% | 0.08% |
| 0.99 | | | 0.06% | 0.19% | | |
| Total | 0.05% | 0.22% | 1.17% | 2.41% | 0.21% | 0.45% |
| Assay | 100.0 | 38.7 ± 0.0 | 37.7 ± 0.5 | 37.2 ± 0.1 | 38.2 ± 1.1 | 38.9 ± 0.1 |
| (wt %) | | | | | | |

### 3.7. SDI Demonstration Batch

### 3.7.1. SDI Demonstration Batch Manufacture

Based on PK performance of Compound A SDI suspensions prepared from the feasibility SDIs along with the in vitro dissolution performance and the 4-week stability pull data, 40:60 Compound A FB:HPMCAS-M was nominated as the lead SDI formulation and progressed for the development of the tablet formulations. A demonstration batch was manufactured according to GLPs on a pilot scale.

### 3.7.2. SDI Demonstration Batch Characterization

The dry demonstration SDI formulation was characterized by X-ray powder diffraction (XRPD), scanning electron microscopy (SEM), modulated differential scanning calorimetry (MSDC), headspace gas chromatography (GC-HS), and assay, impurities by HPLC. Assay, impurities by HPLC was also performed on the spray solution and wet SDI to observe any potential chemical degradation occurring during excessive solution or wet SDI hold times.

GC-HS analysis was used to measure the residual acetone remaining from Compound A SDI material after secondary drying, with samples taken prior to secondary drying, “wet SDI”, and at additional time points to create a drying curve showing the removal of acetone from the SDI over time. The residual solvent was below the ICH limit after only 2 hours, and was not detected after 18.5 hours. Table 29 shows the residual acetone results for the demonstration batch SDI.

TABLE 29
Summary of GC-HS Drying Curve Data for Compound
A Demonstration SDI after Secondary Drying

| | Residual |
|---|---|
| Sample Description | Acetone (ppm) |
| 40:60 Compound A FB:HPMCAS-M | 28,000 |
| Wet SDI, t = 0 | |
| 40:60 Compound A FB:HPMCAS-M | 3,200 |
| Wet SDI, t = 2 hr | |
| 40:60 Compound A FB:HPMCAS-M | 430 |
| Wet SDI, t = 9 hr | |
| 40:60 Compound A FB:HPMCAS-M | <200 |
| Wet SDI, t = 18.5 hr | |
| 40:60 Compound A FB:HPMCAS-M | |
| Wet SDI, t = 24 hr | |
| 40:60 Compound A FB:HPMCAS-M, | |
| Discharged at ~36 hr | |

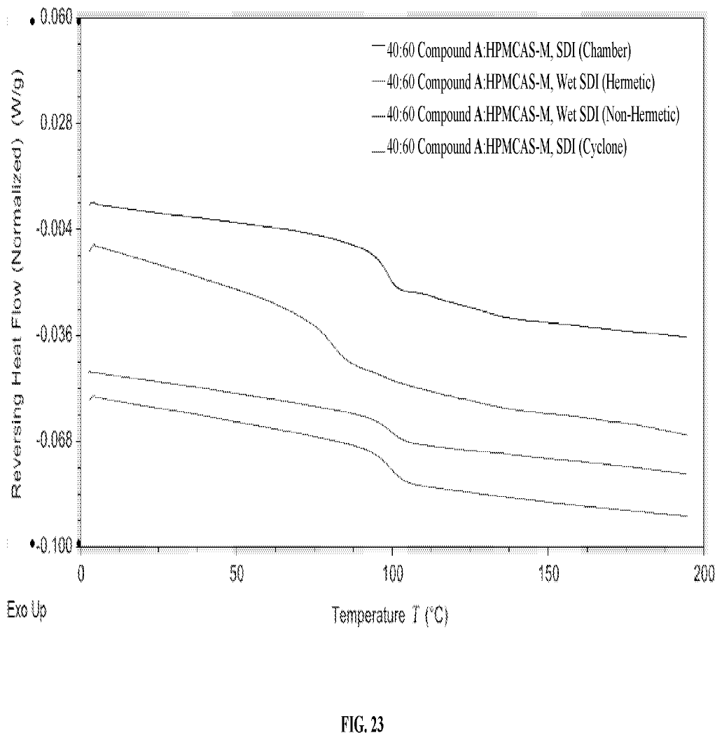

Thermal analysis done by MDSC was performed on the wet SDI with both hermetic and non-hermetic pans, to observe the suppression in Tg due to residual solvent content acting as a plasticizer, as well on the fully dried material and SDI harvested from the spray drying chamber. Hermetic results indicate the Tg was suppressed to 80° C. due to residual acetone, which is not a concerning level for physical stability. All other MDSC samples displayed Tg similar to previous data, FIG. 23 and Table 30 below.

TABLE 30
MDSC Data for Compound A Demonstration Batch SDI.

| | Measured |
|---|---|
| Formulations | Tg (° C.) |
| 40:60 Compound A:HPMCAS-M Wet SDI (Hermetic) | 80 |
| 40:60 Compound A:HPMCAS-M Wet SDI (Non-Hermetic) | 99 |
| 40:60 Compound A:HPMCAS-M Wet SDI (Cyclone) | 99 |
| 40:60 Compound A:HPMCAS-M Wet SDI (Chamber) | 99 |

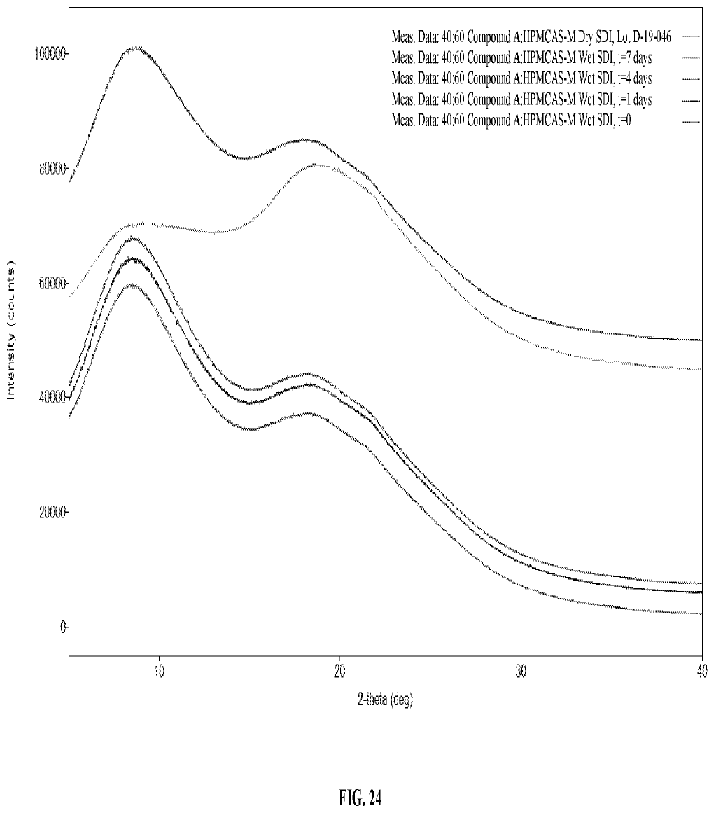

Characterization by XRPD was performed on the wet SDI also to observe any potential physical stability issues with prolonged exposure to high levels of acetone. Results indicate that the demonstration SDI remained an amorphous dispersion and no crystalline peaks were observed (FIG. 24).

Surface morphology of the demonstration batch wet SDI particles was characterized using scanning electron microscopy. Typical SDI morphology was observed consisting of whole and collapsed spheres with smooth surfaces. No crystalline material was observed in any sample throughout the drying curve indicating low level crystallization is not occurring in the presence of high acetone levels.

The spray solution stability of the demonstration batch SDI was performed over several time points at ambient temperature. No impurity growth was observed after 8 days in the spray solution, Table 31 below.

TABLE 31
Spray Solution Stability Data for Compound A Demonstration Batch SDI

| | Material | | | | |
|---|---|---|---|---|---|
| | Compound A FB | 40:60 Compound A FB:HPMCAS-M Spray Solution | | | |
| | Condition | | | | |
| | | RT, | RT, | RT, | RT, |
| RRT | RT | t = 0 | t = 2 days | t = 5 days | t = 8 days |
| 0.32 | Detected, | Detected, | Detected, | Detected, | Detected, |
| | <0.05% | <0.05% | <0.05% | <0.05% | <0.05% |
| 0.53 | Detected, | Detected, | Detected, | Detected, | Detected, |
| | <0.05% | <0.05% | <0.05% | <0.05% | <0.05% |
| 0.66 | 0.05% | 0.05% | Detected, | Detected, | Detected, |
| | | | <0.05% | <0.05% | <0.05% |
| 0.71 | Detected, | | Detected, | | |
| | <0.05% | | <0.05% | | |
| 0.77 | Detected, | | Detected, | | |
| | <0.05% | | <0.05% | | |
| 0.77 | Detected, | | | | |
| | <0.05% | | | | |
| 0.80 | Detected, | | | | |
| | <0.05% | | | | |
| 0.85 | Detected, | | | | |
| | <0.05% | | | | |
| 0.88 | | | | | |
| 0.93 | Detected, | Detected, | Detected, | Detected, | Detected, |
| | <0.05% | <0.05% | <0.05% | <0.05% | <0.05% |
| Total | 0.05% | 0.05% | 0.00% | 0.00% | 0.00% |
| Assay | 99.8 | 1.8 ± 0.0 | 1.9 ± 0.0 | 1.9 ± 0.1 | 1.9 ± 0.0 |
| (wt %) | | | | | |

The chemical stability of the wet SDI showed very similar results to the spray solution data, no observed impurity growth over a 7 day period, Table 32 below.

TABLE 32
Wet SDI Stability Data for Compound A Demonstration Batch SDI.

| | Material | | | | |
|---|---|---|---|---|---|
| | Compound A FB | 40:60 Compound A FB:HPMCAS-M Wet SDI | | | |
| | Condition | | | | |
| | | RT, | RT, | RT, | RT, |
| RRT | RT | t = 0 | t = 1 day | t = 4 days | t = 7 days |
| 0.32 | Detected, | Detected, | Detected, | Detected, | Detected, |
| | <0.05% | <0.05% | <0.05% | <0.05% | <0.05% |
| 0.53 | Detected, | Detected, | Detected, | Detected, | Detected, |
| | <0.05% | <0.05% | <0.05% | <0.05% | <0.05% |
| 0.66 | 0.05% | 0.05% | Detected, | Detected, | Detected, |
| | | | <0.05% | <0.05% | <0.05% |
| 0.71 | Detected, | Detected, | Detected, | | |
| | <0.05% | <0.05% | <0.05% | | |
| 0.77 | Detected, | Detected, | Detected, | Detected, | Detected, |
| | <0.05% | <0.05% | <0.05% | <0.05% | <0.05% |
| 0.77 | | Detected, | | Detected, | Detected, |
| | | <0.05% | | <0.05% | <0.05% |
| 0.80 | Detected, | Detected, | Detected, | Detected, | Detected, |
| | <0.05% | <0.05% | <0.05% | <0.05% | <0.05% |
| 0.82 | | | | | Detected, |
| | | | | | <0.05% |
| 0.85 | Detected, | Detected, | | 0.05% | 0.05% |
| | <0.05% | <0.05% | | | |
| 0.86 | | | | Detected, | Detected, |
| | | | | <0.05% | <0.05% |
| 0.88 | | Detected, | Detected, | Detected, | Detected, |
| | | <0.05% | <0.05% | <0.05% | <0.05% |
| 0.93 | Detected, | 0.05% | Detected, | 0.05% | Detected, |
| | <0.05% | | <0.05% | | <0.05% |
| 0.94 | | | | Detected, | Detected, |
| | | | | <0.05% | <0.05% |
| Total | 0.05% | 0.10% | 0.00% | 0.09% | 0.05% |
| Assay | 99.8 | 37.0 ± 2.9 | 39.0 ± 0.3 | 39.3 ± 0.1 | 39.5 ± 0.0 |
| (wt %) | | | | | |

The full characterization of the dry demonstration batch SDI was captured.

### 3.8. Oral Solid Dosage Form Development

### 3.8.1. Development of Prototype Tablet Formulations

The tablets were manufactured at 50 and 150 mg strengths. Optimization of the tablet formulation was done keeping in mind formulation variables: 0 SDI loading, disintegrant type and concentration, presence of a binding agent, type of filler used, and the grade of MCC used. The tablet quality attributes optimized during the product optimization were breaking force, disintegration time, compactibility, and compressibility.

Table 33 summarizes the formulation composition of the blends evaluated for feasibility.

TABLE 33
Compound A FB: HPMCAS-M Tablet Formulations*

| Lot# K9-983- | 22-F1 | 24-F3 | 24-F4 | 26-F5 | 26-F6 | 28-F7 | 29-F8 |
|---|---|---|---|---|---|---|---|
| Component | Content (w/w %) | | | | | | |
| 40:60 Compound A: | 55.00 | 55.00 | 65.00 | 65.00 | 65.00 | 65.00 | 65.00 |
| HPMCAS-M SDI | | | | | | | |
| MCC (PH-105) | 19.50 | 20.50 | 15.50 | 15.50 | 16.50 | — | 11.50 |
| MCC (PH-101) | — | — | — | — | — | 15.50 | — |
| Mannitol (Pearlitol 100 SD) | 19.50 | 20.50 | 15.50 | — | — | — | — |
| Lactose (Flow Lac 90) | — | — | — | 15.50 | 16.50 | 15.50 | 15.50 |
| Ac-Di-Sol | 4.00 | 2.00 | 2.00 | 2.00 | - | 2.00 | 2.00 |
| Cab-O-Sil | 1.00 | 1.00 | 1.00 | 1.00 | 1.00 | 1.00 | 1.00 |
| Sodium Stearyl Fumarate | 1.00 | 1.00 | 1.00 | 1.00 | 1.00 | 1.00 | 1.00 |
| HPC Nisso SSL SFP | — | — | — | — | — | — | 4.00 |
| Total | 100.00 | 100.00 | 100.00 | 100.00 | 100.00 | 100.00 | 100.00 |

* There was a formula calculation error in the manufacture of K9-983-22-F2; this data is not included.

In order to investigate the effect of adding a fraction of Ac-Di-Sol extragranularly, blend from lot #K9-983-26-F5 was further processed into blend lots K9-983-33-1 and K9-983-34-2 (Table 34) at 65 and 55% SDI loading. A lower breaking force vs. compression pressure trend was observed for K9-983-33-1, similar to the ones with higher (65%) SDI loading, along with faster disintegration times. Comparing % friability of these batches, it was observed that these tablets with higher SDI loading had high losses on friability testing (˜20%) at the lower compression forces. These results combined implied that increasing the SDI loading in the tablets resulted in tablets with weaker bonding forces. Thus, a 55% SDI loading was determined to be optimal for this formulation. In formulations K9-983-37 and K9-983-38, the complete addition of Ac-Di-sol intragranularly and addition of Kollidon CL-F as a disintegrating agent were investigated. Replacing the Ac-Di-Sol content to complete intragranular addition resulted in comparable disintegration times, however, replacement of the disintegrating agent to Kollidon CL-F resulted in a disproportionate increase in disintegration times with increase in compression force, with the tablet compressed at pressure 300 MPa not disintegrating at all.

TABLE 34
Compound A FB:HPMCAS-M Prototype Tablet Formulations

Lot # K9-983-

| | | | |
|---|---|---|---|
| 33-1 | 34-2 | 37 | 38 |

| | |
|---|---|
| Component | Content (w/w %) |

| | | | | |
|---|---|---|---|---|
| 40:60-Compound | 65.00 | 55.00 | 55.00 | 55.00 |
| A:HPMCAS-M SDI | | | | |
| MCC (PH-105) | 11.50 | 16.50 | 16.50 | 16.50 |
| FlowLac 90 | 15.50 | 20.50 | 20.50 | 20.50 |
| Ac-Di-Sol | 1.00 | 1.00 | 2.00 | — |
| Kollidon CL-F | — | — | — | 2.00 |
| Cab-O-Sil | 1.00 | 1.00 | 1.00 | 1.00 |
| SSF | 0.50 | 0.50 | 0.50 | 0.50 |
| Intragranular Total | 94.50 | 94.50 | 95.50 | 95.50 |
| MCC (PH-200) | 4.00 | 4.00 | 4.00 | 4.00 |
| Ac-Di-Sol | 1.00 | 1.00 | — | — |
| SSF | 0.50 | 0.50 | 0.50 | 0.50 |
| Extragranular Total | 5.50 | 5.50 | 4.50 | 4.50 |
| Total | 100.00 | 100.00 | 100.00 | 100.00 |

### 3.8.2. Scale-Up and Demonstration Batch Manufacturing of Lead Formulation

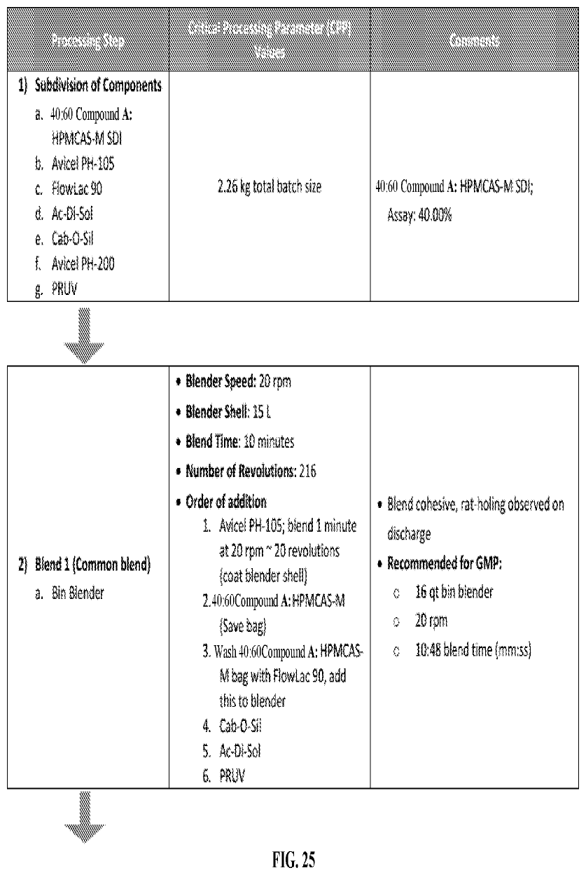

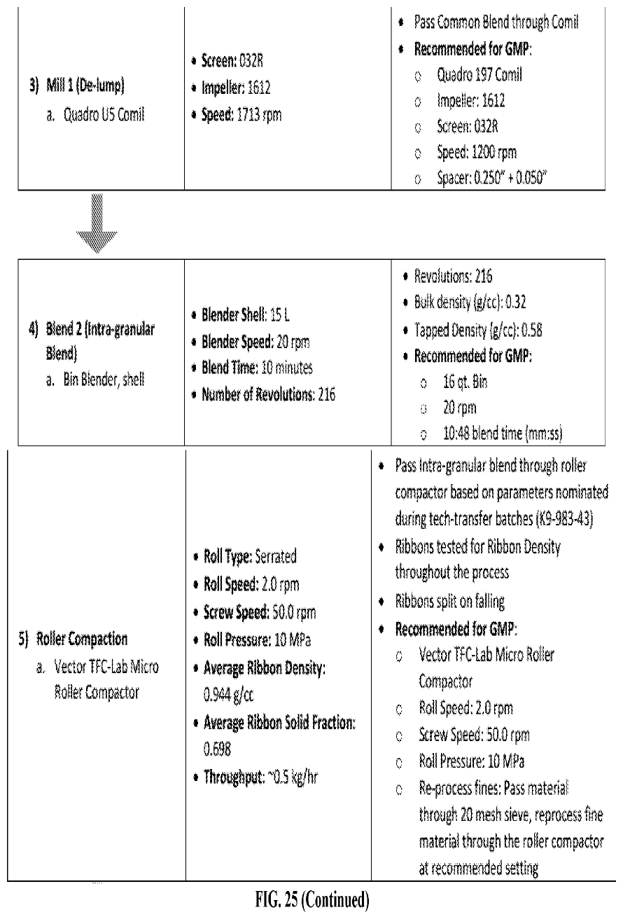

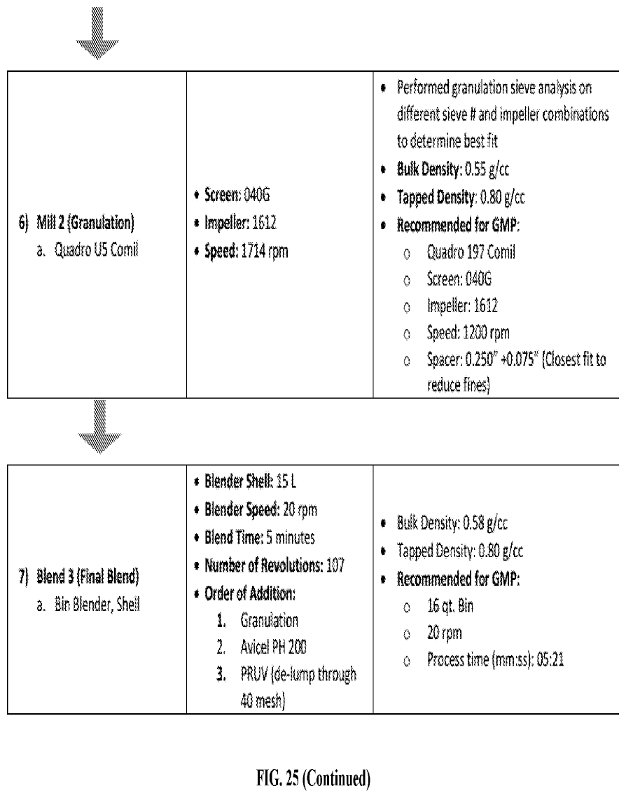

Based on the results from the feasibility batches, formulation blend K9-983-37 was scaled up onto the rotary press. A complete process flow chart for demonstration batch manufacturing of the common granulation is provided in FIG. 25.

Particle size analysis for the granulation and the final blend is listed in Table 35; the final blend had 19.18% fines. The dry granulation process improved the flow properties of the blends by reducing the Hausner ratio from 1.81 to 1.38 and densified the blend, increasing the bulk density from 0.32 to 0.58 g/cc (Table 36). Based on the final blend characterization, it was determined to be favorable for tablet compression.

TABLE 35
Compound A Demonstration Batch 220 mg/g Common Granulation
In-Process Particle Size Distribution

| | Mill 2 (Granulation) | Blend 3 (Final Blend) |
|---|---|---|
| Mesh/μm | % Retained | % Retained |
| 18/1000 | 0.39 | 0.41 |
| 20/841 | 0.79 | 0.41 |
| 30/595 | 19.72 | 18.37 |
| 40/420 | 20.07 | 17.14 |
| 60/250 | 16.84 | 16.33 |
| 120/125 | 11.83 | 13.88 |
| 200/74 | 9.07 | 14.29 |
| Pan | 21.29 | 19.18 |
| Total | 100.00 | 100.01 |

TABLE 36
Compound A Demonstration Batch 220 mg/g Common
Granulation In-Process Blend Characterization

| Step | LNB | Process | Bulk Density | Tap Density | Hausner |
|---|---|---|---|---|---|
| # | # | Step | (g/cc) | (g/cc) | Ratio |
| Blend 2 | K9-983-53 | Intragranular Blend | 0.32 | 0.58 | 1.81 |
| Mill 2 | K9-983-53 | Granulation | 0.55 | 0.80 | 1.45 |
| Blend 3 | K9-983-56 | Final Blend | 0.58 | 0.80 | 1.38 |

The master formula for the tablet strengths is given below in Table 37.

TABLE 37
Master Formula for Compound A Tablets,
50 mg and 150 mg. Lots: K9-983-58, 62

| | | Prototype & | GMP |
|---|---|---|---|
| | Compendial | Demo Batch | Batch |
| Component | Grade | w/w % | w/w % |
| 40:60 Compound A:HPMCAS-M SDI | Pharmaceutical | 55.00 | 55.00 |
| | (Patheon ORP) | | |
| Microcrystalline cellulose | USP/NF, EP | 16.50 | 16.50 |
| (Avicel PH-105) | | | |
| Lactose monohydrate. | USP/NF, EP | 20.50 | 20.50 |
| (Flow Lac 90) | | | |
| Colloidal Silicon Dioxide | USP/NF, EP | 1.00 | 2.00 |
| (Cab-O-Sil) | | | |
| Croscarmellose Sodium | USP/NF, EP | 2.00 | 1.00 |
| (Ac-Di-Sol) | | | |
| Sodium Stearyl Fumarate (PRUV) | USP/NF, EP | 0.50 | 0.50 |
| Intragranular Total | | | 95.50 |
| Microcrystalline cellulose | USP/NF, EP | 4.00 | 4.00 |
| (Avicel PH-200) | | | |
| Sodium Stearyl Fumarate | USP/NF, EP | 0.50 | 0.50 |
| Extragranular Total | | | 4.50 |
| Tablet Total | | | 100.00 |

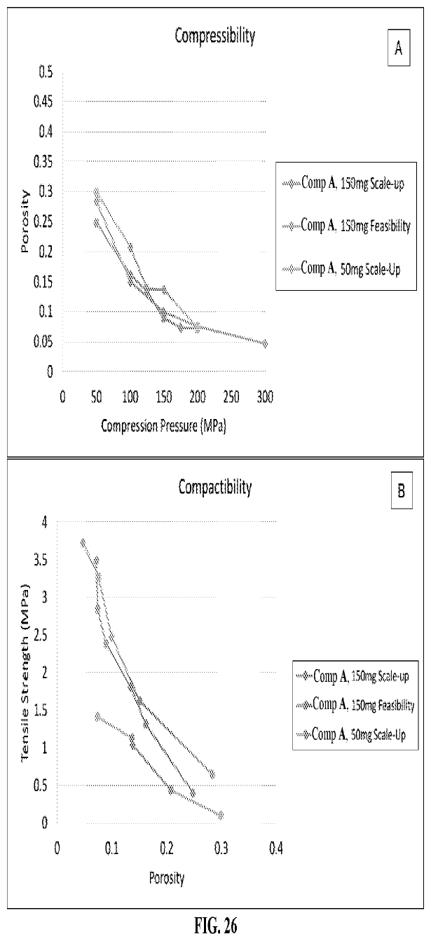

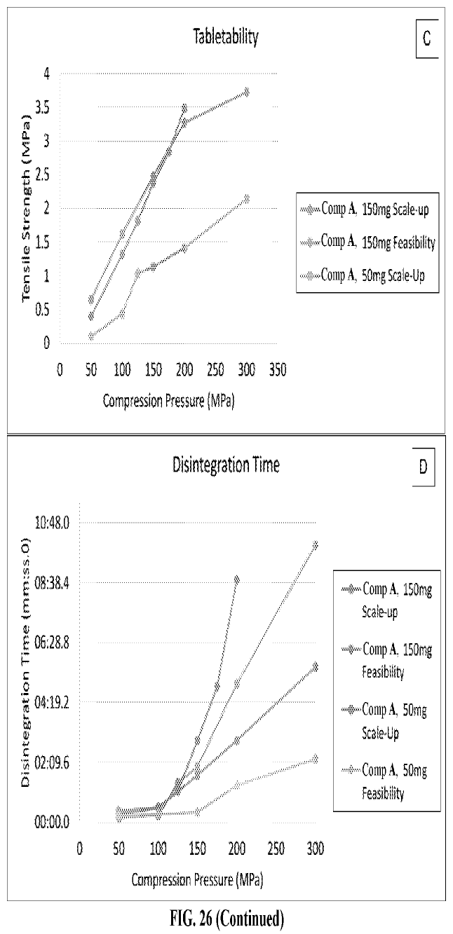

Scale-up batches were manufactured prior to the demonstration batch to generate tablet compression profiles for each tablet strength. FIG. 26 depicts the different graphs demonstrating compression profiles for both tablet strengths in comparison to the compression profile built for the lead feasibility batch (150 mg). Compressibility is the ability of the blend to undergo volume reduction under pressure. Looking at FIG. 26A, a steady decrease in tablet porosity is observed for both the tablet formulations on scale-up as well as for the feasibility batch up to a compression pressure of 200 MPa, depicting good blend compressibility. Compactability on the other hand, is the ability of the blend to be compressed into a compact of a specified strength. Looking at FIG. 26B, for the 150 mg tablets, the feasibility batch tablets at a lower tensile strength are slightly more porous than the scale-up batch. This can be attributed to differences in the manufacturing equipment used for the batches; a change from the single station press for the feasibility batch to the rotary press for the demonstration batch could result in a stronger compact by elimination of air pockets, improved particle rearrangement, and closer packing. The lines on the tabletability graph, plotting the tensile strength of the tablets at increasing compression pressure (FIG. 26C), overlap for both the feasibility and the scale-up batch for the 150 mg tablet strength.

Disintegration time increased for the tablets manufactured on the rotary press (for both tablet sizes) in comparison to those produced on the single station press. A change in the tooling for the larger sized tablet along with differences in the tablet presses could be responsible for the difference in the disintegration times observed.

Based on the compression profiles generated, a compression pressure of 150 MPa was nominated for the manufacture of tablets at both strengths. Tablets were monitored for weight (g), thickness (mm), and breaking force (kP) every 5 minutes throughout the process. A bulk sampling was conducted at the end of the batch for % friability and disintegration.

Overall characterization (Table 38) of the 50 mg tablets (K9-983-58) determined that compressing at a compression pressure of 150 MPa resulted in tablets with a disintegration time of ˜1 minute, breaking force of 10-13 kP, and friability of 0.00%. A compression pressure range of 125-175 MPa generated tablets with acceptable properties. For the 150 mg tablets, it was determined that the disintegration time of the tablets was sensitive to a change in the compression pressure; a drop in the disintegration times (to ˜1 min) was observed on compressing tablets around 125 MPa. Compressing tablets within a compression pressure range of 150-175 MPa generated tablets with acceptable tablet properties. These tablets had a disintegration time of 1-2 mins, breaking force of 23-26 kP, and friability of 0.029%.

TABLE 38
Composite testing for the Demonstration Manufacturing of Compound
A Tablets, 50 mg Lot: K9-983-58 & 150 mg Lot: K9-983-61

| Tablet | Tablet | Disintegration Time | | |
|---|---|---|---|---|
| strength, | Breaking | (mm:ss) (n = 6) | | % |
| Lot # | Force (kP) | Time First | Time Last | Friability |
| 50 mg, K9-983-58 | 9.8-13.4 | 00:40 | 01:02 | 0.00 |
| 150 mg, K9-983-61 | 23.3-26.4 | 01:02 | 01:41 | 0.029 |

### 3.9. Analytical Characterization of Compound A Tablets

The prototype tablet formulations were characterized by non-sink dissolution. The dissolution performance of the prototype tablets was tested in the non-sink dissolution test (FIG. 27 and Table 39). All tablets performed at a significantly lower dissolution level compared to their parent SDIs, especially the 50 mg tablets, indicating the tablet formulation is not allowing full release and subsequent dissolution of the SDI contained within the tablet.

TABLE 39
Non-Sink Dissolution Data for Compound A Prototype Tablets at 100 RPM

| | | Total | Total | Total | Total |
|---|---|---|---|---|---|
| | | Drug | Drug | Drug | Drug |
| | Lot | C_{maxGB} | C_{maxFaSSIF} | AUC_{35-210 FaSSIF} | C₂₁₀ |
| Sample | # | (μgA/mL) | (μgA/mL) | (min*μgA/mL) | (μgA/mL) |
| Compound A SDI | K9-983-45 | 2 | 67.4 | 8200 | 67.4 |
| prototype | | | | | |
| tablet, 50 mgA | | | | | |
| Compound A SDI | K9-983-47 | 8 | 108.5 | 15000 | 108.5 |
| prototype | | | | | |
| tablet, 150 mgA | | | | | |
| 40:60 Comp | K9-983-12 | 91 | 130.3 | 20000 | 130.3 |
| A:HPMCAS-M | | | | | |
| SDI | | | | | |

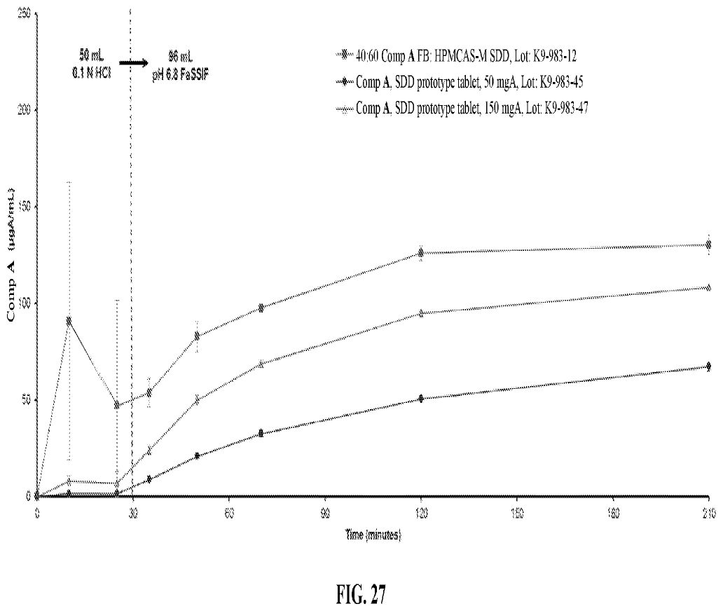

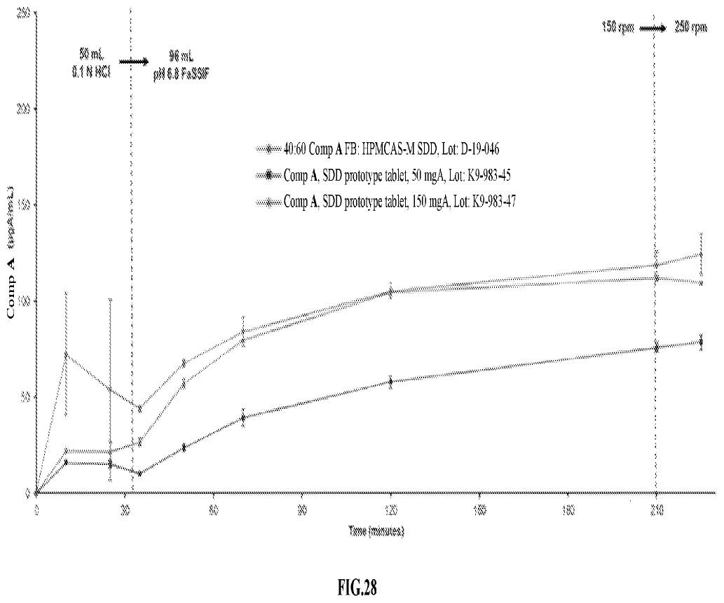

In an attempt to increase the drug release during non-sink dissolution testing, the paddle speed was increased to 150 RPM, and an infinity spin was added at the 210 min mark of 250 RPM, FIG. 28 and Table 40 below. This increase in paddle speed achieved a very similar dissolution profile for the 150 mg tablets, however the 50 mg tablets still remained quite low, with no observed effect occurring from the infinity spin.

TABLE 40
Non-Sink Dissolution Data for Compound
A Prototype Tablets at 150 → 250 RPM

| | | Total | Total | Total | Total |
|---|---|---|---|---|---|
| | | Drug | Drug | Drug | Drug |
| | Lot | C_{maxGB} | C_{maxFaSSIF} | AUC_{35-210 FaSSIF} | C₂₁₀ |
| Sample | # | (μgA/mL) | (μgA/mL) | (min*μgA/mL) | (μgA/mL) |
| 40:60 Compound | D-19-046 | 72 | 124.4 | 19000 | 124.4 |
| A FB:HPMCAS-M | | | | | |
| SDI | | | | | |
| Compound A SDI | K9-983-45 | 16 | 78.6 | 10500 | 78.6 |
| prototype | | | | | |
| tablet, 50 mgA | | | | | |
| Compound A SDI | K9-983-47 | 22 | 112.0 | 18000 | 109.7 |
| prototype | | | | | |
| tablet, 150 mgA | | | | | |

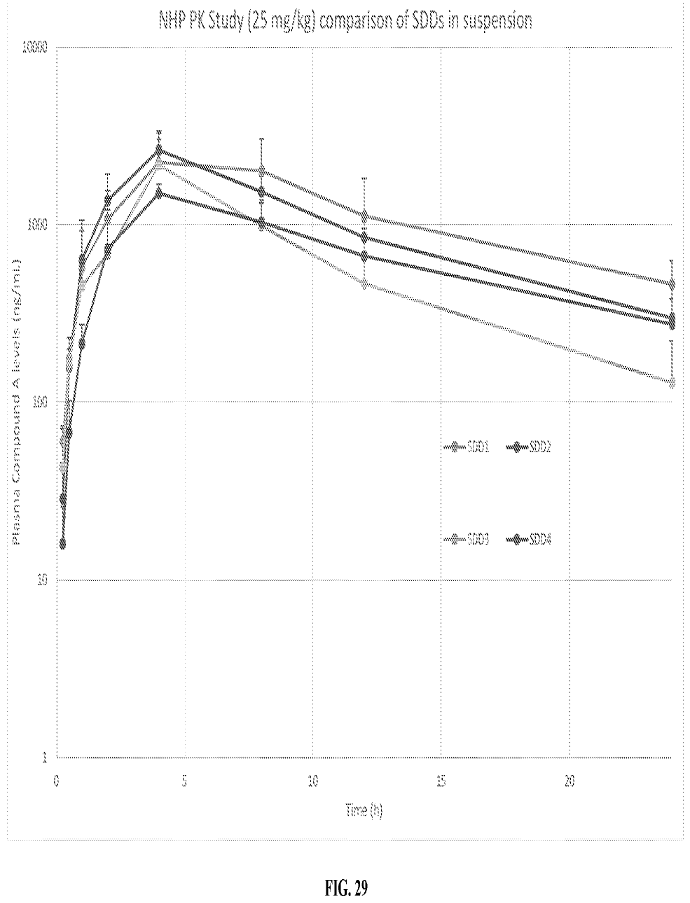

The SDDs provided good oral exposure in Cynomolgus Macaques. See Table 41 and FIG. 29.

TABLE 41
SDD Oral Exposure in Cynomolgus Macaques.

| | | Concentration (ng/mL) | | | | | |
|---|---|---|---|---|---|---|---|
| Group | Time | Animal 1* | Animal 2 | Animal 3 | AVE | SD | AUC0-24 (ng * h/mL) |
| SDD1 | 0.25 | 44.8 | 70.6 | 65.3 | 60 | 14 | 27658 |
| (25:70:5 | 0.5 | 167 | 183 | 175 | 175 | 8 | |
| Compound | 1 | 982 | 325 | 417 | 575 | 356 | |
| A:HPMCAS- | 2 | 1600 | 664 | 949 | 1071 | 480 | |
| L:TPGS) | 4 | 3430 | 1450 | 1850 | 2243 | 1047 | |
| | 8 | 2120 | 912 | 2980 | 2004 | 1039 | |
| | 12 | 986 | 494 | 1880 | 1120 | 703 | |
| | 24 | 292 | 480 | 618 | 463 | 164 | |
| SDD2 | 0.25 | 48.2 | 21.7 | 15.7 | 29 | 17 | 24055 |
| (40:60 | 0.5 | 205 | 138 | 131 | 158 | 41 | |
| Compound | 1 | 1130 | 397 | 378 | 635 | 429 | |
| A:HPMCAS- | 2 | 1980 | 870 | 1250 | 1367 | 564 | |
| M) | 4 | 3080 | 2460 | 2350 | 2630 | 394 | |
| | 8 | 1890 | 1190 | 1510 | 1530 | 350 | |
| | 12 | 1060 | 691 | 791 | 847 | 191 | |
| | 24 | 393 | 250 | 250 | 298 | 83 | |
| SDD3 | 0.25 | 75.6 | 25.3 | 28.2 | 43 | 28 | 15221 |
| (25:75 | 0.5 | 233 | 161 | 99 | 164 | 67 | |
| Compound | 1 | 651 | 354 | 354 | 453 | 172 | |
| A:HPMCP) | 2 | 719 | 601 | 732 | 684 | 72 | |
| | 4 | 3540 | 1570 | 1410 | 217 | 1186 | |
| | 8 | 1380 | 800 | 765 | 982 | 345 | |
| | 12 | 720 | 331 | 348 | 466 | 220 | |
| | 24 | 233 | 57.7 | 96.2 | 129 | 92.1 | |
| SDD4 | 0.25 | 27.5 | 9.54 | 10.9 | 16 | 10 | 16297 |
| (40:60 | 0.5 | 105 | 36.4 | 61.5 | 68 | 35 | |
| Compound | 1 | 282 | 164 | 194 | 213 | 61 | |
| A:HPMCP) | 2 | 1290 | 394 | 509 | 731 | 488 | |
| | 4 | 1610 | 1610 | 1280 | 1500 | 191 | |
| | 8 | 1070 | 1360 | 667 | 1032 | 348 | |
| | 12 | 924 | 716 | 363 | 668 | 284 | |
| | 24 | 505 | 189 | 130 | 275 | 202 | |

### 4. Conclusions

The Compound A SDIs and tablets should provide significantly enhanced in vivo exposure compared to the crystalline API based on the physiochemical characterization and in vitro dissolution performance testing. Both 50 mg and 150 mg tablets containing 40:60 Compound A:HPMCAS-M SDI were successfully formulated into immediate release tablets. In vivo studies and clinical trials will be performed to assess the efficacy of the formulations.

### Example 2: Non-Clinical Studies Demonstrating Potency and Efficacy of Compound A Alone and in Combination with a PDx Inhibitor

### Nonclinical Pharmacology

### In Vitro Pharmacology

A series of cellular assays in cell lines and in primary immune cells were conducted to determine the potency and mechanism of action of Compound A.

### In Vitro Activity of Compound A in Mouse and Rat Cell Lines

The ability of Compound A to inhibit AHR-dependent Cyp1A1 gene expression was examined in vitro by measuring changes in Cyp1A1 enzymatic activity in 2 rodent hepatoma cell lines following AHR agonist stimulation. Mouse Hepa1.6 and rat H411E hepatoma cells were treated with AHR agonists VAF347 and L-kynurenine, respectively, in the presence of Compound A at multiple concentrations for 24 hours. The inhibition of Cyp1A1 expression was subsequently evaluated by measuring Cyp1A1 enzyme activity using P450-Glo assays. In murine Hepa1.6 cells treated with 2 μM VAF347, Compound A inhibited AHR-dependent expression of Cyp1A1 in a concentration-dependent manner with an average IC50 of 36 nM. In rat hepatoma H411E cells treated with 100 μM L-kynurenine, Compound A inhibited AHR-dependent Cyp1A1 expression in a concentration-dependent manner with an IC50 of 151 nM.

### In Vitro Activity of Compound A and Metabolites in a Human Cell Line

In vitro experiments were conducted to examine the inhibitory activity of Compound A for AHR-mediated transcriptional activation in the HepG2 DRE-Luc reporter cell line. This human hepatoma cell line stably expresses a luciferase reporter gene under control of AHR-responsive DRE enhancer elements (Han, 2004). HepG2 DRE-Luc reporter cells were treated with 80 nM VAF347 to activate AHR. Compound A inhibited VAF347-stimulated luciferase expression in a concentration-dependent manner with an IC50 of 91 nM (n=2).

The inhibitory activity of the human Compound A metabolites, Compound B and Compound C was also determined in the HepG2 DRE-Luc cell line. Reporter cells were stimulated with 80 nM VAF347 and each metabolite at multiple concentrations. Both Compound A metabolites were shown to effectively inhibit AHR-dependent luciferase expression in a concentration-dependent manner. The IC50 for Compound B was 23 nM while the IC50 for Compound C was 213 nM (n=2 for both).

### In Vitro Activity of Compound A in Cynomolgus Macaque Peripheral Blood Mononuclear Cells

The effect of Compound A on AHR-dependent gene expression was assessed in peripheral blood mononuclear cells (PBMCs) of cynomolgus macaque monkeys to assess activity in the non-rodent tox species. Cynomolgus PBMCs were treated ex vivo with Compound A and gene expression of AHR-dependent genes CYP1B1 and AHR was quantified using Quantigene Plex (QGP) custom panels. Compound A inhibited the AHR target genes Cyp1B1 and AHR in a concentration-dependent manner with IC50 values of 6 and 30 nM, respectively, demonstrating AHR inhibition in PBMCs of a nonhuman primate species.

### In Vitro Activity of Compound A in Human T Cells and Whole Blood

AHR plays a key role in immune cells and its' inhibition is proposed to reverse immune suppression and activate T cells. The ability of Compound A to inhibit AHR-dependent CYP1A1 expression and cytokine production was assessed in primary human T cells. AHR directly regulates the expression of the immune suppressive cytokine IL-22. Human T cells isolated from healthy donor PBMCs were activated with CD3/CD28 tetramer and incubated for 24 hours with Compound A. Cell pellets were processed for RNA isolation and CYP1A1 analysis by quantitative reverse-transcriptase polymerase chain reaction. For the cytokine analysis assay, CD3/CD28 activated T cells were treated with Compound A, and culture supernatants were collected after 48 hours for analysis of IL-22 levels using Meso Scale Discovery V-plex IL-22 plates. Compound A inhibited AHR-dependent gene expression in activated human T cells by decreasing expression of CYP1A1 in a concentration-dependent manner. The IC50 was determined to be 63 nM. Compound A also inhibited IL-22 secretion by activated T cells in a concentration-dependent manner, with an IC50 value of 7 nM.

To further examine the effects of Compound A on basal and ligand-activated AHR-dependent gene expression in human immune cells, blood samples from 2 healthy human donors were exposed ex vivo to Compound A in the presence or absence of 20 μM L-kynurenine to activate AHR. After 24 hours, cells were evaluated for CYP1B1 gene expression. In whole blood samples without AHR activation, basal levels of CYP1B1 expression were inhibited by Compound A treatment in both donors (FIG. 30A). Compound A also inhibited AHR ligand L-kynurenine-induced CYP1B1 in treated whole blood from 2 different donors (FIG. 30B). In both donors, Compound A concentrations >0.5 μM inhibited CYP1B1 gene expression by greater than 50% under basal and ligand activated conditions.

### In Vivo Pharmacology

Activation of AHR by kynurenine or other ligands alters gene expression of multiple immune modulating genes leading to immunosuppression within both the innate and adaptive immune system (Opitz, 2011). This AHR-mediated immune suppression plays a role in cancer since its activity prevents immune cell recognition of and attack on growing tumors (Murray, 2014; Xue, 2018; Takenaka, 2019). In vivo studies were performed with Compound A to demonstrate the on-target inhibition of AHR in pharmacodynamic studies and in TGI in multiple tumor models as a single agent, and in combination with the checkpoint inhibitor anti-PD-1.

### Pharmacodynamics of Compound A in Murine Liver and Spleen

The pharmacodynamic effect of Compound A on the inhibition of AHR-dependent gene expression in liver and spleen was examined in C57BL/6 mice. In this study, AHR was activated by oral dosing of mice with VAG539, a pro-drug of the active agonist VAF347 (Hauben, 2008).

C57BL/6 female mice were treated by oral gavage with vehicle or the AHR agonist VAG539 at 30 mg/kg. In some mice, Compound A oral dosing at 5, 10, and 25 mg/kg was immediately followed by administration of VAG539. Mice were sacrificed at 4 and 10 hours post-dose and RNA was extracted and gene expression of CYP1A1 and the housekeeping gene mouse glyceraldehyde 3-phosphate dehydrogenase were quantified. CYP1A1 mRNA expression levels for each dose group for liver and spleen tissues were normalized to the control group.

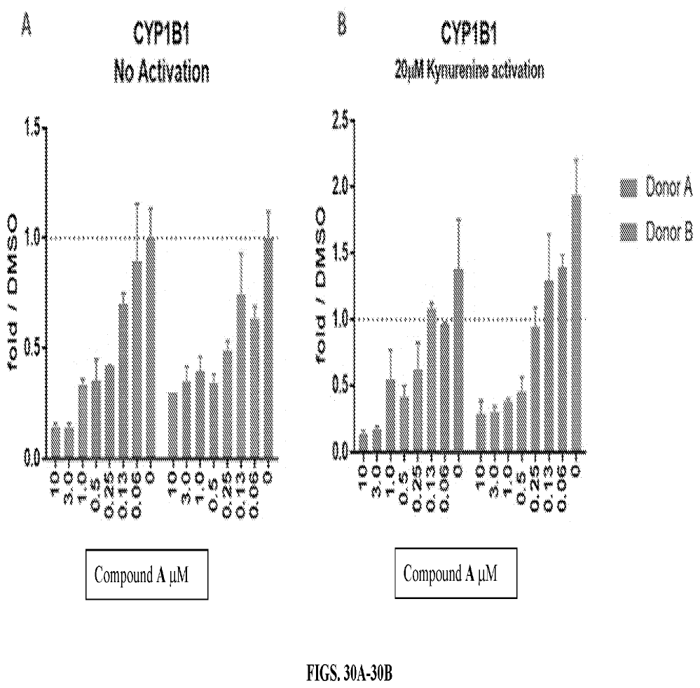

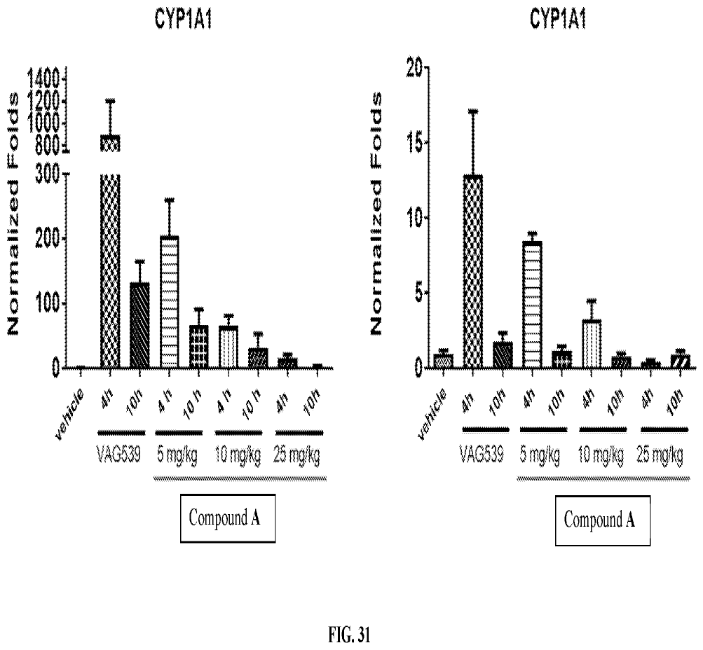

Following administration of 30 mg/kg VAG539 alone, AHR-dependent CYP1A1 expression in the liver was increased 895-fold 4 hours and 132-fold 10 hours post-treatment. The increased expression of CYP1A1 mRNA in the liver was inhibited in a dose-dependent manner by coadministration with Compound A (FIG. 2). Complete inhibition of CYP1A1 mRNA increases induced by VAG539 was observed with a dose of 25 mg/kg Compound A. The induction of CYP1A1 expression by VAG539 was lower in the mouse spleen, with increases of 12.9-fold 4 hours and 1.8-fold 10 hours post-treatment. Coadministration of Compound A with VAG539 led to dose-dependent inhibition of CYP1A1 mRNA induction in the spleen, with complete inhibition achieved at 4 hours when mice were treated with 25 mg/kg Compound A (FIG. 31). This study demonstrates dose-dependent and on-target inhibition of AHR by Compound A in the mouse liver and spleen.

Activity of Compound A in Combination with Anti-PD-1 Antibody (BioXcell RMP1-14) in the B16-IDO1 Orthotopic Mouse Melanoma Cancer Model

The effect of Compound A treatment alone and in combination with an anti-PD-1 antibody (BioXcell RMP1-14) on tumor growth was determined in a C57Bl/6 mouse syngeneic model of orthotopic melanoma. B16-F10 murine melanoma tumor cells were engineered to overexpress IDO1, known to catabolize tryptophan into kynurenine, thereby activating the AHR (Holmgaard, 2015).

C57Bl/6 female mice were inoculated intradermally with B16-IDO1 tumor cells. Once tumors were established, animals were treated with vehicle, Compound A, anti-PD-1 antibody, or a combination of anti-PD-1 antibody and Compound A. Compound A (25 mg/kg) was administered orally once daily (QD) for 12 days, while anti-PD-1 antibody (250 μg/mouse) was administered intraperitoneal (IP) every 3 days for a total of 5 doses.

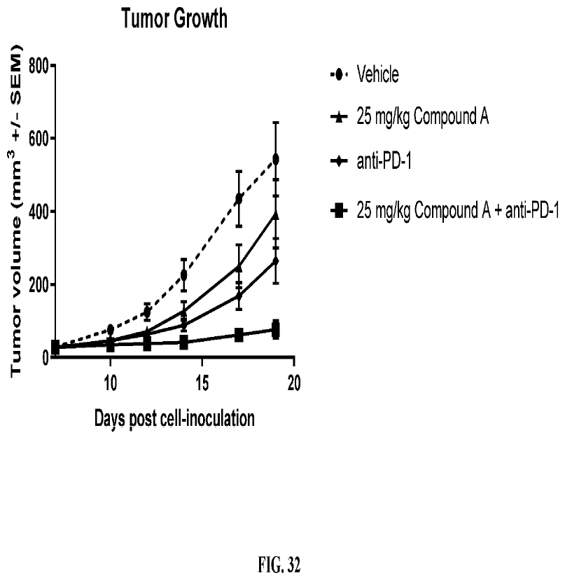

Administration of anti-PD-1 antibody resulted in a TGI of 51.4% (p=0.025) compared to the vehicle control group. The combination of Compound A and anti-PD-1 antibody resulted in a significant TGI of 86% (p=0.0001) compared to vehicle and 71.2% (p=0.0109) compared to the anti-PD-1 antibody monotherapy group which led to 1 CR (FIG. 32). These data demonstrate a combined effect of Compound A and anti-PD-1 antibody on TGI in a murine model of melanoma.

Effect of Compound A Alone and in Combination with Anti-PD-1 Antibody (BioXcell RMP1-14) on Tumor Growth and Host Survival in Mice Bearing the CT26.WT Murine Colorectal Cancer Model

The effect of single agent Compound A, and Compound A in combination with anti-PD-1 antibody (BioXcell RMP1-14) on TGI and tumor survival was evaluated in the CT26.WT syngeneic model of colorectal cancer. Balb/cJ female mice were inoculated subcutaneously with tumor cells and 4 days after inoculation, Compound A (10 mg/kg or 25 mg/kg) or Vehicle was administered orally QD for a total of 53 doses. Concurrently, anti-PD-1 antibody (10 mg/kg) was administered IP twice a week for a total of 5 doses.

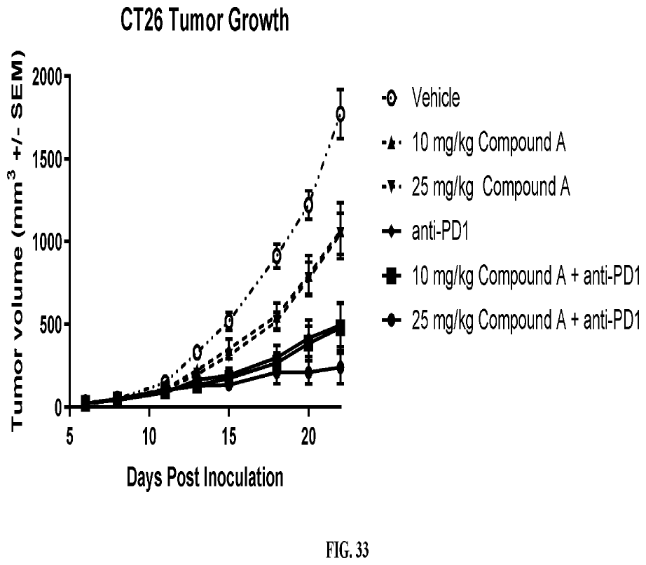

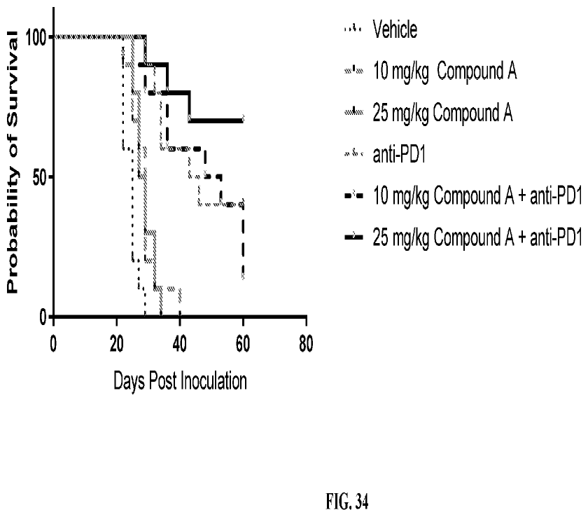

Compound A as a single agent resulted in significant TGI as compared to the vehicle control group. The oral administration of 10 and 25 mg/kg Compound A resulted in TGI of 39.8% (p=0.0061) and 40.9% (p=0.0015), respectively, relative to vehicle treated mice. The IP administration of anti-PD-1 antibody resulted in a TGI of 72.1% (p≤0.0001) relative to vehicle treated mice. The combination of 10 mg/kg or 25 mg/kg Compound A and anti-PD-1 antibody resulted in a significant TGI of 72.9% (p≤0.0001) and 86.5% (p≤0.0001), respectively, relative to vehicle treated mice. (FIG. 33). The combination of 25 mg/kg Compound A with anti-PD-1 antibody resulted in complete responses (CRs) in 7 out of 10 mice (tumor re-challenge was initiated at >95 days post CR determination), whereas anti-PD-1 antibody as a monotherapy resulted in 4 CRs. Consequently, the combination of 25 mg/kg Compound A with anti-PD-1 antibody showed a survival benefit over anti-PD-1 antibody monotherapy (FIG. 34). The combination of 10 mg/kg Compound A with anti-PD-1 antibody also resulted in CRs in 2 mice.

At ≥95 days after the appearance of CRs in mice treated with the combination of Compound A and anti-PD-1 antibody, the responder animals were re-challenged with CT26.WT cells. Five naïve mice were also injected with CT26.WT cells as a positive control for tumor formation. Twenty-one days after cell inoculation, all naïve mice had tumors, yet no tumor growth was detected in the CR mice from the anti-PD-1 antibody alone group or the 10 mg/kg Compound A and anti-PD-1 antibody groups. In the 25 mg/kg Compound A and anti-PD-1 antibody group, 1 CR had a small tumor (>104 mm³) and 6 out of 7 CRs did not have any tumor detectable tumor growth, demonstrating the presence of T cell memory cells against CT26.WT cells.

These studies indicate that the anti-tumor activity of Compound A synergizes with and enhances the activity of immune checkpoint blockade inhibitors.

### Example 3. A Phase 1, Open-Label, Dose-Escalation and Expansion Study of Compound A, an Oral Aryl Hydrocarbon Receptor (AHR) Inhibitor in Patients with Locally Advanced or Metastatic Solid Tumors and Urothelial Carcinoma

### 1. Objectives:

### Primary:

To determine the maximum tolerated dose (MTD) and to characterize the dose-limiting toxicities (DLTs) of Compound A
To evaluate additional safety and tolerability of Compound A, including acute and chronic toxicities, in determining a recommended phase 2 dose (RP2D) of Compound A

### Secondary:

To evaluate and characterize the PK of Compound A and any major active metabolites
To evaluate disease response with Compound A treatment
To evaluate pharmacodynamic immune effects of Compound A in collected paired tumor biopsies

### Exploratory:

To evaluate the pharmacodynamic effects of Compound A on AHR target gene expression in paired blood draws and paired tumor biopsies
To evaluate the pharmacodynamic effects of Compound A on peripheral immune cell and chemokine/cytokine in paired blood draws
To assess candidate baseline biomarkers in tumor or blood to better understand the relationship between Compound A treatment and response, or resistance.

### 2. Endpoints:

### Primary:

Identification of a dose that is deemed acceptable per the modified Toxicity Probability Interval (mTPI-2) design
Safety endpoint: Frequency of adverse events (AEs) overall, by grade, relationship to study treatment, time-of-onset, duration of the event, duration of resolution, and concomitant medications administered

### Secondary:

Determination of Compound A PK parameters, including half-life (t½), area under the plasma concentration-time curve (AUC) and maximum observed plasma concentration (Cmax)
Preliminary antitumor activity endpoints per RECIST 1.1: Objective response rate (ORR), progression-free survival (PFS), duration of treatment (DOT), disease control rate (DCR), duration of response (DOR). For patients with urothelial carcinoma, at the Investigator's discretion, additional antitumor endpoints include assessment per iRECIST
Immune pharmacodynamic endpoints: including but not limited to the characterization of tumor infiltrating cytotoxic T cells in tumor biopsies collected before and during Compound A treatment.

### Exploratory:

Changes in AHR target gene expression in blood cells and tumor tissues after study drug treatment
Changes in immune cell types, including but not limited to circulating helper T cells, cytotoxic T cells, and regulatory monocytes after study drug treatment
Correlation of baseline tumor biomarkers, including but not limited to AHR, IDO1, and TDO2 protein expression, AHR target gene expression, and gene expression profiling of immune response

### Study Design

This is a first-in-human (FIH), single-arm, dose-escalation and expansion study to evaluate the safety, tolerability, PK, pharmacodynamics, and preliminary antitumor activity of Compound A administered orally in patients with advanced solid tumors and urothelial carcinoma. Subject enrollment and continuous safety assessment will be guided by a mTPI-2 design (Guo, 2017). Decisions for dose escalation and de-escalation will be made by a Safety Review Committee (SRC) comprised of the enrolling study Investigators and the Sponsor. To assess evidence of preliminary antitumor activity, a Simon 2-stage design (Simon, 1989) is used.

A 28-day baseline Screening period (Day −28 to Day −1; including a 14-day Screening period for tumor scanning assessments and, in some instances, a pre-treatment biopsy) is followed by a by a Single-dose Run-in period (up to 7 days) to assess the PK of Compound A without food. For the purposes of the Single-dose Run-in period, unless otherwise indicated by or discussed with the Sponsor, the fasted state is defined as no solid food or liquids, except water and medication, from midnight of the night preceding the single dose to 2 hours after taking the Compound A. During the Treatment period, Subjects are instructed to consume a meal containing ≥6 grams of fat prior to taking Compound A daily, but should otherwise maintain a normal diet. The treatment arm comprises a daily oral administration of Compound A in the fed state. There will be no planned interruptions in this schedule. However, for the purpose of scheduling various evaluations during the study, 3 weeks of treatment (i.e., every 21 days) will correspond to 1 cycle of therapy. Subjects may continue treatment until disease progression, unacceptable toxicity, or consent withdrawal. At a minimum, the 30-Day and 90-Day Follow-up visits should occur 30 days and 90 days (±7 days), respectively, after the last study drug administration. If an alternate therapy initiates during this period, the 30-Day and/or 90-Day Follow-up visits should be conducted prior to the first dose of alternate therapy.

Archival tumor tissue can be collected to explore tumor AHR nuclear localization as a predictive biomarker for disease response to Compound A in patients with urothelial carcinoma. Patients with urothelial carcinoma can consent to the AHR nuclear localization assessment prior to the Screening period. Preference is given to those patients whose assessment is positive. There is no time limit (i.e., window) for this assessment during the Prescreening period. Archival tumor tissue should be used within 1 year of accessioning unless otherwise discussed with the Sponsor.

Toxicity is evaluated according to National Cancer Institute Common Terminology Criteria for Adverse Events (AEs) (NCI-CTCAE) v5.0. DLT events are defined herein. AEs will be assessed, and laboratory values (chemistry, hematology, coagulation, thyroid function and urinalysis as specified herein), vital signs, and 12-lead triplicate electrocardiograms (ECGs) are obtained to evaluate the safety and tolerability of Compound A.

A modified Toxicity Probability Interval (mTPI-2) design (Guo, 2017) with a target DLT rate of approximately 30% is applied for dose escalation and confirmation to determine the Compound A RP2D. Several dose levels of Compound A, planned from 200 mg QD to 1600 mg daily, are explored. De-escalation doses of Compound A are also available if the starting dose is deemed intolerable. All dose escalation and de-escalation decisions will be based on the occurrence of DLTs at a given dose during the first 21-day period (Cycle 1) and will be made by the SRC. At any time DLT events pass an unacceptable toxicity threshold, the dose of Compound A is lowered for all subjects being treated at that dose level. If a subject is benefiting and is without severe treatment-emergent adverse events (TEAEs), that subject may be permitted to receive additional doses of Compound A at the same dose after discussion between the Investigator and the Sponsor.

During dose escalation, a minimum of 3 patients are required at each dose. Depending on accrual rate and occurrence of DLTs, 3, 4, 5, or 6 patients may be enrolled at each new dose until the last of those patients completes the 21-day DLT assessment period. Based on the mTPI-2 design, the number of patients who are enrolled at a dose but are not yet fully evaluable for DLT assessment may not exceed the number of remaining patients who are at risk of developing a DLT before the dose would be considered unacceptably toxic. In general, 3 to 14 patients can be enrolled at a given dose level. Administration of Compound A to the first 2 patients in each new dose cohort is staggered by a minimum of 15 hours. At any time Compound A plasma exposures approach levels at or within 75% of a Cmax of 11,200 ng/mL or an AUC of 188,000 ng*h/mL where QTc increases are noted in primates (i.e., Cmax of 8,400 ng/mL or AUC of 141,000 ng*h/mL), dose-escalation steps are limited to 50% of the previous dose.

Dose escalation and safety confirmation expansion end after 14 patients have been treated at any of the selected doses found to be acceptable. The totality of the data is considered before a dose is selected to carry forward and the escalation schedule may be adjusted based on PK, pharmacodynamics, and safety data emerging throughout the study to determine the RP2D.

The subject population used for determining the MTD comprises subjects who have met the minimum safety evaluation requirements of the study and/or who have experienced a DLT.

Serial blood samples are obtained to characterize the plasma PK of Compound A and its major active metabolites. The initial sampling strategy is based on the predicted human PK of this compound. If in the course of evaluating the PK, it is determined that an alternative sampling scheme would be more informative, then that alternative sampling scheme may be implemented if the total amount of blood and blood draws obtained for PK is not increased. Moreover, the total number of samples may be decreased at any time if the initial sampling scheme is considered unnecessarily intensive.

Because the starting dose and any higher dose is expected to be near or at the pharmacologically active range, each subject is required to have blood drawn and tumor biopsies for secondary and exploratory pharmacodynamic endpoints. The blood and tumor tissue samples are used to confirm AHR target engagement. Individual subjects can be exempted from the tumor biopsy requirement upon discussion and prior agreement by the Sponsor. The initial sampling strategy is based on the predicted human pharmacodynamics of this compound. If in the course of evaluating the pharmacodynamics, it is determined that an alternative sampling scheme would be more informative, then that alternative sampling scheme can be implemented if the total amount of blood, blood draws, and tumor biopsies obtained for pharmacodynamics is not increased. Moreover, the total number of samples can be decreased at any time if the initial sampling scheme is considered unnecessarily intensive.

Although the primary endpoints of this study are safety and tolerability, preliminary antitumor activity that may be associated with Compound A is assessed by measuring changes in tumor size by computed tomography (CT) or magnetic resonance imaging (MRI). Tumor assessment is performed after the completion of every 8 weeks of treatment for the first 6 months using Response Evaluation Criteria Solid Tumors version 1.1 (RECIST 1.1), unless there is progression based on clinical signs and/or symptoms. For subjects with urothelial carcinoma, additional tumor assessments may be performed per immune RECIST (recast) at the discretion of the Investigator. Subjects receiving more than 6 months of therapy have tumor assessments performed routinely after the completion of every 12 weeks of treatment.

To assess evidence of preliminary antitumor activity in patients with urothelial carcinoma, a Simon 2-stage design (Simon, 1989) is used. It is anticipated that 11 to 14 subjects of the 14 subjects treated at the preliminary RP2D will have urothelial carcinoma, however additional subjects may be enrolled to enable a minimum of 11 efficacy evaluable subjects with urothelial carcinoma as needed. There would need to be at least 1 response in these initial 11 to 14 subjects with urothelial carcinoma to proceed to the second stage in which additional subjects with urothelial carcinoma will be enrolled to complete a 28 subject cohort. A total of 4 responses among these 28 subjects would indicate further study of the drug is warranted based on this design in this population of subjects at alpha=0.05, 1-sided, excluding the null hypothesis of a response rate of 0.05 or less. The expected response rate is 0.20. The power for this design is approximately 0.80 to 0.83. Based on expected enrollment rates, the Sponsor may elect not to pause enrollment between Stage 1 and Stage 2.

### Main Criteria for Inclusion:

1. Patients ≥18 years of age.

2. Patients with histologically confirmed solid tumors who have locally recurrent or metastatic disease that has progressed on or following all standard of care therapies deemed appropriate by the treating physician, or who is not a candidate for standard treatment.

3. For patients with urothelial carcinoma to be enrolled in the dose expansion phase, patients must have histological confirmation of urothelial carcinoma and have unresectable locally recurrent or metastatic disease that has progressed on or following all standard of care therapies deemed appropriate by the treating physician (e.g., including a platinum-containing regimen and checkpoint inhibitor), or who is not a candidate for standard treatment. There is no limit to the number of prior treatment regimens.

4. Have measurable disease per RECIST v1.1 as assessed by the local site Investigator/radiology. Lesions situated in a previously irradiated area are considered measurable if progression has been demonstrated in such lesions.

5. Tumor can be safely accessed for multiple core biopsies and patient is willing to provide tissue from available archival and newly obtained biopsies before and during treatment, unless discussed with Sponsor.

6. Time since the last dose of prior therapy to treat underlying malignancy (including other investigational therapy):

a. Systemic cytotoxic chemotherapy: ≥the duration of the most recent cycle of the previous regimen (with a minimum of 2 weeks for all, except 6 weeks for systemic nitrosourea or systemic mitomycin-C);

b. Biologic therapy (e.g., antibodies): ≥3 weeks;

c. Small molecule therapies: ≥5× half-life.

7. Have an Eastern Cooperative Oncology Group (ECOG) performance status of 0 to 1.

8. Adequate organ function as follows. Specimens must be collected within 7 days prior to the start of study treatment.

a. Absolute neutrophil count (ANC)≥1500/μL;

b. Hemoglobin >8 g/dL;

c. Platelet Count >80,000/μL;

d. Serum creatinine ≤1.5× upper limit of normal (ULN) or creatinine clearance ≥50 mL/min for patients with creatinine levels >1.5× institutional ULN (using the Cockcroft-Gault formula);

e. Serum total bilirubin ≤1.5×ULN or direct bilirubin ≤ULN for patients with total bilirubin levels >1.5×ULN;

f. Aspartate aminotransferase (AST) and alanine aminotransferase (ALT)≤2.5×ULN (or ≤5×ULN if liver metastases are present);

g. Coagulation: ≤1.5×ULN unless subject is receiving anticoagulant therapy as long as PT or aPTT is within therapeutic range of intended use of anticoagulants.

9. Highly effective contraception for both male and female patients if the possibility of conception exists.

10. Patient able and willing to provide written informed consent and to comply with the study protocol and with the planned surgical procedures.

### Main Criteria for Exclusion

1. Clinically unstable central nervous system (CNS) tumors or brain metastasis (stable and/or asymptomatic CNS metastases allowed).

2. Patients who have not recovered to ≤Grade 1 or baseline from all AEs due to previous therapies (patients with ≤Grade 2 neuropathy may be eligible after discussion with the Sponsor).

3. Has an active autoimmune disease that has required systemic treatment in past 2 years with the use of disease-modifying agents, corticosteroids, or immunosuppressive drugs; nonsteroidal anti-inflammatory drugs (NSAIDs) are permitted.

4. Any condition requiring continuous systemic treatment with either corticosteroids (>10 mg daily prednisone equivalents) or other immunosuppressive medications within 2 weeks prior to first dose of study treatment (Inhaled or topical steroids and physiological replacement doses of up to 10 mg daily prednisone equivalent are permitted in the absence of active clinically significant [i.e., severe] autoimmune disease.).

5. Any other concurrent antineoplastic treatment or investigational agent except for allowed local radiation of lesions for palliation (to be considered non-target lesions after treatment) and hormone ablation.

6. Uncontrolled or life-threatening symptomatic concomitant disease (including known symptomatic human immunodeficiency virus (HIV), symptomatic active hepatitis B or C, or active tuberculosis).

7. Has undergone a major surgery within 3 weeks of starting trial treatment or has inadequate healing or recovery from complications of surgery prior to starting trial treatment.

8. Has received prior radiotherapy within 2 weeks of start of study treatment. Subjects must have recovered from all radiation-related toxicities, not require corticosteroids, and not have had radiation pneumonitis. A 1-week washout is permitted for palliative radiation [≤2 weeks of radiotherapy] to non-CNS disease.

9. Prior AHR inhibitor treatment without Sponsor permission.

10. Potentially life-threatening second malignancy requiring systemic treatment within the last 3 years or which would impede evaluation of treatment response.

11. Medical issue that limits oral ingestion or impairment of gastrointestinal function that is expected to significantly reduce the absorption of Compound A.

12. Clinically significant (i.e., active) cardiovascular disease: cerebral vascular accident/stroke (<6 months prior to enrollment), myocardial infarction (<6 months prior to enrollment), unstable angina, congestive heart failure (≥ New York Heart Association Classification Class II), or the presence of any condition that can increase proarrhythmic risk (e.g., hypokalemia, bradycardia, heart block) including any new, unstable, or serious cardiac arrhythmia requiring medication, or other baseline arrhythmia that might interfere with interpretation of ECGs on study (e.g., bundle branch block). Patients with QTcF>450 msec for males and >470 msec for females on screening ECG are excluded. Any patients with a bundle branch block will be excluded with QTcF>450 msec. Males who are on stable doses of concomitant medication with known prolongation of QTcF (e.g., Selective Serotonin Reuptake Inhibitor Antidepressants) are only excluded for QTcF>470 msec.

13. Patients taking strong CYP3A4/5 inhibitors (e.g., aprepitant, clarithromycin, itraconazole, ketoconazole, nefazodone, posaconazole, telithromycin, verapamil, and voriconazole) or inducers (e.g., phenytoin, rifampin, carbamazepine, St John's Wort, bosentan, modafinil, and nafcillin) are excluded from the study unless they can be transferred to other medications within ≥5 half-lives prior to dosing. Concomitant use of drugs that are strong CYP3A inhibitors or inducers on study should be avoided.

14. Patients taking concomitant medications that are metabolized solely through or are sensitive substrates of CYP3A4/5, CYP2C8, CYP2C9, CYP2B6, p-glycoprotein or breast cancer resistance protein (BCRP) transporters and have a narrow therapeutic window (e.g., repaglinide, warfarin, phenytoin, alfentanil, cyclosporine, diergotamine, ergotamine, fentanyl, pimozide, quinidine, sirolimus, efavirenz, bupropion, ketamine, methadone, propofol, tramadol, and tacrolimus) should be cautioned regarding their use and provided acceptable alternatives when possible.

15. Has an active infection requiring systemic therapy.

16. A woman of child-bearing potential (WOCBP) who has a positive pregnancy test prior to treatment.

17. Is breastfeeding or expecting to conceive or father children within the projected duration of the study, starting with the Screening visit through 120 days after the last dose of study treatment.

### Number of Subjects (Planned):

It is anticipated that approximately 50 patients will be enrolled in the study. The overall sample size for this study depends on the observed DLT profiles of Compound A. A target sample size of 26 subjects for the dose-escalation is planned, which includes four dose levels of 3 subjects each, prior to reaching the fifth planned dose which is planned to include 14 subjects to confirm the RP2D.

Of the 14 subjects at the preliminary RP2D, it is anticipated that 11 to 14 subjects will have urothelial carcinoma (however, additional subjects may be enrolled to enable a minimum of 11 efficacy evaluable subjects with urothelial carcinoma). The sample size for the first stage of the Simon 2-stage will be based on the subset of subjects with urothelial carcinoma from the dose-escalation phase that were treated at the RP2D. The total sample size from the Simon 2-stage will be 28 subjects.

Subjects who are withdrawn from treatment during the DLT period for reasons other than study drug-related AEs will be replaced.

### Treatment Groups and Duration:

### Single-Dose Run-In Period

During the Single-dose Run-in period, subjects are treated with a single dose Compound A in a fasted state at the assigned dose level prior to entering the Treatment period. For the purposes of the Single-dose Run-in period, unless otherwise indicated by or discussed with the Sponsor, the fasted state is defined as no solid food or liquids except water and medication from midnight of the night preceding the single dose to 2 hours after taking the dose. PK sampling occurs, as indicated on the Schedule of Events (SoE), to compare fed versus fasted Compound A administration.

### Treatment Period

A cycle of treatment is defined as every 3 weeks, or q3w.

Compound A, beginning at a dose of 200 mg QD is initially administered orally (PO) in a fed state (i.e., within 30 minutes of consuming a meal containing ≥6 grams of fat prior to taking Compound A daily, but should otherwise maintain a normal diet, unless modifications are required to manage an AE such as diarrhea, nausea, or vomiting). The preliminary successive dose levels of Compound A to be explored include 400 mg QD, 800 mg QD, 1200 mg QD, and 1600 mg given as 800 mg q12h given daily. Doses above 1200 mg are expected to be dosed q12h such that the total dose would be split evenly between two doses (e.g., a 1600 mg dose is given as 800 mg q12h). If feasibility issues arise (e.g., difficulty in ingesting the number of tablets) or PK indicates non-proportional increases in Compound A exposure, doses can be divided into twice daily (BID or q12h), 3 times per day (TID or q8h), or four times a day (QID or q6h). Any subject who requires a decrease in the Compound A dose below 50 mg QD will have treatment discontinued. If continuous treatment is deemed intolerable, alternate schedules (e.g., 2 weeks on/1 week off or 3 weeks on/1 week off) can be explored.

If evaluation of Compound A's clinical PK, pharmacodynamics, feasibility (e.g., exceeding the maximum number of tablets that can be ingested at one time), or safety suggests that it may be desirable to give a frequency of administration other than once a day (QD), then a new cohort of subjects can be enrolled to the highest total daily dose of Compound A evaluated to date and that is less than or equal to the MTD. In this new cohort of subjects, the same total dose given over 24 hours is administered as three times a day (TID or q8h), or four times a day (QID or q6h) regimen, depending on the available PK profile data (e.g., 1200 mg dose can be given 400 mg TID or q8h). If this division of the dose is well tolerated, then dose escalation can resume with such divided dosing in all new subjects enrolled to the study. At any time, BID dosing can be exchanged in new subjects for q12h dosing or TID with q8h dosing, or QID with q6h dosing, including in planned doses.

Subjects will not initially receive prophylactic treatment with anti-emetics. However, anti-emetics may be used to treat established Compound A-related nausea and/or vomiting prior to defining a DLT. Grade 1 or 2 diarrhea can be treated with standard dose loperamide.

Compound A-related inflammation will not be treated with systemic corticosteroids unless it proves to be dose-limiting.

Additional dose adjustment and monitoring plan is described in the protocol.

The duration of the study for each subject will include a Screening period for inclusion in the study, a Single-dose Run-in period to assess the food effect on Compound A of up to 7 days and no fewer than 2 days prior to starting the Treatment Period, courses of Compound A treatment cycles repeated every 3 weeks (i.e., 21 days), an End of Treatment 30-Day Follow-up visit, and an End of Treatment 90-Day Follow-up/End of Study visit. Subjects can continue treatment until disease progression, unacceptable toxicity, or consent withdrawal, followed by a minimum of 30-Day and 90-Day Follow-up visits after the last study drug administration. Treatment beyond disease progression using iRECIST is available for patients with urothelial carcinoma at the discretion of the Investigator.

The expected enrollment period is 29 months to the end of Stage 1 (dose-escalation) and 30 months to the end of Stage 2 (preliminary antitumor effect).

The trial cut-off date is defined as the date when all the subjects either have had 16 weeks of treatment completed or discontinued the study treatment. Subjects who continue to demonstrate clinical benefit are eligible to receive Compound A treatment until disease progression or voluntary withdrawal from the study. Study treatment terminates after 2 years of study treatment, regardless of disease progression or voluntary withdrawal from the study. Study treatment can be provided via an extension of the study, a rollover study requiring approval by responsible health authority and ethics committee, or through another mechanism at the discretion of the Sponsor.

### Statistical Considerations:

Determination of the Sample Size:

The overall sample size for this study depends on the observed DLT profiles of Compound A. A target sample size of 26 subjects for the dose-escalation and 67 subjects for dose expansion is planned.

The sample size for the first stage of the Simon 2-stage is based on the subset of urothelial carcinoma subjects from the dose-escalation phase that were treated at the selected expansion dose for the Simon 2-stage design. At least 14 patients at with urothelial carcinoma are enrolled at the selected expansion dose. The total sample size from the Simon 2-stage design is 28 subjects with urothelial carcinoma.

Specifically, there would need to be at least 1 response in the 11 to 14 initial subjects with urothelial carcinoma, and a total of 4 responses among 28 subjects to indicate further study of the drug based on this design in this population of subjects at alpha=0.05, 1-sided, excluding the null hypothesis of a response rate of 0.05 or less. The expected response rate is 0.20. The power for this design is approximately 0.80 to 0.83. Based on expected enrollment rates, the Sponsor may elect not to pause enrollment between Stage 1 and 2.

### Results

Dose cohorts comprising three (3) subjects each, in the fed state, of 200 mg, 400 mg, 800 mg, and 1200 mg (QD or once a day) of Compound A were completed without any drug-related serious adverse events (SAEs).

Interim cohort pharmacokinetics were assessed on parent (Compound A) and two active metabolites (Compound B and Compound C). Increased exposure with increase in dose observed for all three analytes (Compound A, Compound B, Compound C). PK appears greater than dose proportional on Cycle 2 Day 1 (C2D1) for all three analytes. Steady state PK was achieved for all three analytes by Day 8. Compound B metabolite ratio is increased on C2D1 in cohorts above 200 mg dosages. Accumulation of Compound B observed with repeat dosing above 200 mg. AUC (area under the curve) for Compound B is greater than Compound A, with repeated dosing for 2/3 subjects at 400 and 800 mg. Without wishing to be bound or limited by theory, elimination rate limited kinetics likely contributing to the accumulation of Compound B through on-target inhibition of CYP1A1.

The ratio of Compound B to Compound A on C2D1 was nearly identical at the 800 mg dose compared to the 400 mg dose (1.3-1.4× parent). The ratio of Compound C to Compound A was also similar at the 800 mg dose as that observed at the 400 mg dose (AUC 15-20% of parent)

Based on these results, Compound B and Compound C can be considered as “active” metabolites based on exposure and potency (in addition to Compound A). The AUC 0-24, or exposure after 24 hours, for Compound B is similar or greater than parent compound, Compound A. The IC₅₀ for Compound B is about 4 times greater than for parent compound, Compound A.

Pharmacodynamic (PD) modulation of AHR target genes were analyzed in a whole blood assay. Robust inhibition of expression of an AHR target gene, CYP1B1, was observed in all subjects in the 200 mg, 400 mg, and 800 mg cohorts.

While we have described a number of embodiments of this invention, it is apparent that our basic examples may be altered to provide other embodiments that utilize the compounds and methods of this invention. Therefore, it will be appreciated that the scope of this invention is to be defined by the application and claims rather than by the specific embodiments that have been represented by way of example.

1. A spray dried intermediate (SDI) formulation comprising compound A, 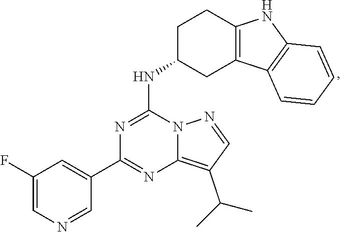 [SMILES: CC(C)c1cnn2c(N[C@@H]3CCc4[nH]c5ccccc5c4C3)nc(-c3cncc(F)c3)nc12]
or a pharmaceutically acceptable salt thereof, and a pharmaceutically acceptable polymer.

2. The SDI formulation of claim 1, comprising compound A free base.

3. The SDI formulation of claim 1, comprising compound A hemi-maleate.

4. The SDI formulation of any one of claims 1-3, wherein the pharmaceutically acceptable polymer is selected from PVP-VA, HPMC, HPMCP-55, HPMCAS-M, TPGS, HPMCAS-L, and MCC.

5. The SDI formulation of any one of claims 1-4, comprising about 25-40% wt compound A, or a pharmaceutically acceptable salt thereof.

6. The SDI formulation of any one of claims 1-5, wherein the pharmaceutically acceptable polymer is about 60-75% wt.

7. The SDI formulation of any one of claims 1-6, comprising 40:60 (wt %) compound A free base:HPMCAS-M.

8. A unit dosage form comprising the SDI formulation of any one of claims 1-7.

9. The unit dosage form of claim 8, wherein the SDI formulation is about 55-65 wt % of the unit dosage form.

10. The unit dosage form of claim 8 or 9, which is an immediate release (IR) tablet.

11. The unit dosage form of any one of claims 8-10, further comprising a filler selected from mannitol and lactose.

12. The unit dosage form of any one of claims 8-11, further comprising a disintegrant Ac-Di-Sol.

13. The unit dosage form of any one of claims 8-12, further comprising a thickening agent Cab-O-Sil.

14. The unit dosage form of any one of claims 8-13, further comprising sodium stearyl fumarate.

15. The unit dosage form of any one of claims 8-14, further comprising a binder HPC Nisso SSL SFP.

16. The unit dosage form of any one of claims 8-15, which has a full release in about 3 minutes in a sink dissolution test.

17. A method for treating cancer in a patient, comprising administering to the patient a therapeutically effect amount of the SDI formulation of any one of claims 1-7, or the unit dosage form of any one of claims 8-16.

18. The method of claim 17, wherein the cancer is selected from a hematological cancer, lymphoma, myeloma, leukemia, a neurological cancer, skin cancer, breast cancer, prostate cancer, colorectal cancer, lung cancer, head and neck cancer, gastrointestinal cancer, liver cancer, pancreatic cancer, genitourinary cancer, bone cancer, renal cancer, and vascular cancer.

19. The method of claim 17, wherein the cancer is selected from:
urothelial carcinoma, for example, bladder cancer or transitional cell carcinoma;
head and neck squamous cell carcinoma;
melanoma, for example, a uveal melanoma;
ovarian cancer, for example, a serous subtype of ovarian cancer;
renal cell carcinoma, for example, a clear cell renal cell carcinoma subtype;
cervical cancer;
gastrointestinal/stomach (GIST) cancer, for example, a stomach cancer;
non-small cell lung cancer (NSCLC), for example, advanced and/or metastatic NSCLC;
acute myeloid leukemia (AML); and
esophageal cancer.

20. The method of any one of claims 17-19, wherein the method comprises administering to the patient about 200-1600 mg (for example, about 200 mg, about 400 mg, about 600 mg, about 800 mg, about 1000 mg, about 1200 mg, or about 1600 mg) of compound A, or a pharmaceutically acceptable salt thereof, daily.

21. Use of a therapeutically effect amount of the SDI formulation of any one of claims 1-7, or the unit dosage form of any one of claims 8-16, for treating cancer in a patient.

22. The use of claim 21, wherein the cancer is selected from a hematological cancer, lymphoma, myeloma, leukemia, a neurological cancer, skin cancer, breast cancer, prostate cancer, colorectal cancer, lung cancer, head and neck cancer, gastrointestinal cancer, liver cancer, pancreatic cancer, genitourinary cancer, bone cancer, renal cancer, and vascular cancer.

23. The use of claim 21, wherein the cancer is selected from:
urothelial carcinoma, for example, bladder cancer or transitional cell carcinoma;
head and neck squamous cell carcinoma;
melanoma, for example, a uveal melanoma;
ovarian cancer, for example, a serous subtype of ovarian cancer;
renal cell carcinoma, for example, a clear cell renal cell carcinoma subtype;
cervical cancer;
gastrointestinal/stomach (GIST) cancer, for example, a stomach cancer;
non-small cell lung cancer (NSCLC), for example, advanced and/or metastatic NSCLC;
acute myeloid leukemia (AML); and
esophageal cancer.

24. The use of any one of claims 21-23, wherein the SDI formulation or the unit dosage form comprises about 200-1600 mg (for example, about 200 mg, about 400 mg, about 600 mg, about 800 mg, about 1000 mg, about 1200 mg, or about 1600 mg) of compound A, or a pharmaceutically acceptable salt thereof, and is administered daily.
